SEQUENCE LISTING (110 sequences)

SEQ ID NO: 1 - protein, 145 aa, Arachis hypogaea
  REGION 1..145: note = A5Z1R0
MAKSTILVALLALVLVAHASAMRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

SEQ ID NO: 2 - protein, 124 aa, synthetic construct
  REGION 1..124: note = Ara h 6 WT
MRRERGRQGDSSSCERQVDRVNLKPCEQHIMQRIMGEQEQYDSYDIRSTRSSDQQQRCCDELNEMENTQRCMCEALQQIMENQCDRLQDRQMVQQFKRELMNLPQQCNFRAPQRCDLDVSGGRC

SEQ ID NO: 3 - protein, 123 aa, synthetic construct
  REGION 1..123: note = Arah6_D6
RDEDGDAGASRSCERQVDRVNLDPCESHIMQRIMGAAEQYKSYAIRSTRSSDQQQRCCDELNEMENTQRCMCEALQQIMENQCDRLQNDAMVQQFKRELMNLPQQCPFRAPQDCKLRVSGGRC

SEQ ID NO: 4 - protein, 123 aa, synthetic construct
  REGION 1..123: note = Arah6_D7
RDEDGDSGSSDSCERQVDRVNLDPCESHIMQRIMGTSEQYESYQIRSTRSSDQQQRCCDELNEMENTQRCMCEALQQIMENQCDRLQNDSMVQQFKRELMNLPQQCEFRAPQHCKLTVSGGRC

SEQ ID NO: 5 - protein, 123 aa, synthetic construct
  REGION 1..123: note = Arah6_D8
RRERGRQGDSNSCRRRVDRVFLDPCESHIMQRIMGEQEQYDSYDIRSTRSSDQQQDCYDFLNSMENTQRCMCEALQLIMATNCDRLQRRQMVQQFIDMLMNLPQQCNFRAPQACMLDVSGGRC

SEQ ID NO: 6 - protein, 123 aa, synthetic construct
  REGION 1..123: note = Arah6_D9
RDEDGRSGDSSSCERQVDRVNLKPCEQHIMQRIMGEQEQYDSYAIRSTRSSDQQQDCYDFLNSMENTQRCMCEALQLIMANKCDRLQRRQMVQQFIDMLMNLPQQCNFRAPQACMLDVSGGRC

SEQ ID NO: 7 - protein, 123 aa, synthetic construct
  REGION 1..123: note = Arah6_D10
RDEDGDSGKSSSCERQVDRVNLKPCEQHIMQRIMGEQEQYGSYASRSTRSSDQQQRCCDELNEMENTQRCMCEALQQIMENQCDRLQGDQMVQQFKRELMNLPQQCDFEAPDDCKLRVSGGRC

SEQ ID NO: 8 - protein, 123 aa, synthetic construct
  REGION 1..123: note = Arah6_D11
SSEDGRSGDSSSCESQVDDVNLKPCEQHIMQRIMGSQESYDSYDGSSTRSSDQQQRCCDELNEMENTQRCMCEALQQIMENQCDSLQDDQMVQQFKDELMNLPQQCNFSAPIGCDLDVSGGDC

SEQ ID NO: 9 - protein, 123 aa, synthetic construct
  REGION 1..123: note = Arah6_D12
RDEDGRAGDSSSCERQVDRVNLDPCESHIMQRIAGAAEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 10 - protein, 123 aa, synthetic construct
  REGION 1..123: note = Arah6_D13
RDEDGRAGDSSSCERQVDRVNLDPCESHIMQRIAGAAEQYDSYARRSTRSSDQQQDCYDFLNSMENTQRCMCEALQLIMATNCDRLQRDQMVQQFIDMLMNLPQQCDFEAPQACMLRVSGGRC

SEQ ID NO: 11 - protein, 123 aa, synthetic construct
  REGION 1..123: note = Arah6_D71
RDEDGRAGDSSSCEDQVQRVNLDPCESHIMQRIAGAAEQDDSYARRSTRSSDQQQRCCDELNEMENTQRCMCRALQQIMRNQCDRLQGDQMVQQFKLELMNLPQQCDFEAPQDCKLRVSGGRC

SEQ ID NO: 12 - protein, 123 aa, synthetic construct
  REGION 1..123: note = Arah6_D72
RDEDGRAGDSSSCEDQVLRVNLDPCESHIMQRIAGAAEQDDSYARRSTRSSDQQQRCCDELNEMENTQRCMCRALQQIMRNQCDRLQGDQMVQQFKLELMNLPQQCDFEAPQDCKLRVSGGRC

SEQ ID NO: 13 - protein, 123 aa, synthetic construct
  REGION 1..123: note = Arah6_D73
RDEDGRAGDSSSCEDQVQRVNLDPCESHIMQRIAGAAEQDDSYDRRSTRSSDQQQRCCDELNEMENTQRCMCRALQQIMRNQCDRLQGDQMVQQFKLELMNLPQQCDFEAPQDCKLDVSGGRC

SEQ ID NO: 14 - protein, 123 aa, synthetic construct
  REGION 1..123: note = Arah6_D74
RDEDGRAGDSSSCEDQVQRVNLKPCEQHIMQRIMGEQEQDDSYARRSTRSSDQQQRCCDELNEMENTQRCMCRALQQIMRNQCDRLQGDQMVQQFKLELMNLPQQCDFEAPQDCKLRVSGGRC

SEQ ID NO: 15 - protein, 123 aa, synthetic construct
  REGION 1..123: note = Arah6_D75
RDEDGRAGDSSSCEDQVQRVNLDPCESHIMQRIAGAAEQDDSYDRRSTRSSDQQQRCCDELNEMENTQRCMCEALQQIMENQCDRLQGDQMVQQFKLELMNLPQQCDFEAPQDCKLDVSGGRC

SEQ ID NO: 16 - protein, 123 aa, synthetic construct
  REGION 1..123: note = Arah6_D76
RDEDGRAGDSSSCEDQVQRVNLKPCEQHIMQRIMGEQEQDDSYDRRSTRSSDQQQRCCDELNEMENTQRCMCEALQQIMENQCDRLQGDQMVQQFKLELMNLPQQCDFEAPQDCKLDVSGGRC

SEQ ID NO: 17 - protein, 123 aa, synthetic construct
  REGION 1..123: note = Arah6_D77
RDEDGRAGDSSSCEDQVQRVNLKPCEQHIMQRIMGEQEQDDSYARRSTRSSDQQQRCCDELNEMENTQRCMCEALQQIMENQCDRLQGDQMVQQFKLELMNLPQQCDFEAPQDCKLRVSGGRC

SEQ ID NO: 18 - protein, 123 aa, synthetic construct
  REGION 1..123: note = Arah6_D78
RDEDGRAGDSSSCEDQVLRVNLKPCEQHIMQRIMGEQEQDDSYARRSTRSSDQQQRCCDELNEMENTQRCMCRALQQIMRNQCDRLQGDQMVQQFKLELMNLPQQCDFEAPQDCKLRVSGGRC

SEQ ID NO: 19 - protein, 123 aa, synthetic construct
  REGION 1..123: note = Arah6_D79
RDEDGRAGDSSSCEDQVLRVNLKPCEQHIMQRIMGEQEQDDSYARRSTRSSDQQQRCCDELNEMENTQRCMCEALQQIMENQCDRLQGDQMVQQFKLELMNLPQQCDFEAPQDCKLRVSGGRC

SEQ ID NO: 20 - protein, 123 aa, synthetic construct
  REGION 1..123: note = Arah6_D80
RDEDGRAGDSSSCEDQVLRVNLKPCEQHIMQRIMGEQEQDDSYDRRSTRSSDQQQRCCDELNEMENTQRCMCEALQQIMENQCDRLQGDQMVQQFKLELMNLPQQCDFEAPQDCKLDVSGGRC

SEQ ID NO: 21 - protein, 123 aa, synthetic construct
  REGION 1..123: note = Arah6_D81
RDEDGRAGDSSSCEDQVDRVNLKPCEQHIMQRIMGEQEQDDSYARRSTRSSDQQQRCCDELNEMENTQRCMCEALQQIMENQCDRLQGDQMVQQFKLELMNLPQQCDFEAPQDCKLRVSGGRC

SEQ ID NO: 22 - protein, 124 aa, synthetic construct
  VARIANT 2: note = Any amino acid
  VARIANT 3: note = Any amino acid
  VARIANT 5: note = Any amino acid
  VARIANT 7: note = Any amino acid
  VARIANT 8: note = Any amino acid
  VARIANT 10: note = Any amino acid
  VARIANT 12: note = Any amino acid
  VARIANT 15: note = Any amino acid
  VARIANT 16: note = Any amino acid
  VARIANT 17: note = Any amino acid
  VARIANT 19: note = Any amino acid
  VARIANT 20: note = Any amino acid
  VARIANT 22: note = Any amino acid
  VARIANT 24: note = Any amino acid
  VARIANT 28: note = Any amino acid
  VARIANT 35: note = Any amino acid
  VARIANT 37: note = Any amino acid
  VARIANT 38: note = Any amino acid
  VARIANT 40: note = Any amino acid
  VARIANT 41: note = Any amino acid
  VARIANT 42: note = Any amino acid
  VARIANT 45: note = Any amino acid
  VARIANT 46: note = Any amino acid
  VARIANT 47: note = Any amino acid
  VARIANT 57: note = Any amino acid
  VARIANT 59: note = Any amino acid
  VARIANT 61: note = Any amino acid
  VARIANT 64: note = Any amino acid
  VARIANT 74: note = Any amino acid
  VARIANT 78: note = Any amino acid
  VARIANT 81: note = Any amino acid
  VARIANT 82: note = Any amino acid
  VARIANT 83: note = Any amino acid
  VARIANT 86: note = Any amino acid
  VARIANT 89: note = Any amino acid
  VARIANT 90: note = Any amino acid
  VARIANT 91: note = Any amino acid
  VARIANT 97: note = Any amino acid
  VARIANT 98: note = Any amino acid
  VARIANT 99: note = Any amino acid
  VARIANT 108: note = Any amino acid
  VARIANT 110: note = Any amino acid
  VARIANT 113: note = Any amino acid
  VARIANT 114: note = Any amino acid
  VARIANT 116: note = Any amino acid
  VARIANT 118: note = Any amino acid
  VARIANT 123: note = Any amino acid
MXXEXGXXGXSXSCXXXVXXVXLXPCEXHIMQRIXGXXEXXXSYXXXSTRSSDQQQXCXDXLNXMENTQRCMCXALQXIMXXXCDXLQXXXMVQQFXXXLMNLPQQCXFXAPXXCXLXVSGGXC

SEQ ID NO: 23 - protein, 124 aa, synthetic construct; identical to SEQ ID NO: 22
  VARIANT 2: note = R or S
  VARIANT 3: note = R, D or S
  VARIANT 5: note = R or D
  VARIANT 7: note = R or D
  VARIANT 8: note = Q, A or S
  VARIANT 10: note = D, A, S, or K
  VARIANT 12: note = S, R, D, or N
  VARIANT 15: note = E or R
  VARIANT 16: note = R, S or D
  VARIANT 17: note = Q or R
  VARIANT 19: note = D, Q or L
  VARIANT 20: note = R or D
  VARIANT 22: note = N or F
  VARIANT 24: note = K or D
  VARIANT 28: note = Q or S
  VARIANT 35: note = M or A
  VARIANT 37: note = E, A, T, or S
  VARIANT 38: note = Q, A or S
  VARIANT 40: note = Q or S
  VARIANT 41: note = Y or D
  VARIANT 42: note = D, K, E, or G
  VARIANT 45: note = D, A or Q
  VARIANT 46: note = I, S, G or R
  VARIANT 47: note = R or S
  VARIANT 57: note = R or D
  VARIANT 59: note = C or Y
  VARIANT 61: note = E or F
  VARIANT 64: note = E or S
  VARIANT 74: note = E or R
  VARIANT 78: note = Q or L
  VARIANT 81: note = E, A or R
  VARIANT 82: note = N or T
  VARIANT 83: note = Q, N, or K
  VARIANT 86: note = R or S
  VARIANT 89: note = D, N, R, or G
  VARIANT 90: note = R or D
  VARIANT 91: note = Q, A, or S
  VARIANT 97: note = K or I
  VARIANT 98: note = R, D or L
  VARIANT 99: note = E or M
  VARIANT 108: note = N, P, E or D
  VARIANT 110: note = R, E or S
  VARIANT 113: note = Q, D or I
  VARIANT 114: note = R, D, H, A or G
  VARIANT 116: note = D, K or M
  VARIANT 118: note = D, R or T
  VARIANT 123: note = R or D

SEQ ID NO: 24 - protein, 123 aa, synthetic construct
  REGION 1..123: note = Arah6_D82
RDEDGRAGDSSSCEDQVRRVNLDPCESHIMQRIAGAAEQDDSYDRRSTRSSDQQQRCCDELNEMENTQRCMCEALQQIMENQCDRLQGDQMVQQFKDELMNLPQQCDFEAPQDCKLDVSGGRC

SEQ ID NO: 25 - protein, 123 aa, synthetic construct
  REGION 1..123: note = Arah6_D83
RDEDGRAGDSSSCEDQVRRVNLKPCEQHIMQRIMGEQEQDDSYDRRSTRSSDQQQRCCDELNEMENTQRCMCEALQQIMENQCDRLQGDQMVQQFKDELMNLPQQCDFEAPQDCKLDVSGGRC

SEQ ID NO: 26 - protein, 123 aa, synthetic construct
  REGION 1..123: note = Arah6_D84
RDEDGRAGDSSSCEDQVQRVNLDPCESHIMQRIAGAAEQYDSYDRRSTRSSDQQQRCCDELNEMENTQRCMCEALQQIMENQCDRLQGDQMVQQFKDELMNLPQQCDFEAPQDCKLDVSGGRC

SEQ ID NO: 27 - protein, 123 aa, synthetic construct
  REGION 1..123: note = Arah6_D85
RDEDGRAGDSSSCEDQVQRVNLKPCEQHIMQRIMGEQEQYDSYDRRSTRSSDQQQRCCDELNEMENTQRCMCEALQQIMENQCDRLQGDQMVQQFKDELMNLPQQCDFEAPQDCKLDVSGGRC

SEQ ID NO: 28 - protein, 123 aa, synthetic construct
  REGION 1..123: note = Arah6_D86
RDEDGRAGDSSSCEDQVRRVNLDPCESHIMQRIAGAAEQDDSYDRRSTRSSDQQQRCCDELNEMENTQRCMCEALQQIMENQCDRLQGDQMVQQFKLELMNLPQQCDFEAPQDCKLRVSGGRC

SEQ ID NO: 29 - protein, 123 aa, synthetic construct
  REGION 1..123: note = Arah6_D87
RDEDGRAGDSSSCEDQVRRVNLKPCEQHIMQRIMGEQEQDDSYDRRSTRSSDQQQRCCDELNEMENTQRCMCEALQQIMENQCDRLQGDQMVQQFKLELMNLPQQCDFEAPQDCKLRVSGGRC

SEQ ID NO: 30 - protein, 123 aa, synthetic construct
  REGION 1..123: note = Arah6_D88
RDEDGRAGDSSSCEDQVQRVNLDPCESHIMQRIAGAAEQYDSYDRRSTRSSDQQQRCCDELNEMENTQRCMCEALQQIMENQCDRLQGDQMVQQFKLELMNLPQQCDFEAPQDCKLRVSGGRC

SEQ ID NO: 31 - protein, 123 aa, synthetic construct
  REGION 1..123: note = Arah6_D89
RDEDGRAGDSSSCEDQVQRVNLKPCEQHIMQRIMGEQEQYDSYDRRSTRSSDQQQRCCDELNEMENTQRCMCEALQQIMENQCDRLQGDQMVQQFKLELMNLPQQCDFEAPQDCKLRVSGGRC

SEQ ID NO: 32 - protein, 123 aa, synthetic construct
  REGION 1..123: note = Arah6_D90
RDEDGRAGDSSSCEDQVQRVNLDPCESHIMQRIAGAAEQDDSYDRRSTRSSDQQQRCCDELNEMENTQRCMCEALQQIMENQCDRLQGDQMVQQFKLELMNLPQQCDFEAPQDCKLRVSGGRC

SEQ ID NO: 33 - protein, 123 aa, synthetic construct
  REGION 1..123: note = Arah6_D91
RDEDGRAGDSSSCEDQVQRVNLKPCEQHIMQRIMGEQEQDDSYDRRSTRSSDQQQRCCDELNEMENTQRCMCEALQQIMENQCDRLQGDQMVQQFKLELMNLPQQCDFEAPQDCKLRVSGGRC

SEQ ID NO: 34 - protein, 123 aa, synthetic construct
  REGION 1..123: note = Arah6_D92
RDEDGRAGDSSSCEDQVRRVNLDPCESHIMQRIAGAAEQYDSYDRRSTRSSDQQQRCCDELNEMENTQRCMCEALQQIMENQCDRLQGDQMVQQFKDELMNLPQQCDFEAPQDCKLDVSGGRC

SEQ ID NO: 35 - protein, 123 aa, synthetic construct
  REGION 1..123: note = Arah6_D93
RDEDGRAGDSSSCEDQVRRVNLKPCEQHIMQRIMGEQEQYDSYDRRSTRSSDQQQRCCDELNEMENTQRCMCEALQQIMENQCDRLQGDQMVQQFKDELMNLPQQCDFEAPQDCKLDVSGGRC

SEQ ID NO: 36 - protein, 123 aa, synthetic construct
  REGION 1..123: note = Arah6_D94
RDEDGRAGDSSSCEDQVRRVNLDPCESHIMQRIAGAAEQYDSYDRRSTRSSDQQQRCCDELNEMENTQRCMCEALQQIMENQCDRLQGDQMVQQFKDELMNLPQQCDFEAPQDCKLRVSGGRC

SEQ ID NO: 37 - protein, 123 aa, synthetic construct
  REGION 1..123: note = Arah6_D95
RDEDGRAGDSSSCEDQVRRVNLKPCEQHIMQRIMGEQEQYDSYDRRSTRSSDQQQRCCDELNEMENTQRCMCEALQQIMENQCDRLQGDQMVQQFKDELMNLPQQCDFEAPQDCKLRVSGGRC

SEQ ID NO: 38 - protein, 123 aa, synthetic construct
  REGION 1..123: note = Arah6_D96
RDEDGRAGDSSSCEDQVQRVNLDPCESHIMQRIAGAAEQYDSYDRRSTRSSDQQQRCCDELNEMENTQRCMCEALQQIMENQCDRLQGDQMVQQFKDELMNLPQQCDFEAPQDCKLRVSGGRC

SEQ ID NO: 39 - protein, 123 aa, synthetic construct
  REGION 1..123: note = Arah6_D97
RDEDGRAGDSSSCEDQVQRVNLKPCEQHIMQRIMGEQEQYDSYDRRSTRSSDQQQRCCDELNEMENTQRCMCEALQQIMENQCDRLQGDQMVQQFKDELMNLPQQCDFEAPQDCKLRVSGGRC

SEQ ID NO: 40 - protein, 123 aa, synthetic construct
  REGION 1..123: note = Arah6_D98
RDEDGRAGDSSSCEDQVRRVNLDPCESHIMQRIAGAAEQYDSYDRRSTRSSDQQQRCCDELNEMENTQRCMCEALQQIMENQCDRLQGDQMVQQFKLELMNLPQQCDFEAPQDCKLRVSGGRC

SEQ ID NO: 41 - protein, 123 aa, synthetic construct
  REGION 1..123: note = Arah6_D99
RDEDGRAGDSSSCEDQVRRVNLKPCEQHIMQRIMGEQEQYDSYDRRSTRSSDQQQRCCDELNEMENTQRCMCEALQQIMENQCDRLQGDQMVQQFKLELMNLPQQCDFEAPQDCKLRVSGGRC

SEQ ID NO: 42 - protein, 123 aa, synthetic construct
  REGION 1..123: note = Arah6_D100
RDEDGRAGDSSSCEDQVQRVNLDPCESHIMQRIAGAAEQYDSYDRRSTRSSDQQQRCCDELNEMENTQRCMCEALQQIMENQCDRLQGDQMVQQFKLELMNLPQQCDFEAPQDCKLDVSGGRC

SEQ ID NO: 43 - protein, 123 aa, synthetic construct
  REGION 1..123: note = Arah6_D101
RDEDGRAGDSSSCEDQVQRVNLKPCEQHIMQRIMGEQEQYDSYDRRSTRSSDQQQRCCDELNEMENTQRCMCEALQQIMENQCDRLQGDQMVQQFKLELMNLPQQCDFEAPQDCKLDVSGGRC

SEQ ID NO: 44 - protein, 123 aa, synthetic construct; identical to SEQ ID NO: 15
  REGION 1..123: note = Arah6_D113

SEQ ID NO: 45 - protein, 123 aa, synthetic construct; identical to SEQ ID NO: 16
  REGION 1..123: note = Arah6_D114

SEQ ID NO: 46 - protein, 123 aa, synthetic construct
  REGION 1..123: note = Arah6_D119
RDEDGRAGDSSSCERQVRRVNLKPCEQHIMQRIAGAAEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 47 - protein, 123 aa, synthetic construct
  REGION 1..123: note = Arah6_D120
RDEDGRAGDSSSCERQVRRVNLDPCESHIMQRIMGEQEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 48 - protein, 123 aa, synthetic construct
  REGION 1..123: note = Arah6_D121
RDEDGRAGDSSSCERQVRRVNLKPCEQHIMQRIMGEQEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 49 - protein, 123 aa, synthetic construct
  REGION 1..123: note = Arah6_D122
RDEDGRAGDSSSCERQVRRVNLKPCEQHIMQRIAGAAEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 50 - protein, 123 aa, synthetic construct
  REGION 1..123: note = Arah6_D123
RDEDGRAGDSSSCERQVRRVNLDPCESHIMQRIMGEQEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 51 - protein, 123 aa, synthetic construct
  REGION 1..123: note = Arah6_D124
RDEDGRAGDSSSCERQVRRVNLKPCEQHIMQRIMGEQEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 52 - protein, 123 aa, synthetic construct
  REGION 1..123: note = Arah6_D125
RDEDGRAGDSSSCERQVDRVNLKPCEQHIMQRIAGAAEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 53 - protein, 123 aa, synthetic construct
  REGION 1..123: note = Arah6_D126
RDEDGRAGDSSSCERQVDRVNLDPCESHIMQRIMGEQEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 54 - protein, 123 aa, synthetic construct
  REGION 1..123: note = Arah6_D127
RDEDGRAGDSSSCERQVDRVNLKPCEQHIMQRIMGEQEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 55 - protein, 123 aa, synthetic construct
  REGION 1..123: note = Arah6_D128
RDEDGRAGDSSSCERQVDRVNLKPCEQHIMQRIAGAAEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 56 - protein, 123 aa, synthetic construct
  REGION 1..123: note = Arah6_D129
RDEDGRAGDSSSCERQVDRVNLDPCESHIMQRIMGEQEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 57 - protein, 123 aa, synthetic construct
  REGION 1..123: note = Arah6_D130
RDEDGRAGDSSSCERQVDRVNLKPCEQHIMQRIMGEQEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 58 - protein, 123 aa, synthetic construct
  REGION 1..123: note = Arah6_D131
RDEDGRAGDSSSCERQVRRVNLDPCESHIMQRIAGAAEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 59 - protein, 123 aa, synthetic construct
  REGION 1..123: note = Arah6_D132
RDEDGRAGDSSSCERQVDRVNLDPCESHIMQRIAGAAEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 60 - protein, 123 aa, synthetic construct
  REGION 1..123: note = Arah6_D133
RDEDGRAGDSSSCERQVRRVNLDPCESHIMQRIAGAAEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 61 - protein, 123 aa, synthetic construct
  REGION 1..123: note = Arah6_D134
RDEDGRAGDSSSCERQVDRVNLKPCESHIMQRIAGAAEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 62 - protein, 123 aa, synthetic construct
  REGION 1..123: note = Arah6_D135
RDEDGRAGDSSSCERQVDRVNLDPCEQHIMQRIAGAAEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 63 - protein, 123 aa, synthetic construct
  REGION 1..123: note = Arah6_D136
RDEDGRAGDSSSCERQVDRVNLDPCESHIMQRIMGAAEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 64 - protein, 123 aa, synthetic construct
  REGION 1..123: note = Arah6_D137
RDEDGRAGDSSSCERQVDRVNLDPCESHIMQRIAGEAEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 65 - protein, 123 aa, synthetic construct
  REGION 1..123: note = Arah6_D138
RDEDGRAGDSSSCERQVDRVNLDPCESHIMQRIAGAQEQYDSYARRSTRSSDQQQRCCDELNEMENTQRCMCEALQQIMENQCDRLQGDQMVQQFKRELMNLPQQCDFEAPQDCKLRVSGGRC

SEQ ID NO: 66 - protein, 123 aa, synthetic construct
  REGION 1..123: note = Arah6_D144
RDEDGRAGDSSSCERQVRRVNLKPCEQHIMQRIAGAAEQYDSYARRSTRSSDQQQRCCDELNEMENTQRCMCEALQQIMENQCDDLQGDQMVQQFKDELMNLPQQCDFEAPQDCKLRVSGGRC

SEQ ID NO: 67 - protein, 123 aa, synthetic construct
  REGION 1..123: note = Arah6_D145
RDEDGRAGDSSSCERQVRRVNLKPCEQHIMQKIAGAAEQYDSYARRSTRSSDQQQRCCDELNEMENTQRCMCEALQQIMENQCDRLQGDQMVQQFKDELMNLPQKCDFEAPQDCKLRVSGGRC

SEQ ID NO: 68 - protein, 123 aa, synthetic construct
  REGION 1..123: note = Arah6_D146
RDEDGRAGDSSSCERQVRRVNLKPCEQHIMQKIAGAAEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 69 - protein, 123 aa, synthetic construct
  REGION 1..123: note = Arah6_D147
RDEDGRAGDSSSCERQVRRVNLKPCEQHIMQQIAGAAEQYDSYARRSTRSSDQQQRCCDELNEMENTQRCMCEALQQIMENQCDRLQGDQMVQQFKDELMNLPQKCDFEAPQDCKLRVSGGRC

SEQ ID NO: 70 - protein, 123 aa, synthetic construct
  REGION 1..123: note = Arah6_D148
RDEDGRAGDSSSCERQVRRVNLKPCEQHIMQQIAGAAEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 71 - protein, 123 aa, synthetic construct
  REGION 1..123: note = Arah6_D149
RDEDGRAGDSSSCERQVRRVNLKPCEQHIMQKIAGAAEQYDSYARRSTRSSDQQQRCCDELNEMENTQRCMCEALQQIMENQCDRLQGDQMVQQFKDELMNLPQHCDFEAPQDCKLRVSGGRC

SEQ ID NO: 72 - protein, 123 aa, synthetic construct
  REGION 1..123: note = Arah6_D150
RDEDGRAGDSSSCERQVRRVNLKPCEQHIMQKIAGAAEQYDSYARRSTRSSDQQQRCCDELNEMENTQRCMCEALQQIMENQCDDLQGDQMVQQFKDELMNLPQHCDFEAPQDCKLRVSGGRC

SEQ ID NO: 73 - protein, 123 aa, synthetic construct
  REGION 1..123: note = Arah6_D151
RDEDGRAGDSSSCERQVRRVNLKPCEQHIMQQIAGAAEQYDSYARRSTRSSDQQQRCCDELNEMENTQRCMCEALQQIMENQCDRLQGDQMVQQFKDELMNLPQHCDFEAPQDCKLRVSGGRC

SEQ ID NO: 74 - protein, 123 aa, synthetic construct
  REGION 1..123: note = Arah6_D152
RDEDGRAGDSSSCERQVRRVNLKPCEQHIMQQIAGAAEQYDSYARRSTRSSDQQQRCCDELNEMENTQRCMCEALQQIMENQCDDLQGDQMVQQFKDELMNLPQHCDFEAPQDCKLRVSGGRC

SEQ ID NO: 75 - protein, 123 aa, synthetic construct
  REGION 1..123: note = Arah6_D153
RDEDGRAGDSSSCERQVRRVNLKPCEQHIMQRIAGAAEQYDSYARRSTRSSDQQQRCCDELNEMENTQRCMCEALQQIMENQCDRLQGDQMVQQFKDELMNLPQKCDFEAPQDCKLRVSGGRC

SEQ ID NO: 76 - protein, 123 aa, synthetic construct
  REGION 1..123: note = Arah6_D154
RDEDGRAGDSSSCERQVRRVNLKPCEQHIMQRIAGAAEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 77 - protein, 123 aa, synthetic construct
  REGION 1..123: note = Arah6_D155
RDEDGRAGDSSSCERQVRRVNLKPCEQHIMQRIMGAAEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 78 - protein, 123 aa, synthetic construct
  REGION 1..123: note = Arah6_D156
RDEDGRAGDSSSCERQVRRVNLKPCEQHIMQRIAGEAEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 79 - protein, 123 aa, synthetic construct
  REGION 1..123: note = Arah6_D157
RDEDGRAGDSSSCERQVRRVNLKPCEQHIMQRIAGAQEQYDSYARRSTRSSDQQQRCCDELNEMENTQRCMCEALQQIMENQCDRLQGDQMVQQFKDELMNLPQQCDFEAPQDCKLRVSGGRC

SEQ ID NO: 80 - protein, 123 aa, synthetic construct
  REGION 1..123: note = Arah6_D158
RDEDGRAGDSSSCERQVRRVNLKPCEQHIMQRIMGEQEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 81 - protein, 123 aa, synthetic construct
  REGION 1..123: note = Arah6_D159
RDEDGRAGDSSSCERQVRRVNLKPCEQHIMQRIAGEQEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 82 - protein, 123 aa, synthetic construct
  REGION 1..123: note = Arah6_D160
RDEDGRAGDSSSCERQVRRVNLKPCEQHIMQRIMGAQEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 83 - protein, 123 aa, synthetic construct
  REGION 1..123: note = Arah6_D161
RDEDGRAGDSSSCERQVRRVNLKPCEQHIMQRIMGEAEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 84 - protein, 123 aa, synthetic construct
  REGION 1..123: note = Arah6_D162
RDEDGRAGDSSSCERQVRRVNLKPCEQHIMQRIMGAAEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 85 - protein, 123 aa, synthetic construct
  REGION 1..123: note = Arah6_D163
RDEDGRAGDSSSCERQVRRVNLKPCEQHIMQRIAGEAEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 86 - protein, 123 aa, synthetic construct
  REGION 1..123: note = Arah6_D164
RDEDGRAGDSSSCERQVRRVNLKPCEQHIMQRIAGAQEQYDSYARRSTRSSDQQQRCCDELNEMENTQRCMCEALQQIMENQCDDLQGDQMVQQFKDELMNLPQKCDFEAPQDCKLRVSGGRC

SEQ ID NO: 87 - protein, 123 aa, synthetic construct
  REGION 1..123: note = Arah6_D165
RDEDGRAGDSSSCERQVRRVNLKPCEQHIMQRIMGEQEQYDSYARRSTRSSDQQQRCCDELNEMENTQRCMCEALQQIMENQCDDLQGRQMVQQFKDELMNLPQKCDFEAPQDCKLRVSGGRC

SEQ ID NO: 88 - protein, 123 aa, synthetic construct
  REGION 1..123: note = Arah6_D166
RDEDGRAGDSSSCERQVRRVNLKPCEQHIMQRIAGEQEQYDSYARRSTRSSDQQQRCCDELNEMENTQRCMCEALQQIMENQCDDLQGRQMVQQFKDELMNLPQKCDFEAPQDCKLRVSGGRC

SEQ ID NO: 89 - protein, 123 aa, synthetic construct
  REGION 1..123: note = Arah6_D167
RDEDGRAGDSSSCERQVRRVNLKPCEQHIMQRIMGAQEQYDSYARRSTRSSDQQQRCCDELNEMENTQRCMCEALQQIMENQCDDLQGRQMVQQFKDELMNLPQKCDFEAPQDCKLRVSGGRC

SEQ ID NO: 90 - protein, 123 aa, synthetic construct
  REGION 1..123: note = Arah6_D168
RDEDGRAGDSSSCERQVRRVNLKPCEQHIMQRIMGEAEQYDSYARRSTRSSDQQQRCCDELNEMENTQRCMCEALQQIMENQCDDLQGRQMVQQFKDELMNLPQKCDFEAPQDCKLRVSGGRC

SEQ ID NO: 91 - protein, 123 aa, synthetic construct
  REGION 1..123: note = Arah6_D169
RDEDGRAGDSSSCERQVRRVNLKPCEQHIMQRIMGAAEQYDSYARRSTRSSDQQQRCCDELNEMENTQRCMCEALQQIMENQCDDLQGRQMVQQFKDELMNLPQKCDFEAPQDCKLRVSGGRC

SEQ ID NO: 92 - protein, 123 aa, synthetic construct
  REGION 1..123: note = Arah6_D170
RDEDGRAGDSSSCERQVRRVNLKPCEQHIMQRIAGEAEQYDSYARRSTRSSDQQQRCCDELNEMENTQRCMCEALQQIMENQCDDLQGRQMVQQFKDELMNLPQKCDFEAPQDCKLRVSGGRC

SEQ ID NO: 93 - protein, 123 aa, synthetic construct
  REGION 1..123: note = Arah6_D171
RDEDGRAGDSSSCERQVRRVNLKPCEQHIMQRIAGAQEQYDSYARRSTRSSDQQQRCCDELNEMENTQRCMCEALQQIMENQCDDLQGRQMVQQFKDELMNLPQKCDFEAPQDCKLRVSGGRC

SEQ ID NO: 94 - protein, 123 aa, synthetic construct
  REGION 1..123: note = Arah6_D172
RDEDGRAGDSSSCERQVRRVNLKPCEQHIMQRIMGEQEQYDSYARRSTRSSDQQQRCCDELNEMENTQRCMCEALQQIMENQCDDLQGDQMVQQFKDELMNLPQKCNFEAPQDCKLRVSGGRC

SEQ ID NO: 95 - protein, 123 aa, synthetic construct
  REGION 1..123: note = Arah6_D173
RDEDGRAGDSSSCERQVRRVNLKPCEQHIMQRIAGEQEQYDSYARRSTRSSDQQQRCCDELNEMENTQRCMCEALQQIMENQCDDLQGDQMVQQFKDELMNLPQKCNFEAPQDCKLRVSGGRC

SEQ ID NO: 96 - protein, 123 aa, synthetic construct
  REGION 1..123: note = Arah6_D174
RDEDGRAGDSSSCERQVRRVNLKPCEQHIMQRIMGAQEQYDSYARRSTRSSDQQQRCCDELNEMENTQRCMCEALQQIMENQCDDLQGDQMVQQFKDELMNLPQKCNFEAPQDCKLRVSGGRC

SEQ ID NO: 97 - protein, 123 aa, synthetic construct
  REGION 1..123: note = Arah6_D175
RDEDGRAGDSSSCERQVRRVNLKPCEQHIMQRIMGEAEQYDSYARRSTRSSDQQQRCCDELNEMENTQRCMCEALQQIMENQCDDLQGDQMVQQFKDELMNLPQKCNFEAPQDCKLRVSGGRC

SEQ ID NO: 98 - protein, 123 aa, synthetic construct
  REGION 1..123: note = Arah6_D176
RDEDGRAGDSSSCERQVRRVNLKPCEQHIMQRIMGAAEQYDSYARRSTRSSDQQQRCCDELNEMENTQRCMCEALQQIMENQCDDLQGDQMVQQFKDELMNLPQKCNFEAPQDCKLRVSGGRC

SEQ ID NO: 99 - protein, 123 aa, synthetic construct
  REGION 1..123: note = Arah6_D177
RDEDGRAGDSSSCERQVRRVNLKPCEQHIMQRIAGEAEQYDSYARRSTRSSDQQQRCCDELNEMENTQRCMCEALQQIMENQCDDLQGDQMVQQFKDELMNLPQKCNFEAPQDCKLRVSGGRC

SEQ ID NO: 100 - protein, 123 aa, synthetic construct
  REGION 1..123: note = Arah6_D178
RDEDGRAGDSSSCERQVRRVNLKPCEQHIMQRIAGAQEQYDSYARRSTRSSDQQQRCCDELNEMENTQRCMCEALQQIMENQCDDLQGDQMVQQFKDELMNLPQKCNFEAPQDCKLRVSGGRC

SEQ ID NO: 101 - protein, 123 aa, synthetic construct
  REGION 1..123: note = Arah6_D179
RDEDGRAGDSSSCERQVRRVNLKPCEQHIMQRIMGEQEQYDSYARRSTRSSDQQQRCCDELNEMENTQRCMCEALQQIMENQCDDLQGRQMVQQFKDELMNLPQKCNFEAPQDCKLRVSGGRC

SEQ ID NO: 102 - protein, 123 aa, synthetic construct
  REGION 1..123: note = Arah6_D180
RDEDGRAGDSSSCERQVRRVNLKPCEQHIMQRIAGEQEQYDSYARRSTRSSDQQQRCCDELNEMENTQRCMCEALQQIMENQCDDLQGRQMVQQFKDELMNLPQKCNFEAPQDCKLRVSGGRC

SEQ ID NO: 103 - protein, 123 aa, synthetic construct
  REGION 1..123: note = Arah6_D181
RDEDGRAGDSSSCERQVRRVNLKPCEQHIMQRIMGAQEQYDSYARRSTRSSDQQQRCCDELNEMENTQRCMCEALQQIMENQCDDLQGRQMVQQFKDELMNLPQKCNFEAPQDCKLRVSGGRC

SEQ ID NO: 104 - protein, 123 aa, synthetic construct
  REGION 1..123: note = Arah6_D182
RDEDGRAGDSSSCERQVRRVNLKPCEQHIMQRIMGEAEQYDSYARRSTRSSDQQQRCCDELNEMENTQRCMCEALQQIMENQCDDLQGRQMVQQFKDELMNLPQKCNFEAPQDCKLRVSGGRC

SEQ ID NO: 105 - protein, 123 aa, synthetic construct
  REGION 1..123: note = Arah6_D183
RDEDGRAGDSSSCERQVRRVNLKPCEQHIMQRIMGAAEQYDSYARRSTRSSDQQQRCCDELNEMENTQRCMCEALQQIMENQCDDLQGRQMVQQFKDELMNLPQKCNFEAPQDCKLRVSGGRC

SEQ ID NO: 106 - protein, 123 aa, synthetic construct
  REGION 1..123: note = Arah6_D184
RDEDGRAGDSSSCERQVRRVNLKPCEQHIMQRIAGEAEQYDSYARRSTRSSDQQQRCCDELNEMENTQRCMCEALQQIMENQCDDLQGRQMVQQFKDELMNLPQKCNFEAPQDCKLRVSGGRC

SEQ ID NO: 107 - protein, 123 aa, synthetic construct
  REGION 1..123: note = Arah6_D185
RDEDGRAGDSSSCERQVRRVNLKPCEQHIMQRIAGAQEQYDSYARRSTRSSDQQQRCCDELNEMENTQRCMCEALQQIMENQCDDLQGRQMVQQFKDELMNLPQKCNFEAPQDCKLRVSGGRC

SEQ ID NO: 108 - protein, 123 aa, synthetic construct
  REGION 1..123: note = Arah6_D186
RDEDGRAGDSSSCERQVRRVNLKPCEQHIMQRIAGAAEQYDSYARRSTRSSDQQQRCCDELNEMENTQRCMCEALQQIMENQCDDLQGRQMVQQFKDELMNLPQQCDFEAPQRCKLRVSGGRC

SEQ ID NO: 109 - protein, 124 aa, synthetic construct
  VARIANT 2: note = Any amino acid
  VARIANT 3: note = Any amino acid
  VARIANT 5: note = Any amino acid
  VARIANT 7: note = Any amino acid
  VARIANT 8: note = Any amino acid
  VARIANT 10: note = Any amino acid
  VARIANT 12: note = Any amino acid
  VARIANT 15: note = Any amino acid
  VARIANT 16: note = Any amino acid
  VARIANT 17: note = Any amino acid
  VARIANT 19: note = Any amino acid
  VARIANT 20: note = Any amino acid
  VARIANT 22: note = Any amino acid
  VARIANT 24: note = Any amino acid
  VARIANT 28: note = Any amino acid
  VARIANT 33: note = Any amino acid
  VARIANT 35: note = Any amino acid
  VARIANT 37: note = Any amino acid
  VARIANT 38: note = Any amino acid
  VARIANT 40: note = Any amino acid
  VARIANT 41: note = Any amino acid
  VARIANT 42: note = Any amino acid
  VARIANT 45: note = Any amino acid
  VARIANT 46: note = Any amino acid
  VARIANT 47: note = Any amino acid
  VARIANT 57: note = Any amino acid
  VARIANT 59: note = Any amino acid
  VARIANT 61: note = Any amino acid
  VARIANT 64: note = Any amino acid
  VARIANT 74: note = Any amino acid
  VARIANT 78: note = Any amino acid
  VARIANT 81: note = Any amino acid
  VARIANT 82: note = Any amino acid
  VARIANT 83: note = Any amino acid
  VARIANT 86: note = Any amino acid
  VARIANT 89: note = Any amino acid
  VARIANT 90: note = Any amino acid
  VARIANT 91: note = Any amino acid
  VARIANT 97: note = Any amino acid
  VARIANT 98: note = Any amino acid
  VARIANT 99: note = Any amino acid
  VARIANT 106: note = Any amino acid
  VARIANT 108: note = Any amino acid
  VARIANT 110: note = Any amino acid
  VARIANT 113: note = Any amino acid
  VARIANT 114: note = Any amino acid
  VARIANT 116: note = Any amino acid
  VARIANT 118: note = Any amino acid
  VARIANT 123: note = Any amino acid
  VARIANT 63: note = Any amino acid
  VARIANT 109: note = Any amino acid
MXXEXGXXGXSXSCXXXVXXVXLXPCEXHIMQXIXGXXEXXXSYXXXSTRSSDQQQXCXDXLXXMENTQRCMCXALQXIMXXXCDXLQXXXMVQQFXXXLMNLPQXCXXXAPXXCXLXVSGGXC

SEQ ID NO: 110 - protein, 124 aa, synthetic construct
  VARIANT 2: note = R or S
  VARIANT 3: note = R, D or S
  VARIANT 5: note = R or D
  VARIANT 7: note = R or D
  VARIANT 8: note = Q, A or S
  VARIANT 10: note = D, A, S, or K
  VARIANT 12: note = S, R, D, or N
  VARIANT 15: note = E or R
  VARIANT 16: note = R, S or D
  VARIANT 17: note = Q or R
  VARIANT 19: note = D, Q, L or R
  VARIANT 20: note = R or D
  VARIANT 22: note = N or F
  VARIANT 24: note = K or D
  VARIANT 28: note = Q or S
  VARIANT 33: note = R, Q or K
  VARIANT 35: note = M or A
  VARIANT 37: note = E, A, T, or S
  VARIANT 38: note = Q, A or S
  VARIANT 40: note = Q or S
  VARIANT 41: note = Y or D
  VARIANT 42: note = D, K, E, or G
  VARIANT 45: note = D, A or Q
  VARIANT 46: note = I, S, G or R
  VARIANT 47: note = R or S
  VARIANT 57: note = R or D
  VARIANT 59: note = C or Y
  VARIANT 61: note = E or F
  VARIANT 64: note = E or S
  VARIANT 74: note = E or R
  VARIANT 78: note = Q or L
  VARIANT 81: note = E, A or R
  VARIANT 82: note = N or T
  VARIANT 83: note = Q, N, or K
  VARIANT 86: note = R, D or S
  VARIANT 89: note = D, N, R, or G
  VARIANT 90: note = R or D
  VARIANT 91: note = Q, A, or S
  VARIANT 97: note = K or I
  VARIANT 98: note = R, D or L
  VARIANT 99: note = E or M
  VARIANT 106: note = Q, K, or H
  VARIANT 108: note = N, P, E or D
  VARIANT 110: note = R, E or S
  VARIANT 113: note = Q, D or I
  VARIANT 114: note = R, D, H, A or G
  VARIANT 116: note = D, K or M
  VARIANT 118: note = D, R or T
  VARIANT 123: note = R or D
MXXEXGXXGXSXSCXXXVXXVXLXPCEXHIMQXIXGXXEXXXSYXXXSTRSSDQQQXCXDXLNXMENTQRCMCXALQXIMXXXCDXLQXXXMVQQFXXXLMNLPQXCXFXAPXXCXLXVSGGXC

(19) United States
(12) Patent Application Publication
(10) Pub. No.: US 2026/0356896 A1
(43) Pub. Date: Nov. 12, 2026

(54) COMPOSITIONS AND METHODS FOR INDUCING DESENSITIZATION TO PEANUTS

(71) Applicant: Ukko Inc., San Francisco, CA (US)

(72) Inventors: Yanay OFRAN, Tel Aviv (IL); Moshe BEN DAVID, Rehovot (IL); Orly MARCU GARBER, Modi'in (IL); Almog BREGMAN COHEN, Rehovot (IL); Zohar BIRON SOREK, Rehovot (IL); Almog BITTON, Rishon LeZiyon (IL)

(73) Assignee: Ukko Inc., San Francisco, CA (US)

(21) Appl. No.: 19/123,862

(22) PCT Filed: Nov. 1, 2023
(86) PCT No.: PCT/US2023/078294

§ 371 Date: Apr. 24, 2025

Related U.S. Application Data
(60) Provisional application No. 63/421,685, filed on Nov. 2, 2022.

(51) Int. Cl. C07K 14/415; A23L 25/00; A61K 39/35; A61P 37/08; C12N 15/82

(52) U.S. Cl. CPC C07K 14/415; A23L 25/20; A61K 39/35; A61P 37/08; C12N 15/8243

(57) ABSTRACT

In one embodiment, the present disclosure provides hypoallergenic peanut allergens Ara h 6 variants wherein at least one epitope recognized by anti-Ara h 6 antibodies has been modified, thereby having reduced or abolished antibody binding to the peanut allergen. These peanut allergen variants are hypoallergenic but maintain their immunogenicity and may be used in methods of treating subjects allergic to peanuts and reducing the severity of their allergy.

### SEQUENCE LISTING

The instant application contains a Sequence Listing which has been submitted electronically in XML file format and is hereby incorporated by reference in its entirety. Said XML copy, created on Oct. 29, 2023, is named P-621156-PC_SL.xml and is 219,755 bytes in size.

### FIELD OF INVENTION

The disclosure relates in general to recombinant hypoallergenic peanut allergen Ara h 6, methods of producing same, and uses thereof.

### BACKGROUND

One of the most severe food allergies known today is peanut allergy, where allergic individuals respond to exposure to peanuts, even at low concentrations, with symptoms ranging from mild, local effects, to severe, life-threatening effects. Peanuts are the leading cause for food induced anaphylactic shock in the United States (Finkelman, (2010) Current Opinion in Immunology, 22(6):783-788) and some form of allergic reaction to peanuts is reported in around 1% of the US population (Sicherer S H, et al., (2010). J Allergy Clin Immunol. 125(6):1322-6).

So far, 17 peanut proteins have been proposed to be involved in IgE mediated allergic reaction (Palladino, C., & Breiteneder, H. (2018). Molecular immunology, 100:58-70). Of these proteins, the seed storage proteins Ara h 1, Ara h 2, Ara h 3 and Ara h 6 are considered major allergens, those whose recognition by an IgE antibody mediated response is correlated with more severe symptoms (Palladino, et al., 2018; ibid) (Bernard, et al., (2007) J Agric Food Chem. 55(23):9663-9).

Ara h 6, a member of the 2S albumin family, is a major allergen from peanut (Arachis hypogaea). Ara h 6 has 145 amino acids including a 21-amino acid signal peptide, is dominated by five alpha-helices and contains five intramolecular disulfide bonds (uniport A5Z1R0; Q647G9).

Both Ara h 2 and Ara h 6 belong to the conglutin type of 2S-albumins. Conformational models of Ara h 2 and Ara h 6 are virtually superposable, indicating near similar tertiary structures of the two proteins. Currently they are considered the most potent peanut allergens (Kulis, Mike, et al. (2012) Clinical & Experimental Allergy; 42.2: 326-336). Ara h 2 and Ara h 6 are the most frequently recognized major peanut allergens in children (Flinterman, A. E., et al. (2007) Clinical & Experimental Allergy 37.8: 1221-1228; and van Erp, Francine C., et al. (2017) Journal of Allergy and Clinical Immunology; 139.1: 358-360). The individual reactivity to the major peanut allergens remained stable over time (Flinterman A E, et al., (2007) Clin Exp Allergy; 37(8) 1221-1228).

Because of their structural similarity, anti-Ara h 6 antibodies often cross-react with Ara h 2 and vice versa (Koppelman, S. J., et al. (2005) Clinical & Experimental Allergy; 35.4 (2005): 490-497). Nevertheless, the proteins are not identical and there are putative antibody epitopes in each of them that do not exist on the other. It has been reported that there are allergic patients who are sensitized to only one peanut 2S albumin protein (Asarnoj A, Glaumann S, Elfstrom L, et al. (2012) Int Arch Allergy Immunol.; 159(2):209-212). Such a report suggests that IgE antibodies against the specific epitopes in Ara h 6 that are not present in Ara h 2 may suffice to trigger clinical reaction at least in some patients.

There remains an unmet need for hypoallergenic peanut proteins and methods of use thereof for standardized immunotherapeutic treatment, in subjects allergic to peanut allergens.

### SUMMARY

Described herein are several epitope mapping approaches for identifying epitopes on Ara h 6 and protein engineering approaches for designing hypoallergenic Ara h 6 allergen variants that maintain biophysical and functional characteristics. In one aspect, disclosed herein are recombinant Ara h 6 variant polypeptides lacking at least one epitope recognized by an anti-Ara h 6 antibody, thereby reducing or abolishing IgE antibody binding to the variant polypeptides. In some embodiments, an epitope comprises a linear epitope. In some embodiments, an epitope comprises a conformational epitope. In another aspect, these variant polypeptides may be used in methods of inducing desensitization to peanuts and/or immunomodulation in human patients allergic to peanuts.

In one aspect, provided herein is a recombinant Ara h 6 variant polypeptide.

In some embodiments, the recombinant Ara h 6 variant polypeptide comprises an amino acid sequence comprising one or more amino acid substitutions, deletions, insertions, or any combination thereof, that are located at one or more of positions 3, 5, 8, 19, 45, 46, 86, 89, 90, 98, 106, 108, 110, 114, 116, or 118 of SEQ ID NO: 109, as compared with the amino acid residues at those same positions in SEQ ID NO: 2.

In some embodiments, the recombinant Ara h 6 variant polypeptide comprises one or more amino acid substitutions, deletions, insertions, or any combination thereof, that are located at one or more of positions 2, 3, 5, 7, 8, 10, 12, 16, 19, 22, 24, 33, 37, 38, 40, 41, 42, 45, 46, 47, 74, 78, 81, 82, 83, 86, 89, 90, 97, 98, 99, 106, 108, 110, 113, 114, 116, or 118 of SEQ ID NO: 109, as compared with the amino acid residues at those same positions in SEQ ID NO: 2.

In some embodiments, the recombinant Ara h 6 variant polypeptide comprises one or more amino acid substitutions, deletions, insertions, or any combination thereof, that are located at one or more of positions 2, 3, 5, 7, 8, 10, 12, 16, 19, 22, 24, 33, 37, 38, 40, 41, 42, 45, 46, 47, 63, 74, 78, 81, 82, 83, 86, 89, 90, 97, 98, 99, 106, 108, 109, 110, 113, 114, 116, or 118 of SEQ ID NO: 109, as compared with the amino acid residues at those same positions in SEQ ID NO: 2.

In some embodiments, the recombinant Ara h 6 variant comprises one or more of the following substitutions:
(a) S at position 2;
(b) D, or S at position 3;
(c) D at position 5;
(d) D at position 7;
(e) A, or S at position 8;
(f) A, S, or K at position 10;
(g) R, D, or N at position 12;
(h) S, or D at position 16;
(i) Q, L, or R at position 19;
(j) F at position 22;
(k) D at position 24;
(l) Q, or K at position 33;
(m) A, T, or S at position 37;
(n) A, or S at position 38;
(o) S at position 40;
(p) D at position 41;
(q) K, E, or G at position 42;
(r) A, or Q at position 45;
(s) S, G, or R at position 46;
(t) S at position 47;
(u) R at position 74;
(v) L at position 78;
(w) A, or R at position 81;
(x) T at position 82;
(y) N, or K at position 83;
(z) D, or S at position 86;
(aa) N, R, or G at position 89;
(bb) D at position 90;
(cc) I at position 97;
(dd) D, or L at position 98;
(ee) M at position 99;
(ff) K, or H at position 106;
(gg) P, E, or D at position 108;
(hh) E, or S at position 110;
(ii) D, or I at position 113;
(jj) D, H, A, or G at position 114;
(kk) K, or M at position 116; or
(ll) R, or T at position 118.

In some embodiments, the variant comprises one or more amino acid substitutions, deletions, insertions, or any combination thereof, that are located at positions 15, 17, 20, 28, 35, 57, 59, 61, 64, 91, or 123 of SEQ ID NO: 109, as compared with the amino acid residues at those same positions in SEQ ID NO: 2.

In some embodiments, the variant comprises amino acid substitutions, deletions, insertions, or any combination thereof, that are located at positions 3, 5, 8, 19, 45, 46, 89, 98, 110, 114, 116, and 118 of SEQ ID NO: 109, as compared with the amino acid residues at those same positions in SEQ ID NO: 2.

In some embodiments, the substitutions comprise one or more of: D, or S at position 3; D at position 5; A, or S at position 8; Q, L, or R at position 19; A, or Q at position 45; S, G, or R at position 46; N, R, or G at position 89; D, or L at position 98; E, or S at position 110; D, H, A, or G at position 114; K, or M at position 116; or R, or T at position 118.

In some embodiments, the variant further comprises amino acid substitutions, deletions, insertions, or any combination thereof, at one or more of positions 2, 7, 10, 12, 15, 16, 17, 20, 22, 24, 28, 33, 35, 37, 38, 40, 41, 42, 47, 57, 59, 61, 63, 64, 74, 78, 81, 82, 83, 86, 90, 91, 97, 99, 106, 108, 109, 113, or 123 of SEQ ID NO: 109, as compared with the amino acid residues at those same positions in SEQ ID NO: 2.

In some embodiments, the substitutions comprise one or more of:
(a) S at position 2;
(b) D at position 7;
(c) A, S, or K at position 10;
(d) R, D, or N at position 12;
(e) R at position 15;
(f) S, or D at position 16;
(g) R at position 17;
(h) D at position 20;
(i) F at position 22;
(j) D at position 24;
(k) S at position 28;
(l) Q, or K at position 33;
(m) A at position 35;
(n) A, T, or S at position 37;
(o) A, or S at position 38;
(p) S at position 40;
(q) D at position 41;
(r) K, E, or G at position 42;
(s) S at position 47;
(t) D at position 57;
(u) Y at position 59;
(v) F at position 61;
(w) S at position 64;
(x) R at position 74;
(y) L at position 78;
(z) A, or R at position 81;
(aa) T at position 82;
(bb) N, or K at position 83;
(cc) S, or D at position 86
(dd) D at position 90
(ee) A, or S at position 91;
(ff) I at position 97;
(gg) M at position 99;
(hh) K, or H at position 106;
(ii) P, E, or D at position 108;
(jj) D, or I at position 113; and
(kk) D at position 123.

In some embodiments, the variant comprises one or more amino acid substitutions, deletions, insertions, or any combination thereof that are located within at least a single epitope recognized by an anti-Ara h 6 antibody.

In some embodiments, the variant comprises one or more amino acid substitutions, deletions, insertions, or any combination thereof that are located within at least 2 epitopes, e.g., within 2, 3, 4, 5, 6, 7, 8, 9, 10 or more epitopes, recognized by an anti-Ara h 6 antibody.

In some embodiments, the variant comprises an amino acid sequence that is at least 80% identical to the amino acid sequence as set forth in any of SEQ ID NOs: 3-21 or SEQ ID NOs: 24-108.

In some embodiments, the variant comprises the amino acid sequence as set forth in any of SEQ ID NOs: 3-21 or SEQ ID NOs: 24-108.

In some embodiments, the recombinant Ara h 6 variant comprises between 2-30 substitutions, deletions, insertions, or any combination thereof.

In some embodiments, “SEQ ID NO:110” and “SEQ ID NO:109” are used herein interchangeably.

Also provided herein are a nucleotide or modified nucleotide sequence encoding any one of the above recombinant Ara h 6 variants, an expression vector comprising the nucleotide or modified nucleotide sequence, as well as a cell comprising the expression vector. There is also provided a method of using the expression vector to produce any one of the above recombinant Ara h 6 variants disclosed herein.

In some embodiments, the nucleotide or modified nucleotide sequence is DNA or mRNA. In some embodiments, the mRNA is encapsulated within a nanoparticle. In some embodiments, the mRNA comprises LNP-formulated mRNA.

In some embodiments, the cell comprising the expression vector is a prokaryotic cell or a eukaryotic cell. In some embodiments, the eukaryotic cell is a yeast cell, a fungi cell, a plant cell, or a mammalian cell.

In another aspect, the present disclosure also provides a composition comprising the recombinant Ara h 6 variant polypeptide and/or the isolated nucleotide or modified nucleotide sequence encoding the recombinant Ara h 6 variant disclosed herein. In some embodiments, the composition is a pharmaceutical composition comprising an acceptable carrier or excipient.

In another aspect, the present disclosure also provides a method of inducing desensitization to peanuts and/or immunomodulation of a response to peanuts in a subject allergic to peanuts, the method comprises administering to the subject a composition comprising the recombinant Ara h 6 variant disclosed herein, thereby inducing desensitization to peanuts and/or immunomodulation of a response to peanuts in the subject.

In another aspect, the present disclosure also provides a method of inducing desensitization to peanuts and/or immunomodulation of a response to peanuts in a subject allergic to peanuts, the method comprises administering to the subject a composition comprising isolated nucleotide or modified nucleotide sequence encoding the recombinant Ara h 6 variant disclosed herein, thereby inducing desensitization to peanuts and/or immunomodulation of a response to peanuts in the subject.

In another aspect, the present disclosure also provides a genetically modified peanut plant expressing the recombinant Ara h 6 variant polypeptide disclosed herein, or a combination thereof. In some embodiments, the expression is from a heterologous nucleic acid. In some embodiments, the expression of an endogenous wild-type Ara h 6 allergen is reduced compared with a non-genetically modified peanut.

In another aspect, the present disclosure also provides a processed food product comprising the Ara h 6 variant disclosed herein. In some embodiments, the processed food product comprises a reduced amount of endogenous wild-type peanut Ara h 6 allergen. In some embodiments, the processed food product comprises a peanut harvested from the genetically modified plant disclosed herein.

In some embodiments, the composition is for use in inducing desensitization to peanuts and/or in immunomodulation in a subject in need thereof. In some embodiments, the composition as disclosed herein is for use in the preparation of a medicament for inducing desensitization to peanuts and/or in immunomodulation in a subject in need thereof.

### BRIEF DESCRIPTION OF THE DRAWINGS

The subject matter regarded the hypoallergenic polypeptide variants described herein having reduced allergenicity while maintaining immunogenicity, and methods of making the same is particularly pointed out and distinctly claimed in the concluding portion of the specification. The engineered Ara h 6 polypeptide variants and methods of making the same, however, both as to organization and method of operation, together with objects, features, and advantages thereof, may best be understood by reference to the following detailed description when read with the accompanying drawings in which:

FIGS. 1A and 1B. Linear epitope mapping and de-epitoping reveals mutations that abolish binding to Ara h 6 epitopes. FIG. 1A: Linear epitope mapping of mAb IgG8 (mAb isolated from a peanut allergic patient) reveals IgE binding to peptide of Ara h 6. Black box highlights an Ara h 6 mapped epitope L2 (a peptide derived from positions 61-79 of SEQ ID NO: 1). FIG. 1B: Linear de-epitoping of patient mAb IgG8 Ara h 6 epitopes. Black box highlights the same peptide as in FIG. 1A. The gray box highlights a spot where a point mutation dramatically reduced binding to L2.

FIG. 2. Two main linear epitope regions of IgE binding were mapped by peptide array using plasma samples from 80 peanut allergic patients. Ara h 6 peptide binding by patient plasma or sera at the population level is being calculated for each peptide by the relative deviation from slide median intensity (Z-like score). The distribution of all scores from all slides is plotted and shown as a box plot, where the x-axis corresponds to all overlapping peptides and the y-axis shows the distribution of Z-like scores. Black and gray lines signify 2 and 3 standard deviations, respectively, from the slide median intensities.

FIG. 3A-3B. Expression and purification of Ara h 6 wild type (WT) and de-epitoped Ara h 6 variant D12 (3A) and Ara h 6 WT and de-epitoped Ara h 6 variants D154, D158, D160 and D179 (3B). Ara h 6 WT and variants expressed in E. coli and purified by immobilized metal affinity chromatography (IMAC) and size-exclusion chromatography (SEC), were incubated in Laemmli buffer for 5 min at 90 °C under reducing conditions. Samples were run on TG-SDS PAGE using 4-20% polyacrylamide gel and stained with Coomassie. Natural Ara h 6 is shown in the first lane for comparison (3B).

FIG. 4A-4F. Size exclusion chromatogram trace (SEC)-HPLC analysis of the purified constructs Ara h 6 WT (4A), D12 (4B), D154 (4C), D158 (4D), D160 (4E), and D179 (4F) variants show that both purified constructs are stable and run as the monomeric forms under the standard conditions tested. Equal volumes of both samples were injected at ambient temperature into an XBridge Protein BEH SEC 200 Å column on a UHPLC Arc System (2.5 μm, Waters cat #186009176). Each sample was run at a flow rate of 0.3 mL/min in a mobile phase of 100 mM sodium phosphate buffer, pH 7.4, 200 mM NaCl at 30 °C.

FIGS. 5A and 5B. Modified Ara h 6 variant maintains high thermal stability. Circular dichroism (CD) analysis of recombinant Ara h 6 WT and D12 variant is presented. CD spectroscopy was performed using a Chirascan v.4.7.0.194 CD spectrometer on the two intact constructs. Far-UV CD spectra at wavelengths 190-260 nm, with a step-size of 0.5 nm, were recorded at 15 temperatures, from 20 °C to 90 °C, in smooth ramp mode at a ramp-rate of 1 °C per min. Data are shown for Ara h 6 WT (5A) and D12 variant (5B) at 25 °C and incremental temperatures at ranges of 20-90 °C exhibiting similar secondary structure composition of the variant relative to the WT, suggesting no significant deviation from the natural fold.

FIGS. 6A-6G. Modified Ara h 6 variants exhibit dramatically reduced activation potential of basophils compared to Ara h 6 WT and Ara h 6 natural (nAra h 6). For allergenic potential evaluation of different Ara h 6 variants, rat basophilic leukemia (RBL) SX-38 cell degranulation assay was conducted. RBL SX-38 cells were sensitized with allergic patients' plasma or serum for 18 hours. Cells were then treated with Ara h 6 WT, Ara h 6 natural, keyhole limpet hemocyanin (KLH), as negative control, Ara h 6 variants (D12, D75, D76, D77, D154, D158, D160, or D179) for 1 hour at concentrations ranging from 2 ug/ml to 0.02 ng/ml. Degranulation was measured using β-Hexosaminidase activity assay. Results of Ara h 6 variants D12, D75, D76, and D77 are shown for six patient plasma, denoted R560 (6A), R568 (6B), CL592 (6C), A601 (6D), A604 (6E), and A608 (6F). Results of Ara h 6 variants D154, D158, D160, or D179 are shown as an average of 11 peanut allergic patients' plasma (6G).

FIG. 7. Ara h 6 WT and de-epitoped Ara h 6 D12 are expressed, folded, and secreted from mammalian cells. 20 ml of Expi293F cells were transfected with 20 micrograms of plasmid encoding for the either Ara h 6 WT, or Ara h 6 D12, both constructs expressing downstream of a human osteonectin leader sequence and containing a C terminal 6× his tag, using Expifectamine293 transfection reagent according to the manufacturer's instructions. The cells were left to express the protein for 5 days in Expi293 medium at 37 degrees, 8% CO2. The secreted protein was purified from the expression medium using Ni-NTA superflow beads, washed and eluted with the addition 0f 350 mM imidazole. The eluted fractions were analyzed by SDS PAGE, either reduced by β-mercaptoethanol (β-ME) or non-reduced.

FIGS. 8A-8F. Ara h 6 D12 variant shows reduced binding to anti-Ara h 6 mAbs, 4 IgGs and 2 IgEs. Indirect ELISA titration with increasing concentrations of the anti Ara h 6 mAb was used to test binding to WT recombinant Ara h 6 (SEQ ID NO: 2) or modified Ara h 6 D12 variant (SEQ ID NO: 9), KLH was used as a negative control. The data presented demonstrates that modified Ara h 6 D12 variant shows dramatically reduced binding to 2 anti-Ara h 6 IgEs: E15C2 (8A) and 7B6 (8B) and 4 anti-Ara h 6 IgGs: IgG5 (8C), IgG8 (8D), IgG18 (8E) and IgG24 (8F).

### DETAILED DESCRIPTION

In the following detailed description, numerous specific details are set forth in order to provide a thorough understanding of the recombinant Ara h 6 allergen variants disclosed and uses thereof. However, it will be understood by those skilled in the art that the recombinant Ara h 6 allergen variants described and uses thereof, may be practiced without these specific details. In other instances, well-known methods, procedures, and components have not been described in detail so as not to obscure the present recombinant Ara h 6 variants described and uses thereof.

In some embodiments, recombinant Ara h 6 variants were mutated based on data collected during the epitope mapping process. Mutation sites were selected based on the likelihood of a mutation, alone or in combination with additional mutations, to alter or destroy one or more epitopes recognized by anti-Ara h 6 antibodies. The allergenicity of Ara h 6 variants was assessed by rat basophil leukemia (RBL) with peanut-allergic patient samples. The desired immunogenicity, i.e., the ability of the engineered Ara h 6 to trigger a response of the immune system without triggering mast cells/basophils mediated allergic reaction, is measured by T cell activation assays.

A skilled artisan would appreciate that the term “epitope” may be used interchangeably with the term “antigenic determinant” having all the same meanings and qualities and may encompass a site on an antigen to which an immunoglobulin or antibody (or antigen binding fragment thereof) specifically binds. Epitopes can be formed both from sequence-contiguous amino acids and from sequence-noncontiguous amino acids that form a spatially contiguous patch by the tertiary folding of a protein. Epitopes formed from sequence-contiguous amino acids may also be linear epitopes, which are epitopes that bind the immunoglobulin also as peptides, outside the context of the folded protein. Such linear epitopes are typically capable of binding their cognate immunoglobulins after exposure to denaturing solvents, whereas epitopes formed by tertiary folding (conformational epitopes) typically lose binding upon treatment with denaturing solvents. In some embodiments, the epitope is as small as possible while still maintaining immunogenicity. Immunogenicity is indicated by the ability to elicit an immune response, as described herein, for example, by the ability to bind an MHC class II molecule and to induce a T cell response, e.g., by measuring T cell cytokine production.

As used herein, “de-epitoped Ara h 6 allergen” refers to a modified Ara h 6 allergen that has reduced or abolished binding with anti-Ara h 6 antibodies (as compared to antibody binding to the wild-type Ara h 6) due to mutation(s) at one or more epitopes recognized by the anti-Ara h 6 antibodies. In one embodiment, the de-epitoped Ara h 6 allergen has reduced allergenicity as compared to its wild-type counterpart.

As used herein, an “epitope” refers to the part of a macromolecule (e.g., Ara h 6 allergen) that is bound by an antibody or an antigen-binding fragment thereof. Within a protein sequence, there are linear and continuous epitopes (“linear epitopes”), which are composed of sequence-contiguous amino acids, or discontinuous epitopes, which are composed of amino acids that are discontinuous in sequence but are brought together to create a contiguous patch in the folded protein (“conformational epitopes”).

As used herein, an “allergen” refers to a substance, protein, or non-protein, capable of inducing allergy or specific hypersensitivity.

As used herein, “allergenicity” or “allergenic” refers to the ability of an antigen or allergen to induce an abnormal immune response, which is an overreaction and different from a normal immune response in that it does not result in a protective/prophylaxis effect but instead causes physiological function disorder or tissue damage.

As used herein, “hypoallergenic” refers to a substance having little or reduced likelihood of causing an allergic response.

In some embodiments, the present disclosure provides peanut allergen (e.g., Ara h 6) variants that were mutated to diminish or abolish binding of one or more epitopes by anti-peanut allergen antibodies. In one embodiment, the mutation does not affect, or affects only minimally, the biophysical and/or functional characteristics of the peanut allergen. The mutation in one aspect may be substitution, deletion, or insertion, or any combination thereof. A deletion, for example, may comprise the removal of a single amino acid that is crucial for antibody binding, or of a whole mapped epitope region.

Six general classes of amino acid side chains have been categorized and include: Class I (Cys); Class II (Ser, Thr, Ala, Gly); Class III (Asn, Asp, Gln, Glu); Class IV (His, Arg, Lys); Class V (Ile, Leu, Val, Met); and Class VI (Phe, Tyr, Trp). Conservative amino acid substitution refers to substitution of an amino acid in one class by an amino acid of the same class. For example, substitution of an Asp for another class III residue such as Asn, Gln, or Glu, is a conservative substitution. Non-conservative amino acid substitution refers to substitution of an amino acid in one class with an amino acid from another class: for example, substitution of an Ala, a class II residue, with a class III residue such as Asp, Asn, Glu, or Gln. Methods of substitution mutations at the nucleotide or amino acid sequence level are well-known in the art.

The term “modifying”, or “modification”, as used herein, refers to changing one or more amino acids in an antigen, epitope, allergen or allergen region. The change can be produced by adding, substituting, or deleting an amino acid at one or more positions. The change can be produced using known techniques, such as PCR mutagenesis. For example, in some embodiments, an antigen, epitope, allergen or allergen region identified using the methods provided herein can be modified, to thereby modify (e.g., decrease or eliminate) the binding affinity of the antibody or antigen-binding portion thereof to the peanut allergen.

### Ara h 6 Variants

In one embodiment, the present disclosure provides a recombinant Ara h 6 variant polypeptide comprising an amino acid sequence that is at least 75%, such as at least 77%, at least 80% or higher, identical to the sequence set forth in SEQ ID NO: 2, wherein the Ara h 6 variant comprises one or more amino acid substitutions, deletions, insertions, or any combination thereof, that are located within a single epitope recognized by an anti-Ara h 6 antibody. In another embodiment, the Ara h 6 variant comprises one or more amino acid substitutions, deletions, insertions, or any combination thereof, that are located within at least two epitopes recognized by anti-Ara h 6 antibodies.

A skilled artisan would appreciate that percent identity (% identity) provides a number that describes how similar the query sequence is to the target sequence (i.e., how many amino acids in each sequence are identical). The higher the percent identity is, the more significant the match.

When used in relation to polypeptide (or protein) sequences, the term “identity” refers to the degree of identity between two or more polypeptide (or protein) sequences or fragments thereof. Typically, the degree of similarity between two or more polypeptide (or protein) sequences refers to the degree of similarity of the composition, order, or arrangement of two or more amino acids of the two or more polypeptides (or proteins).

In some embodiments, the variant Ara h 6 polypeptides comprises an amino acid sequence that is at least 70%, at least 75%, at least 77%, at least 80%, at least 85%, at least 90%, at least 95%, or at least 99% identical to a polypeptide or a portion thereof disclosed herein, as determined using BlastP software of the National Center of Biotechnology Information (NCBI) using default parameters.

In some embodiments, the Ara h 6 variants may encompass deletion, insertion, or amino acid substitution mutations. In one embodiment, the variant polypeptide comprises conservative substitutions, or deletions, insertions, or substitutions that do not radically alter the three-dimensional structure of the polypeptide of interest described herein. In some embodiments, the deletion, insertion, or substitution does not alter the function of the polypeptide of interest disclosed herein. In some embodiments, the deletion, insertion, or substitution does not alter the potential to induce the immune system's response and generate desensitization to the peanut allergen.

In some embodiments, the recombinant Ara h 6 variant polypeptide comprises one or more substitution, deletion, insertion, or any combination thereof. In some embodiments, the recombinant Ara h 6 variant polypeptide comprises between 2-30 substitutions, deletions, insertions, or any combination thereof, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, or any range therebetween.

In one embodiment, the recombinant Ara h 6 variant polypeptide comprising the amino acid sequence set forth in SEQ ID NO: 22, wherein the variant comprises substitutions, deletions, insertions, or any combination thereof, at one or more of positions of SEQ ID NO: 22, as compared with the amino acid residues at those same positions 3, 5, 8, 16, 19, 24, 28, 41, 45, 46, 74, 81, 89, 90, 98, 108, 110, 114, 116, or 118 in SEQ ID NO: 2. In one embodiment, the substitution mutation is D or S at position 3. In one embodiment, the substitution mutation is D at position 5. In one embodiment, the substitution mutation is A or S at position 8. In one embodiment, the substitution mutation is S or D at position 16. In one embodiment, the substitution mutation is Q, L or R at position 19. In one embodiment, the substitution mutation is D at position 24. In one embodiment, the substitution mutation is S at position 28. In one embodiment, the substitution mutation is D at position 41. In one embodiment, the substitution mutation is A or Q at position 45. In one embodiment, the substitution mutation is S, G or R at position 46. In one embodiment, the substitution mutation is R at position 74. In one embodiment, the substitution mutation is A or R at position 81. In one embodiment, the substitution mutation is N, R, or G at position 89. In one embodiment, the substitution mutation is D at position 90. In one embodiment, the substitution mutation is D or L at position 98. In one embodiment, the substitution mutation is P, E or D at position 108. In one embodiment, the substitution mutation is E or S at position 110. In one embodiment, the substitution mutation is D, H, A or G at position 114. In one embodiment, the substitution mutation is K or M at position 116. In one embodiment, the substitution mutation is R or T at position 118.

In some embodiments of the above recombinant Ara h 6 variants, the Ara h 6 variants comprise at least 2, 3, 4, 5, 6, 7, 8, 9, 10, 11, 12, 13, 14, 15, 16, 17, 18, or 19 substitution mutations at least one position selected from positions 3, 5, 8, 16, 19, 24, 28, 41, 45, 46, 74, 81, 89, 90, 98, 108, 110, 114, 116, or 118 of SEQ ID NO: 22, as compared with the amino acid residues at those same positions in SEQ ID NO: 2.

In some embodiments of the above recombinant Ara h 6 variants, the Ara h 6 variants further comprise at least one additional substitution deletion, insertion or any combination thereof at one or more of positions 2, 7, 10, 12, 15, 17, 20, 22, 35, 37, 38, 40, 42, 47, 57, 59, 61, 64, 78, 82, 83, 86, 91, 97, 99, 113, or 123 of SEQ ID NO: 22, as compared with the amino acid residues at those same positions in SEQ ID NO: 2. In one embodiment, the substitution mutation is S at position 2. In one embodiment, the substitution mutation is D at position 7. In one embodiment, the substitution mutation is A, S, or K at position 10. In one embodiment, the substitution mutation is R, D or Nat position 12. In one embodiment, the substitution mutation is R at position 15. In one embodiment, the substitution mutation is R at position 17. In one embodiment, the substitution mutation is D at position 20. In one embodiment, the substitution mutation is F at position 22. In one embodiment, the substitution mutation is A at position 35. In one embodiment, the substitution mutation is A, T or S at position 37. In one embodiment, the substitution mutation is A or S at position 38. In one embodiment, the substitution mutation is S at position 40. In one embodiment, the substitution mutation is K, E or G at position 42. In one embodiment, the substitution mutation is S at position 47. In one embodiment, the substitution mutation is D at position 57. In one embodiment, the substitution mutation is Y at position 59. In one embodiment, the substitution mutation is F at position 61. In one embodiment, the substitution mutation is S at position 64. In one embodiment, the substitution mutation is L at position 78. In one embodiment, the substitution mutation is T at position 82. In one embodiment, the substitution mutation is N or K at position 83. In one embodiment, the substitution mutation is D or S at position 86. In one embodiment, the substitution mutation is A or S at position 91. In one embodiment, the substitution mutation is I at position 97. In one embodiment, the substitution mutation is M at position 99. In one embodiment, the substitution mutation is D or I at position 113. In one embodiment, the substitution mutation is D at position 123.

In some embodiments of the above recombinant Ara h 6 variants, the Ara h 6 variants further comprise 2, 3, 4, 5, 6, 7, 8, 9, 10, 11, 12, 13, 14, 15, 16, 17, 18, 19, 20, 21, 22, 23, 24, 25, 26, or 27 substitution mutations at positions selected from positions 2, 7, 10, 12, 15, 17, 20, 22, 35, 37, 38, 40, 42, 47, 57, 59, 61, 64, 78, 82, 83, 86, 91, 97, 99, 113, or 123 of SEQ ID NO: 22, as compared with the amino acid residues at those same positions in SEQ ID NO: 2.

In one embodiment, the recombinant Ara h 6 variant polypeptide comprises one or more amino acid substitutions, deletions, insertions, or any combination thereof, that are located at one or more of positions 2, 3, 5, 7, 8, 10, 12, 16, 19, 22, 24, 33, 37, 38, 40, 41, 42, 45, 46, 47, 63, 74, 78, 81, 82, 83, 86, 89, 90, 97, 98, 99, 106, 108, 109, 110, 113, 114, 116, or 118 of SEQ ID NO: 109, as compared with the amino acid residues at those same positions in SEQ ID NO: 2.

In one embodiment, the one or more amino acid substitutions comprise one or more of: S at position 2; D, or S at position 3; D at position 5; D at position 7; A, or S at position 8; A, S, or K at position 10; R, D, or N at position 12; S, or D at position 16; Q, L, or R at position 19; F at position 22; D at position 24; Q, or K at position 33; A, T, or S at position 37; A, or S at position 38; S at position 40; D at position 41; K, E, or G at position 42; A, or Q at position 45; S, G, or R at position 46; S at position 47; R at position 74; L at position 78; A, or R at position 81; T at position 82; N, or K at position 83; D, or S at position 86; N, R, or G at position 89; D at position 90; I at position 97; D, or L at position 98; M at position 99; K, or H at position 106; P, E, or D at position 108; E, or S at position 110; D, or I at position 113; D, H, A, or G at position 114; K, or M at position 116; or R, or T at position 118.

In one embodiment, the substitution mutation is any amino acid at position 63. In one embodiment, the substitution mutation is any amino acid at position 109.

In one embodiment, the substitution mutation is S at position 2. In one embodiment, the substitution mutation is D or S at position 3. In one embodiment, the substitution mutation is D at position 5. In one embodiment, the substitution mutation is D at position 7. In one embodiment, the substitution mutation is A or S at position 8. In one embodiment, the substitution mutation is A, S, or K at position 10. In one embodiment, the substitution mutation is R, D or N at position 12. In one embodiment, the substitution mutation is S or D at position 16. In one embodiment, the substitution mutation is Q, L or R at position 19. In one embodiment, the substitution mutation is F at position 22. In one embodiment, the substitution mutation is D at position 24. In one embodiment, the substitution mutation is Q or K at position 33. In one embodiment, the substitution mutation is A, T or S at position 37. In one embodiment, the substitution mutation is A or S at position 38. In one embodiment, the substitution mutation Is S at position 40. In one embodiment, the substitution mutation is D at position 41. In one embodiment, the substitution mutation is K, E or G at position 42. In one embodiment, the substitution mutation is A or Q at position 45. In one embodiment, the substitution mutation is S, G or R at position 46. In one embodiment, the substitution mutation is S at position 47. In one embodiment, the substitution mutation is R at position 74. In one embodiment, the substitution mutation is L at position 78. In one embodiment, the substitution mutation is A or R at position 81. In one embodiment, the substitution mutation is T at position 82. In one embodiment, the substitution mutation is N or K at position 83. In one embodiment, the substitution mutation is D or S at position 86. In one embodiment, the substitution mutation is N, R, or G at position 89. In one embodiment, the substitution mutation is D at position 90. In one embodiment, the substitution mutation is I at position 97. In one embodiment, the substitution mutation is D or L at position 98. In one embodiment, the substitution mutation is M at position 99. In one embodiment, the substitution mutation is K or H at position 106. In one embodiment, the substitution mutation is P, E or D at position 108. In one embodiment, the substitution mutation is E or S at position 110. In one embodiment, the substitution mutation is D or I at position 113. In one embodiment, the substitution mutation is D, H, A or G at position 114. In one embodiment, the substitution mutation is K or M at position 116. In one embodiment, the substitution mutation is R or T at position 118.

In some embodiments of the above recombinant Ara h 6 variants, the Ara h 6 variants comprise at least 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, or 40 substitution mutations at positions selected from positions 2, 3, 5, 7, 8, 10, 12, 16, 19, 22, 24, 33, 37, 38, 40, 41, 42, 45, 46, 47, 63, 74, 78, 81, 82, 83, 86, 89, 90, 97, 98, 99, 106, 108, 109, 110, 113, 114, 116, or 118 of SEQ ID NO: 109, as compared with the amino acid residues at those same positions in SEQ ID NO: 2.

In one embodiment, the recombinant Ara h 6 variant polypeptide comprises amino acid substitutions, deletions, insertions, or any combination thereof, that are located at positions 3, 5, 8, 19, 45, 46, 89, 98, 110, 114, 116, and 118 of SEQ ID NO: 109, as compared with the amino acid residues at those same positions in SEQ ID NO: 2.

In one embodiment, the one or more amino acid substitutions comprise one or more of: D, or S at position 3; D at position 5; A, or S at position 8; Q, L, or R at position 19; A, or Q at position 45; S, G, or R at position 46; N, R, or G at position 89; D, or L at position 98; E, or S at position 110; D, H, A, or G at position 114; K, or M at position 116; or R, or T at position 118.

In one embodiment, the recombinant Ara h 6 variant polypeptide comprises the amino acid sequence set forth in SEQ ID NO: 109, wherein the variant comprises substitutions, deletions, insertions, or any combination thereof, that are located at one or more of positions 3, 5, 8, 19, 45, 46, 86, 89, 90, 98, 106, 108, 110, 114, 116, or 118 of SEQ ID NO: 109, as compared with the amino acid residues at those same positions in SEQ ID NO: 2.

In some embodiments of the above recombinant Ara h 6 variants, the Ara h 6 variants comprise at least 2, 3, 4, 5, 6, 7, 8, 9, 10, 11, 12, 13, 14, 15, or 16 substitution mutations at positions selected from positions 3, 5, 8, 19, 45, 46, 86, 89, 90, 98, 106, 108, 110, 114, 116, or 118 of SEQ ID NO: 110, as compared with the amino acid residues at those same positions in SEQ ID NO: 2.

In some embodiments, the recombinant Ara h 6 variant further comprises amino acid substitutions, deletions, insertions, or any combination thereof, at one or more of positions 2, 7, 10, 12, 15, 16, 17, 20, 22, 24, 28, 33, 35, 37, 38, 40, 41, 42, 47, 57, 59, 61, 63, 64, 74, 78, 81, 82, 83, 86, 90, 91, 97, 99, 106, 108, 109, 113, or 123 of SEQ ID NO: 109, as compared with the amino acid residues at those same positions in SEQ ID NO: 2.

In some embodiments, the one or more amino acid substitutions comprise one or more of: S at position 2; D at position 7; A, S, or K at position 10; R, D, or N at position 12; R at position 15; S, or D at position 16; R at position 17; D at position 20; F at position 22; D at position 24; S at position 28; Q, or K at position 33; A at position 35; A, T, or S at position 37; A, or S at position 38; S at position 40; D at position 41; K, E, or G at position 42; S at position 47; D at position 57; Y at position 59; F at position 61; S at position 64; R at position 74; L at position 78; A, or R at position 81; T at position 82; N, or K at position 83; S, or D at position 86 D at position 90; A, or S at position 91; I at position 97; M at position 99; K, or H at position 106; P, E, or D at position 108; D, or I at position 113; and D at position 123.

In one embodiment, the substitution mutation is any amino acid at position 63. In one embodiment, the substitution mutation is any amino acid at position 109.

In some embodiments of the above recombinant Ara h 6 variants, the Ara h 6 variants further comprise additional substitutions, deletions, insertions, or any combination thereof at one or more of positions 2, 7, 10, 12, 15, 16, 17, 20, 22, 24, 28, 33, 35, 37, 38, 40, 41, 42, 47, 57, 59, 61, 64, 74, 78, 81, 82, 83, 91, 97, 99, 113, or 123 of SEQ ID NO: 109, as compared with the amino acid residues at those same positions in SEQ ID NO: 2. In one embodiment, the substitution mutation is S at position 2. In one embodiment, the substitution mutation is D at position 7. In one embodiment, the substitution mutation is A, S, or K at position 10. In one embodiment, the substitution mutation is R, D or N at position 12. In one embodiment, the substitution mutation is R at position 15. In one embodiment, the substitution mutation is S or D at position 16. In one embodiment, the substitution mutation is R at position 17. In one embodiment, the substitution mutation is D at position 20. In one embodiment, the substitution mutation is F at position 22. In one embodiment, the substitution mutation is D at position 24. In one embodiment, the substitution mutation is S at position 28. In one embodiment, the substitution mutation is Q or K at position 33. In one embodiment, the substitution mutation is A at position 35. In one embodiment, the substitution mutation is A, T or S at position 37. In one embodiment, the substitution mutation is A or S at position 38. In one embodiment, the substitution mutation is S at position 40. In one embodiment, the substitution mutation is D at position 41. In one embodiment, the substitution mutation is K, E or G at position 42. In one embodiment, the substitution mutation is S at position 47. In one embodiment, the substitution mutation is D at position 57. In one embodiment, the substitution mutation is Y at position 59. In one embodiment, the substitution mutation is F at position 61. In one embodiment, the substitution mutation is S at position 64. In one embodiment, the substitution mutation is R at position 74. In one embodiment, the substitution mutation is L at position 78. In one embodiment, the substitution mutation is A or R at position 81. In one embodiment, the substitution mutation is T at position 82. In one embodiment, the substitution mutation is N or K at position 83. In one embodiment, the substitution mutation is A or S at position 91. In one embodiment, the substitution mutation is I at position 97. In one embodiment, the substitution mutation is M at position 99. In one embodiment, the substitution mutation is D or I at position 113. In one embodiment, the substitution mutation is D at position 123.

In some embodiments of the above recombinant Ara h 6 variants, the Ara h 6 variants comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, or 33 substitution mutations at positions selected from positions 2, 7, 10, 12, 15, 16, 17, 20, 22, 24, 28, 33, 35, 37, 38, 40, 41, 42, 47, 57, 59, 61, 64, 74, 78, 81, 82, 83, 91, 97, 99, 113, or 123 of SEQ ID NO: 109, as compared with the amino acid residues at those same positions in SEQ ID NO: 2.

In one embodiment, the recombinant Ara h 6 variant polypeptide comprises the amino acid sequence set forth in SEQ ID NO: 109, wherein the variant comprises substitutions, deletions, insertions, or any combination thereof, that are located at one or more of positions 2, 3, 5, 7, 8, 10, 12, 15, 16, 17, 19, 20, 22, 24, 28, 33, 35, 37, 38, 40, 41, 42, 45, 46, 47, 57, 59, 61, 64, 74, 78, 81, 82, 83, 86, 89, 90, 91, 97, 98, 99, 106, 108, 110, 113, 114, 116, 118 or 123 of SEQ ID NO: 109, as compared with the amino acid residues at those same positions in SEQ ID NO: 2. In one embodiment, the substitution mutation is S at position 2. In one embodiment, the substitution mutation is D or S at position 3. In one embodiment, the substitution mutation is D at position 5. In one embodiment, the substitution mutation is D at position 7. In one embodiment, the substitution mutation is A or S at position 8. In one embodiment, the substitution mutation is A, S, or K at position 10. In one embodiment, the substitution mutation is R, D or N at position 12. In one embodiment, the substitution mutation is R at position 15. In one embodiment, the substitution mutation is S or D at position 16. In one embodiment, the substitution mutation is R at position 17. In one embodiment, the substitution mutation is Q, L or R at position 19. In one embodiment, the substitution mutation is D at position 20. In one embodiment, the substitution mutation is F at position 22. In one embodiment, the substitution mutation is D at position 24. In one embodiment, the substitution mutation is S at position 28. In one embodiment, the substitution mutation is Q or K at position 33. In one embodiment, the substitution mutation is A at position 35. In one embodiment, the substitution mutation is A, T or S at position 37. In one embodiment, the substitution mutation is A or S at position 38. In one embodiment, the substitution mutation is S at position 40. In one embodiment, the substitution mutation is D at position 41. In one embodiment, the substitution mutation is K, E or G at position 42. In one embodiment, the substitution mutation is A or Q at position 45. In one embodiment, the substitution mutation is S, G or R at position 46. In one embodiment, the substitution mutation is S at position 47. In one embodiment, the substitution mutation is D at position 57. In one embodiment, the substitution mutation is Y at position 59. In one embodiment, the substitution mutation is F at position 61. In one embodiment, the substitution mutation is S at position 64. In one embodiment, the substitution mutation is R at position 74. In one embodiment, the substitution mutation is L at position 78. In one embodiment, the substitution mutation is A or R at position 81. In one embodiment, the substitution mutation is T at position 82. In one embodiment, the substitution mutation is N or K at position 83. In one embodiment, the substitution mutation is D or S at position 86. In one embodiment, the substitution mutation is N, R, or G at position 89. In one embodiment, the substitution mutation is D at position 90. In one embodiment, the substitution mutation is A or S at position 91. In one embodiment, the substitution mutation is I at position 97. In one embodiment, the substitution mutation is D or L at position 98. In one embodiment, the substitution mutation is M at position 99. In one embodiment, the substitution mutation is K or H at position 106. In one embodiment, the substitution mutation is P, E or D at position 108. In one embodiment, the substitution mutation is E or S at position 110. In one embodiment, the substitution mutation is D or I at position 113. In one embodiment, the substitution mutation is D, H, A or G at position 114. In one embodiment, the substitution mutation is K or M at position 116. In one embodiment, the substitution mutation is R or T at position 118. In one embodiment, the substitution mutation is D at position 123.

In some embodiments of the above recombinant Ara h 6 variants, the Ara h 6 variants comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, or 49 substitution mutations at positions selected from positions 2, 3, 5, 7, 8, 10, 12, 15, 16, 17, 19, 20, 22, 24, 28, 33, 35, 37, 38, 40, 41, 42, 45, 46, 47, 57, 59, 61, 64, 74, 78, 81, 82, 83, 86, 89, 90, 91, 97, 98, 99, 106, 108, 110, 113, 114, 116, 118 or 123 of SEQ ID NO: 109, as compared with the amino acid residues at those same positions in SEQ ID NO: 2.

In some embodiments of the above recombinant Ara h 6 variants, the Ara h 6 variants comprise 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 34, 35, 36, 37, 38, 39, 40, 41, 42, 43, 44, 45, 46, 47, 48, 49, 50 or 51 substitution mutations at positions selected from positions 2, 3, 5, 7, 8, 10, 12, 15, 16, 17, 19, 20, 22, 24, 28, 33, 35, 37, 38, 40, 41, 42, 45, 46, 47, 57, 59, 61, 63, 64, 74, 78, 81, 82, 83, 86, 89, 90, 91, 97, 98, 99, 106, 108, 109, 110, 113, 114, 116, 118 or 123 of SEQ ID NO: 109, as compared with the amino acid residues at those same positions in SEQ ID NO: 2.

In some embodiments, the recombinant Ara h 6 variant polypeptide comprises between 2-51 substitutions, deletions, insertions, or any combination thereof, e.g., 2, 3, 4, 5, 6, 7, 8, 9, 10, 11, 12, 13, 14, 15, 16, 17, 18, 19, 20, 21, 22, 23, 24, 25, 26, 27, 28, 29, 30, 31, 32, 33, 34, 35, 36, 37, 38, 39, 40, 41, 42, 43, 44, 45, 46, 47, 48, 49, 50, 51 or any range therebetween.

In some embodiments of the above recombinant Ara h 6 variants, the Ara h 6 variants comprise at least one or more amino acid substitutions, deletions, insertions, or any combination thereof, that are located at one or more of positions 15, 16, 17, 19, 20, 22, 24, 28, 57, 59, 61, 64, 74, 78, 81, 82, 83, 86, 98, or 116 of SEQ ID NO: 109, as compared with the amino acid residues at those same positions in SEQ ID NO: 2.

In some embodiments, the variant comprises the amino acid sequence as set forth in any of SEQ ID NOs: 3-21.

In some embodiments, the variant comprises the amino acid sequence as set forth in any of SEQ ID NOs: 24-108.

In some embodiments of the above recombinant Ara h 6 variants, the Ara h 6 variants comprise the amino acid sequence set forth in any of SEQ ID NOs: 3-21 or comprise an amino acid sequence having at least 80% identity with the amino acid sequences set forth in any of SEQ ID NOs: 3-21.

In some embodiments of the above recombinant Ara h 6 variants, the Ara h 6 variants comprise the amino acid sequence set forth in any of SEQ ID NOs: 3-21 or comprise an amino acid sequence having at least 77% identity with the amino acid sequences set forth in any of SEQ ID NOs: 3-21.

In some embodiments of the above recombinant Ara h 6 variants, the Ara h 6 variants comprise the amino acid sequence set forth in any of SEQ ID NOs: 3-21 or SEQ ID NOs: 24-108.

In some embodiments of the above recombinant Ara h 6 variants, the Ara h 6 variants comprise an amino acid sequence that is at least 70%, at least 75%, at least 77%, at least 80%, at least 85%, at least 90%, at least 95%, or at least 99% identical to the amino acid sequence set forth in any of SEQ ID NOs: 3-21 or SEQ ID NOs: 24-108.

In some embodiments of the above recombinant Ara h 6 variants, the Ara h 6 variants comprise the amino acid sequence set forth in any of SEQ ID NOs: 24-108 or comprise an amino acid sequence having at least 80% identity with the amino acid sequences set forth in any of SEQ ID NOs: 24-108.

In some embodiments of the above recombinant Ara h 6 variants, the Ara h 6 variants comprise the amino acid sequence set forth in any of SEQ ID NOs: 24-108 or comprise an amino acid sequence having at least 77% identity with the amino acid sequences set forth in any of SEQ ID NOs: 24-108.

In some embodiments, the recombinant Ara h 6 variant comprises the amino acid sequence set forth in SEQ ID NO: 9. In some embodiments, the recombinant Ara h 6 variant comprises the amino acid sequence set forth in SEQ ID NO: 46. In some embodiments, the recombinant Ara h 6 variant comprises the amino acid sequence set forth in SEQ ID NO: 76. In some embodiments, the recombinant Ara h 6 variant comprises the amino acid sequence set forth in SEQ ID NO: 80. In some embodiments, the recombinant Ara h 6 variant comprises the amino acid sequence set forth in SEQ ID NO: 82. In some embodiments, the recombinant Ara h 6 variant comprises the amino acid sequence set forth in SEQ ID NO: 101. In some embodiments, the recombinant Ara h 6 variant comprises the amino acid sequence set forth in SEQ ID NO: 108.

In some embodiments of the above recombinant Ara h 6 variants, basophile degranulation release induced by the variants is at least 10-fold lower compared with that induced by an Ara h 6 wild-type polypeptide.

In some embodiments of the above recombinant Ara h 6 variants, the variant has a binding EC50 or KD to at least one patient-derived-Ara h 6 IgE, that is reduced 50% or more as compared with that of an Ara h 6 wild-type polypeptide.

In some embodiments, the above recombinant Ara h 6 variants comprise one or more amino acid substitutions, deletions, insertions, or any combination thereof that are located within at least a single epitope recognized by an anti-Ara h 6 antibody.

In some embodiments, the Ara h 6 epitope comprises a linear epitope (L1) comprising amino acids at positions 2-14 of SEQ ID NO: 2. In some embodiments, the Ara h 6 epitope comprises a linear epitope (L2) comprising amino acids at positions 36-54 of SEQ ID NO: 2. In some embodiments, the Ara h 6 epitope comprises a linear epitope (L3) comprising amino acids at positions 76-90 of SEQ ID NO: 2. In some embodiments, the Ara h 6 epitope comprises a linear epitope (L4) comprising amino acids at positions 92-102 of SEQ ID NO: 2. In some embodiments, the Ara h 6 epitope comprises a linear epitope (L5) comprising amino acids at positions 104-118 of SEQ ID NO: 2.

In some embodiments, the Ara h 6 epitope comprises a conformational epitope (C1) comprising amino acids at positions 16, 19, 74, and 81 of SEQ ID NO: 2. In some embodiments, the Ara h 6 epitope comprises a conformational epitope (C2) comprising amino acids at positions 19, 22, and 24 of SEQ ID NO: 2. In some embodiments, the Ara h 6 epitope comprises a conformational epitope (C3) comprising amino acids at positions 33 and 106 of SEQ ID NO: 2. In some embodiments, the Ara h 6 epitope comprises a conformational epitope (C4) comprising amino acids at positions 63, 108, and 109 of SEQ ID NO: 2. In some embodiments, the Ara h 6 epitope comprises a conformational epitope (C5) comprising amino acids at positions 114 and 116 of SEQ ID NO: 2.

In some embodiments of the above recombinant Ara h 6 variants, the Ara h 6 variant comprise at least one, e.g., at least two or more, amino acid substitution, deletion, insertion, or any combination thereof located within at least one epitope. In some embodiments of the above recombinant Ara h 6 variants, the Ara h 6 variant comprise at least two amino acid substitution, deletion, insertion, or any combination thereof located within at least one conformational epitope selected from C1, C2, C3, C4 or C5. In some embodiments of the above recombinant Ara h 6 variants, the Ara h 6 variant comprise at least two amino acid substitutions, deletions, insertions, or any combination thereof located within at least two conformational epitopes selected from C1, C2, C3, C4 or C5. In some embodiments, conformational epitope (C1) comprises amino acids located at positions 16, 19, 74, and 81 of SEQ ID NO: 2. In some embodiments, conformational epitope (C2) comprises amino acids located at positions 19, 22, and 24 of SEQ ID NO: 2. In some embodiments, conformational epitope (C3) comprises amino acids located at positions 33 and 106 of SEQ ID NO: 2. In some embodiments, conformational epitope (C4) comprises amino acids located at positions 63, 108, and 109 of SEQ ID NO: 2. In some embodiments, conformational epitope (C5) comprises amino acids located at positions 114 and 116 of SEQ ID NO: 2.

In some embodiments of the above recombinant Ara h 6 variants, the Ara h 6 variant comprises amino acid substitutions, deletions, insertions, or any combination thereof, that are located at positions 3, 5, 8, 19, 45, 46, 89, 98, 110, 114, 116, and 118 of SEQ ID NO: 109, as compared with the amino acid residues at those same positions in SEQ ID NO: 2. In some embodiments, the variant comprises an amino acid sequence that is at least 80% identical to the amino acid sequence as set forth in any of SEQ ID NOs: 46, 76, 80, 82, 101. In some embodiments, the variant comprises an amino acid sequence that is at least 77% identical to the amino acid sequence as set forth in any of SEQ ID NOs: 46, 76, 80, 82, 101. In some embodiments, the variant comprises an amino acid sequence that is identical to the amino acid sequence as set forth in any of SEQ ID NOs: 46, 76, 80, 82, 101. In some embodiments, the Ara h 6 variant comprises conformational epitopes recognized by an anti-Ara h 6 IgG antibody. In some embodiments, the Ara h 6 variant has reduced or abolished binding to an anti-Ara h 6 IgE antibody, e.g., within conformational epitopes. In some embodiments, reduced refers to at least 10-fold reduction in binding affinity of the variant as compared to the binding of the natural or WT Ara h 6 (SEQ ID NOs: 1 and 2, respectively).

In some embodiments of the above recombinant Ara h 6 variants, the Ara h 6 variant comprises amino acid substitutions at positions 3, 5, 8, 19, 35, 37, 38, 45, 46, 89, 90, 98, 108, 110, 114, 116, 118 of SEQ ID NO: 109, as compared with the amino acid residues at those same positions in SEQ ID NO: 2. In some embodiments of the above recombinant Ara h 6 variants, the Ara h 6 variant comprises an amino acid sequence that is at least 80% identical to the amino acid sequence as set forth in SEQ ID NO:46.

In some embodiments of the above recombinant Ara h 6 variants, the Ara h 6 variant comprises amino acid substitutions at positions 3, 5, 8, 19, 35, 37, 38, 45, 46, 86, 89, 90, 98, 106, 108, 110, 114, 116, 118 of SEQ ID NO: 109, as compared with the amino acid residues at those same positions in SEQ ID NO: 2. In some embodiments of the above recombinant Ara h 6 variants, the Ara h 6 variant comprises an amino acid sequence that is at least 80% identical to the amino acid sequence as set forth in SEQ ID NO: 76.

In some embodiments of the above recombinant Ara h 6 variants, the Ara h 6 variant comprises amino acid substitutions at positions 3, 5, 8, 19, 45, 46, 86, 89, 90, 98, 106, 108, 110, 114, 116, 118 of SEQ ID NO: 109, as compared with the amino acid residues at those same positions in SEQ ID NO: 2. In some embodiments of the above recombinant Ara h 6 variants, the Ara h 6 variant comprises an amino acid sequence that is at least 80% identical to the amino acid sequence as set forth in SEQ ID NO: 80.

In some embodiments of the above recombinant Ara h 6 variants, the Ara h 6 variant comprises amino acid substitutions at positions 3, 5, 8, 19, 37, 45, 46, 86, 89, 90, 98, 106, 108, 110, 114, 116, 118 of SEQ ID NO: 109, as compared with the amino acid residues at those same positions in SEQ ID NO: 2. In some embodiments of the above recombinant Ara h 6 variants, the Ara h 6 variant comprises an amino acid sequence that is at least 80% identical to the amino acid sequence as set forth in SEQ ID NO: 82.

In some embodiments of the above recombinant Ara h 6 variants, the Ara h 6 variant comprises amino acid substitutions at positions 3, 5, 8, 19, 45, 46, 86, 89, 98, 106, 110, 114, 116, 118 of SEQ ID NO: 109, as compared with the amino acid residues at those same positions in SEQ ID NO: 2. In some embodiments of the above recombinant Ara h 6 variants, the Ara h 6 variant comprises an amino acid sequence that is at least 80% identical to the amino acid sequence as set forth in SEQ ID NO: 101.

In some embodiments of the above recombinant Ara h 6 variants, the Ara h 6 variant comprises a methionine located upstream of the N terminus of the amino acid sequence (or protein sequences) described herein. In this embodiment, methionine thus forms the N terminus of the amino acid sequence. Such a methionine usually originates from the translation of the protein-encoding RNA, where it is encoded by the nucleic acid triplet ATG. Unless already present in the nucleic acid sequence, the ATG triplet can be attached to the 5′ end of a nucleic acid molecule by the method known to the skilled artisan.

In some embodiments of the Ara h 6 variants, the amino acid sequence set forth in any of SEQ ID NOs: 3-21 further comprises a methionine located upstream of the N terminus of the amino acid sequence set forth in any of SEQ ID NOs: 3-21. In some embodiments of the Ara h 6 variants, the amino acid sequence set forth in any of SEQ ID NOs: 24-108 further comprises a methionine located upstream of the N terminus of the amino acid sequence set forth in any of SEQ ID NOs: 24-108.

In some embodiments of the Ara h 6 variants, the amino acid sequence further comprises a label or tag (purification tag or stability tag) at its N-terminus or at its C-terminus. In some embodiments, the tag is chosen from a His tag, HA-tag, or other suitable tags known in the art.

In some embodiments, “SEQ ID NO:110” and “SEQ ID NO:109” are used herein interchangeably.

### Nucleotides, Vectors, and Host Cells

In one embodiment, the present disclosure provides an isolated nucleotide or modified nucleotide sequence encoding the recombinant Ara h 6 variant described herein in detail. In some embodiments, the nucleotide or modified nucleotide sequence is DNA or mRNA. In some embodiments of the nucleotide or modified nucleotide sequence, the mRNA comprises liquid nanoparticle (LNP)-formulated mRNA.

In one embodiment, the present disclosure provides an expression vector comprising the isolated nucleotide or modified nucleotide sequence described herein.

In one embodiment, the present disclosure provides a prokaryotic cell or a eukaryotic cell comprising the expression vector described herein. Ins some embodiments, the eukaryotic cell is a yeast cell, a fungi cell, a plant cell, or a mammalian cell.

In one embodiment, the present disclosure provides a composition comprising the recombinant Ara h 6 variant polypeptide described herein in detail.

The term “nucleotide”, “nucleotide sequence” or “nucleic acid molecule” as used herein is intended to include DNA molecules and RNA molecules or modified RNA molecules. A nucleic acid molecule may be single-stranded or double-stranded. In some embodiments, a nucleotide comprises a modified nucleotide. In some embodiments, a nucleotide comprises an mRNA. In some embodiments, a nucleotide comprises a modified mRNA. In some embodiments, a nucleotide comprises a modified mRNA, wherein the modified mRNA comprises a 5′-capped mRNA. In some embodiments, a modified mRNA comprises a molecule in which some of the nucleosides have been replaced by either naturally modified or synthetic nucleosides. In some embodiments, a modified nucleotide comprises a modified mRNA comprising a 5′-capped mRNA and wherein some of the nucleosides have been replaced by either naturally modified or synthetic nucleosides.

The term “isolated nucleotide” or “isolated nucleic acid molecule” as used herein refers to nucleic acids encoding the peanut allergen variants disclosed herein (e.g., Ara h 6 variants) in which the nucleotide sequences are essentially free of other genomic nucleotide sequences that naturally flank the nucleic acid in genomic DNA.

Disclosed herein, in one aspect is a nucleotide or nucleic acid sequence encoding the peanut allergen variants disclosed herein (e.g., Ara h 6 variants).

As used herein, the term “vector” refers to discrete elements that are used to introduce heterologous nucleic acids into cells for either expression or replication thereof. An expression vector includes vectors capable of expressing nucleic acids that are operatively linked with regulatory sequences, such as promoter regions, that are capable of affecting expression of such nucleic acids. Thus, an expression vector may refer to a DNA or RNA construct, such as a plasmid, a phage, recombinant virus, or other vector that, upon introduction into an appropriate host cell, results in expression of the nucleic acids. Appropriate expression vectors are well known to those of skill in the art and include those that are replicable in prokaryotic cells and/or eukaryotic cells, and those that remain episomal or those which integrate into the host cell genome.

Disclosed herein, in one aspect is an expression vector comprising the nucleic acid construct encoding the peanut allergen variants disclosed herein (e.g., Ara h 6 variants).

The term “recombinant host cell” (or simply “host cell”) as used herein refers to a cell into which a recombinant expression vector has been introduced. It should be understood that such terms are intended to refer not only to the particular subject cell but to the progeny of such a cell. Because certain modifications may occur in succeeding generations due to either mutation or environmental influences, such progeny may not, in fact, be identical to the parent cell, but are still included within the scope of the term “host cell” as used herein.

Disclosed herein, in one aspect is a host cell comprising an expression vector carrying the nucleic acid construct encoding the peanut allergen variants disclosed herein (e.g., Ara h 6 variants). In one embodiment, the cell or host cell is a prokaryotic cell or a eukaryotic cell. In one embodiment, the eukaryotic cell is a yeast cell, a fungi cell, an algae cell, a plant cell, or a mammalian cell. In some embodiments, the peanut allergen variants may be produced in bacteria, such as E. Coli. In some other embodiments, the peanut allergen variants may be produced in yeast or fungi, such as Saccharomyces cerevisiae, Aspergillus, Trichoderma or Pichia pastoris.

### Nucleic Acid Encoding Ara h 6 Variants

In one embodiment, provided herein are nucleic acid or modified nucleic acid molecules encoding a recombinant Ara h 6 variant polypeptide comprising an amino acid sequence that is at least 80% identical to the sequence set forth in SEQ ID NO:2, wherein the Ara h 6 variant comprises one or more amino acid substitutions, deletions, insertions, or any combination thereof that are located within a single epitope recognized by an anti-Ara h 6 antibodies.

In another embodiment, the nucleic acid or modified nucleic acid molecules encode a recombinant Ara h 6 variant comprising an amino acid sequence that is at least 80% identical to the sequence set forth in SEQ ID NO: 2, wherein the Ara h 6 variant comprises one or more amino acid substitutions, deletions, insertions, or any combination thereof that are located within at least two epitopes recognized by anti-Ara h 6 antibodies.

A skilled artisan would appreciate that percent identity (% identity) provides a number that describes how similar the query sequence is to the target sequence (i.e., how many amino acids in each sequence are identical). The higher the percent identity is, the more significant the match.

When used in relation to polypeptide (or protein) sequences, the term “identity” refers to the degree of identity between two or more polypeptide (or protein) sequences or fragments thereof. Typically, the degree of similarity between two or more polypeptide (or protein) sequences refers to the degree of similarity of the composition, order, or arrangement of two or more amino acids of the two or more polypeptides (or proteins).

In some embodiments, the variant Ara h 6 polypeptides comprise an amino acid sequence that is at least 70%, at least 75%, at least 77%, at least 80%, at least 85%, or at least 90% identical to the amino acid sequence SEQ ID NO:2 or a portion thereof disclosed herein, as determined using BlastP software of the National Center of Biotechnology Information (NCBI) using default parameters.

In some embodiments, the Ara h 6 variants described herein may encompass deletion, insertion, or amino acid substitution mutations. In one embodiment, the variant polypeptide comprises conservative substitutions, or deletions, insertions, or substitutions that do not significantly alter the three-dimensional structure of the polypeptide of interest described herein. In some embodiments, the deletion, insertion, or substitution does not alter the function of the polypeptide of interest disclosed herein. In some embodiments, the deletion, insertion, or substitution does not alter the potential to induce the immune system's response and generate desensitization to the peanut allergen.

In one embodiment, the nucleic acid or modified nucleic acid is DNA or mRNA. In one embodiment, the mRNA comprises a UTR, or the mRNA comprises a leader sequence, or the mRNA comprises a UTR and a leader sequence. In one embodiment, the UTR comprises a chimeric or novel sequence that may outperform a natural UTR sequence, promoting overall higher protein expression.

In one embodiment, the mRNA comprises an optimized sequence. As used herein, an “optimized sequence” encompasses an mRNA sequence comprising a computationally altered nucleotide sequence that facilitates higher expression levels in human cells, compared with the non-altered sequence, while maintaining characteristics that are favorable for in vitro transcription (IVT) and enzymatic capping.

In one embodiment, the nucleic acid or modified nucleic acid molecules disclosed herein encode an Ara h 6 variant comprising the amino acid sequence set forth in any one of SEQ ID NOs: 3-21 or comprises an amino acid sequence having at least 80% identity with the amino acid sequences set forth in any of SEQ ID NOs: 3-21.

In one embodiment, the nucleic acid or modified nucleic acid molecules disclosed herein encode an Ara h 6 variant comprising the amino acid sequence set forth in any one of SEQ ID NOs: 24-108 or comprises an amino acid sequence having at least 80% identity with the amino acid sequences set forth in any of SEQ ID NOs: 24-108.

In some embodiments the nucleic acid or modified nucleic acid molecule disclosed herein further comprise nucleic acid sequences encoding a label or tag (purification tag or stability tag) at 5′ or 3′ end. In some embodiments, the tag is chosen from a His tag, HA-tag, or other tags known in the art. In some impairments, the nucleic acid or modified nucleic acid molecule disclosed herein further comprises restriction endonuclease sequences.

### Methods of Production

In some embodiments, variant polypeptides disclosed herein can be produced using a cell free in-vitro translation system, as is well known in the art for example but not limited to methods reviewed in Dondapati et al. (2020) BioDrugs 34(3):327-348. In one embodiment, the present disclosure provides a method of producing a hypo-allergenic peanut allergen comprising Ara h 6 variants disclosed herein, the method comprising culturing cells comprising the expression vector described above under conditions to express the Ara h 6 variant. In one embodiment, the cell is a prokaryotic cell or a eukaryotic cell. In one embodiment, the eukaryotic cell is a yeast cell, a fungi cell, a plant cell, or a mammalian cell.

In some embodiments, the nucleic acid or modified nucleic acid molecules disclosed herein, is transcribed in an in vitro transcription system (IVT), wherein the transcribed nucleic acid or modified nucleic acid may then be used for immunotherapy by gene delivery, wherein administration of the mRNA results in the in-vivo production of a peanut allergen or peanut allergen variants.

In some embodiments of a method of production, the nucleic acid molecule encodes a variant Ara h 6 polypeptide comprising one or more amino acid substitutions, deletions, insertions, or any combination thereof that are located within a single epitope recognized by an anti-Ara h 6 antibody. In some embodiments, the nucleic acid comprises a modified nucleic acid encoding a variant Ara h 6 polypeptide comprising one or more amino acid mutations that are located within a single epitope recognized by an anti-Ara h 6 antibody.

Synthesis and capping of RNA molecules, either by chemical synthesis or by enzymatic processes such as bacteriophage RNA polymerases are well established methods in the art for mRNA production as described by Elain T. Schenborn Methods in Molecular Biology, Vol. 37: In Vitro Transcript/on and Translation Protocols pages 1-12 DOI: 10.1385/0-89603-288-4:1.

One skilled in the art would appreciate that other known IVT systems may be used to transcribe the nucleic acid or modified nucleic acid molecules described herein. In some embodiments, an mRNA molecule is transcribed in vitro using an IVT system.

Production of peanut allergen variants, Ara h 6 variants may comprise in vivo translation, wherein a transcribed mRNA is administered to a subject (in vivo translation).

In some embodiments, the nucleic acid or modified nucleic acid molecules disclosed herein, can be used to produce peanut allergen variant polypeptides in vivo, comprising administration of a nucleic acid or modified nucleic acid molecule by viral, nonviral or physical means such as liposome, cationic lipid, cationic polymer or hybrid lipid polymer systems, retroviral or DNA viral delivery e.g. lentiviral, foamyviral, adenoviral etc. sonoporation, electroporation, hydrodynamic delivery to a subject. In some embodiments, the nucleic acid molecules disclosed herein can be used to produce peanut allergen WT polypeptides in vivo, comprising administration of a nucleic acid molecule by viral, nonviral or physical means such as liposome, cationic lipid, cationic polymer or hybrid lipid polymer systems, retroviral or DNA viral delivery e.g. lentiviral, foamyviral, adenoviral etc. sonoporation, electroporation, hydrodynamic delivery to a subject. In vivo methods of administration of nucleic acid molecules, for example the mRNA molecules described herein encoding Ara h 6 variants, are well known in the art for example but not limited to methods reviewed in Jones et al., Overcoming Nonviral Gene Delivery Barriers: Perspective and Future. Mol. Pharmaceutics 2013, 10, 11, 4082-4098; Kamimura et al. Advances in Gene Delivery Systems. Pharmaceut Med. 25(5):293-306; and Nayerossadat et al., Viral and nonviral delivery systems for gene delivery. Adv Biomed Res 2012; 1:27, which are incorporated herein in full.

In some embodiments, a subject comprises a human subject. In certain embodiments, a subject comprises a baby, a child, an adolescent, a young adult, or a mature adult human. In some embodiments, a subject comprises a baby.

In some embodiments, a subject comprises one in need of inducing desensitization to peanuts. In some embodiments, a subject is allergic to peanuts. In some embodiments, a subject suffers from other food allergies. In some embodiments, a subject may be prone to develop peanut allergy.

### Methods of Use

In one embodiment, the present disclosure provides a method of inducing desensitization to peanuts in a subject allergic to peanuts, the method comprising administering to the subject a composition comprising the hypo-allergenic Ara h 6 variants disclosed herein, thereby increasing the ability to tolerate peanuts in the subject.

In one embodiment, the present disclosure provides a method of inducing immunomodulation of a response to peanuts in a subject allergic to peanuts, the method comprising administering to the subject a composition comprising the hypo-allergenic Ara h 6 variants disclosed herein, thereby increasing the ability to tolerate peanuts in the subject.

As used herein allergy desensitization to peanuts or desensitization to peanuts, also termed allergy immunotherapy, allergy immunomodulation, immunomodulation of a response to peanuts or allergen-specific immunotherapy, is a treatment aiming to reduce the severity of clinical reaction to peanuts and/or to increase the tolerated dose of peanuts and/or the long-term tolerance to peanuts. Peanut immunotherapy can be tested using methods known in the art, including a food challenge. Peanut immunotherapy may be partial, wherein the subject tolerates an increased amount of the food allergen compared to prior to treatment, but still reacts to higher doses of the food allergen; or the desensitization may be complete, wherein the patient tolerates all tested doses of the food allergen. In some embodiments, desensitization to peanuts comprises a reduced activation potential of basophils and/or mast cells compared to prior to treatment.

In some embodiments, “immunomodulation of a response to peanuts” comprises a reduced allergic response to peanuts. In some embodiments, a reduced allergic response to peanuts in a subject is reduced (decreased) relative to the allergic response to peanuts of the subject prior to treatment or as compared with an earlier timepoint in the course of treatment. In some embodiments, the reduced allergic response to peanuts provides for a 10%, 20%, 30%, 40%, 50%, 60%, 70%, 80%, 90%, 95%, or 99% decrease in the allergic response to peanuts. In some embodiments, the reduced allergic response to peanuts in a subject comprises a reduced activation potential of basophils and/or mast cells compared to prior to treatment.

As used herein “allergy immunomodulation” also termed “allergy desensitization”, “allergy immunotherapy”, or “allergen-specific immunotherapy”, is a treatment aiming to reduce the severity of clinical reaction to peanuts, or to increase the tolerated dose of peanuts. Peanut immunotherapy can be tested using methods known in the art, including a food challenge. Peanut immunotherapy may be partial, wherein the subject tolerates an increased amount of the food allergen compared to prior to treatment, but still reacts to higher doses of the food allergen; or the desensitization may be complete, wherein the patient tolerates all tested doses of the food allergen.

In some embodiments, the methods described herein comprise the use of adjuvant. “Adjuvant”, according to the present invention, refers to a compound or mixture that enhances the immune response to an antigen. An adjuvant may also serve as a tissue depot that slowly releases the antigen. Examples of adjuvants include, but are not limited to, monophosphoryl lipid A (MPL-A), MicroCrystalline Tyrosine (MCT), Calcium phosphate, complete Freund's adjuvant, incomplete Freund's adjuvant, saponin, mineral gels such as aluminum hydroxide, surface active substances such as lysolecithin, pluronic polyols, polyanions, peptides, Levamisol, CpG-DNA, oil or hydrocarbon emulsions, and potentially useful adjuvants such as BCG (bacille Calmette-Guerin) and Corynebacterium parvum. In some embodiments, Ara h 6 variants are adsorbed to the MCT and administered with or without MPL-A. Both MCT and MPL-A should improve the efficacy of allergy immunotherapy and may have a synergistic effect when combined. Specifically, the adjuvants' administration may decrease the number of injections needed, decrease the dose and result in enhanced production of protective IgG antibodies. In addition, MCT adsorption may improve the safety of the product due to depot effect and gradual release of the proteins.

In one embodiment, the present disclosure provides a method of inducing desensitization to peanuts in a subject allergic to peanuts, the method comprising administering to the subject a composition comprising nucleotide or modified nucleotide sequences encoding the recombinant hypo-allergenic Ara h 6 variants disclosed herein, thereby inducing desensitization to peanuts in the subject. In one embodiment, the present disclosure provides a method of inducing desensitization to peanuts in a subject allergic to peanuts, the method comprising administering to the subject a composition comprising nucleotide or modified nucleotide sequences encoding the recombinant hypo-allergenic Ara h 6 variants disclosed herein, thereby inducing desensitization to peanuts in the subject. In one embodiment, the above composition comprises bacteria carrying the nucleotide sequences. In one embodiment, the nucleotide sequences are in the form of DNA or RNA.

In one embodiment, the present disclosure provides a method of inducing immunomodulation of a response to peanuts in a subject allergic to peanuts, the method comprising administering to the subject a composition comprising nucleotide or modified nucleotide sequences encoding the recombinant hypo-allergenic Ara h 6 variants disclosed herein, thereby inducing immunomodulation of a response to peanuts in the subject. In one embodiment, the present disclosure provides a method of inducing immunomodulation of a response to peanuts in a subject allergic to peanuts, the method comprising administering to the subject a composition comprising nucleotide or modified nucleotide sequences encoding the recombinant hypo-allergenic Ara h 6 variants disclosed herein, thereby inducing immunomodulation of a response to peanuts in the subject. In one embodiment, the above composition comprises bacteria carrying the nucleotide sequences. In one embodiment, the nucleotide sequences are in the form of DNA or RNA.

In one embodiment, the composition in the above methods is administered orally. In another embodiment, the composition is administered by a route selected from sub-cutaneous, intra-muscular, intra-dermal, intra-nasal, sub-lingual, topical, rectal or inhalation. In one embodiment, the subject in the above methods is an infant. In one embodiment, the composition in the above methods comprises a milk formula or a baby food.

In one embodiment, the present disclosure provides a method of inducing desensitization to peanuts in a subject allergic to peanuts, the method comprising administering to the subject a composition comprising a nucleic acid molecule encoding a recombinant Ara h 6 polypeptide, thereby inducing desensitization to peanuts in the subject. In some embodiments, a nucleic acid molecule used in a method of inducing desensitization to peanuts in a subject allergic to peanuts, comprises a nucleic acid molecule encoding a WT recombinant Ara h 6 polypeptide. In some embodiments, a nucleic acid molecule used in a method of inducing desensitization to peanuts in a subject allergic to peanuts, comprises a nucleic acid molecule or a modified nucleic acid molecule encoding a variant recombinant Ara h 6 polypeptide comprising one or more amino acid substitution mutations that are located within a single epitope recognized by an anti-Ara h 6 antibody.

In one embodiment, the present disclosure provides a method of inducing desensitization to peanuts in a subject allergic to peanuts, the method comprising administering to the subject a composition comprising a nucleic acid or modified nucleic acid molecule encoding a recombinant hypo-allergenic Ara h 6 variant disclosed herein, thereby inducing desensitization to peanuts in the subject.

In one embodiment, the present disclosure provides a method of inducing immunomodulation of a response to peanuts in a subject allergic to peanuts, the method comprising administering to the subject a composition comprising a nucleic acid molecule encoding a recombinant Ara h 6 polypeptide, thereby inducing immunomodulation of a response to peanuts in the subject. In some embodiments, a nucleic acid molecule used in a method of inducing immunomodulation of a response to peanuts in a subject allergic to peanuts, comprises a nucleic acid molecule encoding a WT recombinant Ara h 6 polypeptide. In some embodiments, a nucleic acid molecule used in a method of inducing immunomodulation of a response to peanuts in a subject allergic to peanuts, comprises a nucleic acid molecule or a modified nucleic acid molecule encoding a variant recombinant Ara h 6 polypeptide comprising one or more amino acid substitution mutations that are located within a single epitope recognized by an anti-Ara h 6 antibody.

In one embodiment, the present disclosure provides a method of inducing immunomodulation of a response to peanuts in a subject allergic to peanuts, the method comprising administering to the subject a composition comprising a nucleic acid or modified nucleic acid molecule encoding a recombinant hypo-allergenic Ara h 6 variant disclosed herein, thereby inducing immunomodulation of a response to peanuts in the subject.

In some embodiments, the composition comprising the isolated nucleotide or modified nucleotide sequence encoding the recombinant Ara h 6 variant described herein is for use in inducing desensitization to peanuts in a subject allergic to peanuts. In some embodiments, the composition comprising the isolated nucleotide or modified nucleotide sequence encoding the recombinant Ara h 6 variant described herein is for use in inducing immunomodulation of a response to peanuts in a subject allergic to peanuts.

In some embodiments, the composition comprising the recombinant Ara h 6 variant polypeptide described herein is for use in inducing desensitization to peanuts in a subject allergic to peanuts. In some embodiments, the composition comprising the recombinant Ara h 6 variant polypeptide described herein is for use in inducing immunomodulation of a response to peanuts in a subject allergic to peanuts.

In one embodiment, the composition in the above methods comprises bacteria carrying the nucleic acid or modified nucleic acid molecules disclosed herein. In one embodiment, the nucleic acid or modified nucleic acid molecules are DNA or mRNA. Examples of DNA or mRNA have been described above.

In one embodiment, the composition in the above methods is administered orally. In another embodiment, the composition is administered by a route selected from sub-cutaneous, intra-muscular, intravenous, intra-nasal, sub-lingual, topical, rectal or inhalation. In one embodiment, the subject in the above methods is an infant.

As used herein, “nucleic acid composition” refers to a composition which includes a nucleic acid or nucleic acid molecule (e.g., a polynucleotide) encoding an allergen or derivative thereof (e.g., variants of Ara h 6 protein or polypeptide). In exemplary embodiments, a nucleic acid composition includes a ribonucleic (“RNA”) polynucleotide, ribonucleic acid (“RNA”) or ribonucleic acid (“RNA”) molecule. Such embodiments can be referred to as ribonucleic acid (“RNA”) compositions. In some embodiments, a nucleic acid composition includes a messenger RNA (“mRNA”) polynucleotide, messenger RNA (“mRNA”) or messenger RNA (“mRNA”) molecule as described herein. Such embodiments can be referred to as messenger RNA (“mRNA”) compositions. Said compositions may comprise other substances and molecules which are required, or which are advantageous when said composition is administered to an individual (e.g., pharmaceutical excipients).

In one embodiment, the RNA composition comprises RNA sequence encoding the allergen. This RNA sequence can be the sequence of the allergen or can be adapted with respect to its codon usage. Adaption of codon usage can increase translation efficacy and half-life of the RNA. In one embodiment, a poly A tail comprising at least 30 adenosine residues is attached to the 3′ end of the RNA to increase the half-life of the RNA. In one embodiment, the 5′ end of the RNA is capped with a modified ribonucleotide with the structure m7G(5′)ppp(5′)N(cap 0 structure) or a derivative thereof which can be incorporated during RNA synthesis or can be enzymatically engineered after RNA transcription by using Vaccinia Virus Capping Enzyme (VCE, consisting of mRNA triphosphatase, guanylyl-transferase and guanine-7-methytransferase), which catalyzes the construction of N7-monomethylated cap 0 structures. Cap 0 structure plays a crucial role in maintaining the stability and translational efficacy of the RNA composition. The 5′ cap of the RNA composition can be further modified by a 2′-O-Methyltransferase which results in the generation of a cap 1 structure (m7Gppp[m2′-O]N), which further increases translation efficacy. The composition or formulation according to the present invention can further include an adjuvant.

In one embodiment, the Ara h 6 variant and/or the nucleic acid or modified nucleic acid molecule encoding the recombinant Ara h 6 variant disclosed herein are combined for inducing desensitization to peanuts in a subject allergic to peanuts. In one embodiment, several variants and/or several nucleic acid or modified nucleic acid molecules encoding the recombinant Ara h 6 variants disclosed herein are combined for inducing desensitization to peanuts in a subject allergic to peanuts. In one embodiment, the Ara h 6 variant and/or the nucleic acid or modified nucleic acid molecule encoding the recombinant Ara h 6 variant disclosed herein are combined with other compositions and/or treatments, e.g., other Ara h x allergens and variants thereof (WT and/or mutated), for inducing desensitization to peanuts in a subject allergic to peanuts.

### Plants and Products

In one embodiment, the present disclosure provides a genetically modified peanut plant, the peanut plant comprising peanuts expressing the Ara h 6 variants disclosed herein.

In one embodiment, the Ara h 6 variants, expressed in the above genetically modified peanut plant are expressed from a heterologous nucleic acid.

In one embodiment, the Ara h 6 variants expressed in the above genetically modified peanut plant are endogenously expressed from a genetically modified chromosome.

In some embodiments of the above genetically modified peanut plant, expression of endogenous wild-type Ara h 6 allergen is reduced compared with a non-genetically modified peanut plant.

In some embodiments, the reduced expression of endogenous wild-type Ara h 6 allergen in the genetically modified peanut plant is compared to the amount of endogenous wild-type Ara h 6 allergen in a corresponding non-genetically modified peanut plant. In some embodiments, the reduced expression of endogenous wild-type Ara h 6 allergen in the genetically modified peanut plant comprises at least 50%, at least 60%, at least 70%, at least 75%, 80 at least %, at least 85%, at least 90%, at least 95%, or up to 99% less expression when compared to the endogenous wild-type Ara h 6 allergen expression in a corresponding non-genetically modified peanut plant.

In some embodiments of the above genetically modified peanut plant, the modified plant further expresses at least one RNA silencing molecule that (i) reduces expression of the endogenous Ara h 6 allergen, and (ii) does not reduce the expression of the Ara h 6 variant.

In some embodiments of the above genetically modified peanut plant, the modified plant further expresses a DNA editing system directed towards reducing expression of the endogenous Ara h 6 allergen.

In one embodiment, the present disclosure provides a processed food product comprising the Ara h 6 variants disclosed herein.

In one embodiment, the above processed food product comprises a reduced amount of endogenous wild-type peanut Ara h 6 allergen. In some embodiments, the reduced amount of endogenous wild-type Ara h 6 allergen is compared to the amount of endogenous wild-type Ara h 6 allergen in a corresponding processed food product which does not comprise the Ara h 6 variant described herein in detail. In some embodiments, the reduced amount of endogenous wild-type Ara h 6 allergen comprises at least 50%, at least 60%, at least 70%, at least 75%, at least %80, at least 85%, at least 90%, at least 95%, or up to 99% less endogenous wild-type Ara h 6 allergen, when compared to the endogenous peanut Ara h 6 allergen in a corresponding processed food product which does not comprise the Ara h 6 variant described herein in detail.

In one embodiment, the above processed food product comprises a peanut harvested from the genetically modified plant described above.

The terms “comprises”, “comprising”, “includes”, “including”, “having” and their conjugates mean “including but not limited to”.

As used herein, the singular form “a”, “an” and “the” include plural references unless the context clearly dictates otherwise. For example, the term “A recombinant Ara h 6 variant” may include a plurality of variants, including mixtures thereof. Similarly, the term “An isolated nucleotide or modified nucleotide sequence encoding the recombinant Ara h 6 variant” may include a plurality of nucleotide or modified nucleotide sequences, including mixtures thereof.

All numeric values are herein assumed to be modified by the term “about”. The term “about” as used herein indicates values that may deviate up to 1%, more specifically 5%, more specifically 10%, more specifically 15%, and in some cases up to 20% higher or lower than the value referred to, the deviation range including integer values, and, if applicable, non-integer values as well, constituting a continuous range.

The word “exemplary” is used herein to mean “serving as an example, instance or illustration”. Any embodiment described as “exemplary” is not necessarily to be construed as preferred or advantageous over other embodiments and/or to exclude the incorporation of features from other embodiments.

Throughout this application, various embodiments of Ara h 6 variants, and mutation and/or epitope positions thereof may be presented in a range format. It should be understood that the description in range format is merely for convenience and brevity and should not be construed as an inflexible limitation on the scope of Ara h 6 variants and mutation and/or epitope positions thereof. Accordingly, the description of a range should be considered to have specifically disclosed all the possible sub ranges as well as individual numerical values within that range. For example, description of a range such as from 1 to 6 should be considered to have specifically disclosed sub ranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

Whenever a numerical range is indicated herein, it is meant to include any cited numeral (fractional or integral) within the indicated range. The phrases “ranging/ranges between” a first indicate number and a second indicate number and “ranging/ranges from” a first indicate number “to” a second indicate number are used herein interchangeably and are meant to include the first and second indicated numbers and all the fractional and integral numerals there between.

### EXAMPLES

### Example 1: Materials and Methods

### Peptide Microarray Assay

To determine Ara h 6 epitopes, a Celluspot™ peptide microarray-based immunoassay (Intavis, Cologne, Germany) was performed (Winkler, Dirk F H, Peptide microarrays. Humana Press, 2009). The peptides, of 15 amino-acids in length with an offset of 4 amino-acids, derived from the primary sequence of peanut allergens Ara h 1 (uniprot entry P43238 positions 25-626), Ara h 2 (uniprot entry Q6PSU2), Ara h 3 (uniprot entry 082580), Ara h 6 (uniprot entry A5Z1R0 positions 13-147) and Ara h 8 (uniprot entry Q6VT83), were synthesized and spotted on the microarray in duplicates. The slides were rinsed with a blocking buffer (150 mM NaCl, 0.05% Tween, 2.5% skim milk, 50 mM Tris pH7.5) for overnight at 4 °C. Then, the slides were washed and incubated with plasma in a blocking buffer incubated for 4 hr at 4 °C on a rotator. For detection, the slides were incubated with 3 ml of horseradish peroxidase (HRP)-tagged goat-anti-human IgE (abcam, Cambridge, United Kingdom), or diluted 1:10,000 in a blocking buffer for 2 hours at 25 °C on a rotator. After washes, femtogram HRP Substrate kit [Azure Biosystem, Dublin, California] was added and chemiluminescence was read via ChemiDoc [BioRad, Hercules, CA]. Peptide array images were processed by an in-house python script that detects peptide spots, normalizes their intensities, and reports any series of at least two overlapping spots showing across the duplicate a mean signal that is higher than two standard deviations from the slide mean.

Generation of Human scFv Phage Display Library

Whole blood samples of 5-20 ml were taken from clinically diagnosed peanut allergy patients using Heparin or EDTA treated tubes (BD). Peripheral blood mononuclear cells (PBMC) were extracted from blood samples using Sepmate tubes (STEMCELL) according to the manufacturer's instructions. RNA was purified from 5-15×10⁶ PBMC using the RNAeasy extraction kits (Qiagen; Hilden, Germany) and cDNA was prepared from 1-5 μg RNA (depending on the amount of RNA obtained).

The entire cDNA reaction was divided into PCR reactions to amplify the antibodies hyper-variable domain of each patient's variable genes. Light chains were amplified using gene sub-family specific forward primers carrying an unstructured, non-specific overhang followed by a NotI restriction site and reverse primers specific for the IGLK and IGLL isotypes carrying homology to the 5′ portion of an unstructured linker. Heavy chains were amplified using gene sub-family specific forward primers carrying homology to the 3′ portion of an unstructured linker and reverse primers specific for IGHG and IGHE genes carrying an unstructured, non-specific overhang followed by a NcoI restriction site. Primers were adapted from “Phage display: Methods and Protocols” (2018) Hust M and List T eds. Springer Protocols. PCR 50 μl reactions were performed with Phusion hot start Taq Polymerase kit, 200 μM dNPT, 2% DMSO, 1.25M Betaine, 1-5 μg cDNA and 0.5 μM each primer. Reactions were performed using the following PCR program: 3 min at 98 °C, 30 cycles of 98 °C 20 sec+60 °C 60 sec+72 °C 45 sec, and a final elongation stage of 72 °C for 10 min.

PCR products of each family (VHγ, VHε, VLκ and VLλ) were combined, each pool was concentrated by ethanol precipitation, ran on a 1% agarose gel, extracted using gel extraction kit (Qiagen) and cleaned using Amicon ultra 30K centrifugal filters (Sigma-Aldrich Merck, Israel). A DNA mix of amplified V gene segments was prepared at a ratio of 45% Vγ, 5% Vε, 25% Vκ, and 25% Vλ. Production of combinatorial light-heavy scFv libraries was performed by PCR reactions using the same reagent as the first PCR, but at 100 μl per reaction, with 100 ng of the V-gene mix, with “pull-through” primers (complementary to the overhangs flanking the restriction site of each product from the first PCR) at a concentration of 250 nM. Multiple recombination reactions (18-24) were prepared for ligation without primer and PCR was performed using the following program: 3 min at 98 °C, 5 cycles of 98 °C 20 sec+60 °C 60 sec+72 °C 60 sec. Primers were then added and the reaction was performed using the following program: 1 min at 98 °C, 30 cycles of 98 °C 20 sec+67 °C 60 sec+72 °C 45 sec and a final elongation stage of 72 °C for 3 min.

PCR products were concentrated by ethanol precipitation, ran on a 1% agarose gel, extracted using gel extraction kit (Qiagen) and cleaned using Amicon ultra 30K centrifugal filters (Sigma-Aldrich Merck). The pLibGD vector (described below) and the purified scFv DNA (at least 4 g vector and 2 m scFv) were restricted using hi-fidelity NcoI and NotI enzymes (NEB; MA, USA) according to the manufacturer's instructions. The vector was further treated by QuickCIP (NEB) according to the manufacturer's instructions. The restricted vector was cleaned by extraction from a 1% agarose gel and centrifugal filters as in previous steps. Restricted scFv were purified using PCR cleanup columns (Qiagen).

Ligation reactions of 20 μl were set up according to the manufacturer's instructions using 130 ng vector and 70 ng insert (producing a 3:1 ratio) and carried out at 10 °C overnight. A total of at least 3 μg DNA was ligated. Ligations were heat inactivated, cleaned by PCR cleanup columns, and concentrated by Amicon 30K centrifugal filters.

Ligated libraries were transformed to SS320 electrocompetent bacteria (Lucigen; WI, USA) according to manufacturer's instructions. Each library was divided into 2 transformations and seeded on three 15 cm 2YT-agar dishes containing 100 g/ml carbenicillin and 2% glucose. Dishes were incubated overnight at 30° c. Serial dilutions of transformations were seeded on separate kanamycin and ampicillin dishes to estimate transformation efficiencies. Libraries of >10⁷ were considered of sufficient quality and used further.

The next day, SS320 were scraped off of the dishes using 6 ml 2YT, diluted to O.D=0.1 in 60 ml 2YT supplemented with 100 μg/ml carbenicillin and 2% glucose, grown to O.D=0.5, and infected with KO7 helper phage (NEB) diluted 1:1000 for 30 minutes at 37 °C. The bacteria were then centrifuged at 3000 g for 10 minutes, resuspended in 200 ml 2YT+100 μg/ml carbenicillin+25 μg/ml kanamycin and grown at least overnight or up to 24 hours at 30 °C with 250 RPM shaking in baffled flasks to produce scFv-displaying phages.

The next day, bacteria were centrifuged for 10 minutes at 16,000 g. Supernatant was moved to fresh tubes and phages were precipitated by adding PEG/NaCl stock (PEG-8000 20%, NaCl 2.5 M) to a final concentration of 20% (1:4 ratio of PEG-NaCl stock to supernatant). Samples were incubated on ice for 20 minutes and centrifuged at 16,000 g, 4 °C for 30 minutes. Supernatant was discarded and the pellet was centrifuged again for 2 minutes to remove the remaining supernatant. Pellet was resuspended with 10 ml PBS/100 ml culture and centrifuged for 10 minutes at 16,000 g to remove residual bacteria cell debris. Samples were then subjected to a second identical round of PEG-NaCl precipitation and resuspended with 4 ml PBS/100 ml culture. Samples were centrifuged for 15 minutes at 20,000 g to remove residual debris and purified phages were supplemented with 50% glycerol and 2 mM EDTA and stored at −80 °C until use.

Screening of Phage Display Libraries for Allergen-Specific scFv

Isolation of allergen-specific scFv was done by panning phage libraries using either the natural purified allergen or recombinant allergen variants with modified suspected epitopes. Maxisorp high-binding 96-well plates (Nunc) were coated with 100 μl of 5 μg/ml allergen solution in PBS or with 2% BSA solution in PBS (8 wells per library). OmniMAX™ bacteria (Thermo Fisher Scientific; MA, USA) were seeded in 2YT+Tetracycline (5 ug/ml) and grown overnight at 37 °C with 250 RPM shaking.

The next day, OmniMAX™ bacteria were diluted in 2YT+Tetracycline to 0.1 O.D, grown to O.D=0.6-0.8 at 37 °C with 250 RPM shaking and kept on ice until use. Phage stock (2-4 ml) were defrosted, purified by PEG-NaCl purification (as above) and resuspended with 1 ml PBST (PBS+0.05% tween). A sample of un-panned phage stock was put aside for input measurement. If negative selection was performed, maxisorp plates were washed with 200 μl/well PBSTx3 and then phage solution was incubated in BSA-coated wells to remove non-specific binders at 100 μl/well for 1 hour at 4 °C with gentle shaking. Phage solutions were then moved to allergen-coated wells and incubated for 1 hour at 4 °C with gentle shaking. If no negative selection was performed, phage-PBST solution was added directly to allergen-coated wells. Plates were then washed twice with 200 μl/well PBST to remove unbound phages. Bound phages were eluted by incubation for 5 minutes with 100 μl/well of 100 mM HCl at R.T with gentle shaking. Elution reaction was stopped with 12.5 μl/well of Tris 1M, pH 11.

Eluted samples were added to 5 ml OmniMAX™ at required O.D and incubated for 30 minutes at 37 °C with 250 RPM shaking. Panning output titration was assessed by performing serial 10-fold dilutions with a sample of the infected stocks and seeding in triplicates 5 μl-drops on LB-agar dishes with carbenicillin or kanamycin or tetracycline. Remaining output was propagated by super-infection with 1:100 KO7 helper stock at 1:1000 for 45 minutes at 37 °C with 250 RPM shaking. Super-infected bacteria stocks were completed to 50 ml 2YT supplemented with carbenicillin and kanamycin and grown overnight at 37 °C with 250 RPM shaking to produce phages for the next round of panning. Panning input titration was assessed by performing serial 10-fold dilutions of input samples, infecting OmniMAX™ bacteria for 30 minutes at 37 °C with 250 RPM shaking and seeding triplicate drops on carbenicillin and kanamycin LB-agar dishes.

Subsequent panning rounds were performed by performing a single PEG-NaCl precipitation of the overnight output propagation and using it as input. From one panning round to the next, the number of wash cycles was increased, and the number of panning wells was decreased to increase panning stringency (3-to-4 panning cycles per library).

To isolate individual allergen-specific scFv, output serial dilutions of a chosen round were seeded onto LB-agar-carbanicillin dishes and grown overnight at 37 °C. The next day, individual colonies were inoculated into mini-tubes containing 300 μl 2YT+carbanicillin+1:1000 KO7 and grown overnight at 37 °C with 250 RPM shaking. The next day, supernatants from mini tubes were assayed by ELISA using plates coated with the allergen or BSA. The scFv from supernatants that bound specifically to the allergen and not to BSA were amplified by PCR with primers flanking the scFv region of the pLibGD plasmid. PCR products that were consistent with a full-length scFv were subjected to standard PCR cleaning by ExoI and rSAP restriction enzymes (NEB) and sequenced by standard sanger reactions (Hylabs). Full-length monoclones were cloned into mammalian expression plasmids (pSF) and expressed in HEK-293T cells as IgGs.

### Single Cell Sorting of Allergen Specific B Cells

Peanut allergy patients PBMC were thawed, washed with PBS, and stained for viability (LIVE/DEAD near-IR kit, Thermo-fisher) according to manufacturer's instructions. Cells were then incubated on ice for 1 hour with target allergens at varying concentrations according to allergen type. Allergens used were either natural purified allergens that were fluorescently labeled with alexa-fluor protein labeling kit (Thermo-fisher, a mix of allergens labeled with 2 different fluorophores, according to manufacturer's instructions), OR WT recombinant allergens with HA-tags on either C or N terminus, OR biotin-avidin labeled WT recombinant allergens (a mix of allergens labeled with 2 different fluorophores). Cells were then washed and stained with flourophore-conjugated antibodies for the following markers: CD14, CD16, IgM, IgD, CD3, CD19, IgG1. If using HA-tagged allergens, two anti-HA antibodies with different fluorophore conjugations were also added. Cells were then washed and sorted on an ARIA-III sorting flow cytometer. Single allergen-specific B cells (LIVE/DEADdim CD14-CD16-IgD-IgM-CD3-CD19+IgG1+allergen fluorophores double positive) were sorted into 96-well plates containing 4 μl/well ice-cold lysis buffer (PBS×0.5, 10 mM DTT, 8U RNAse inhibitor). Several wells were left empty in each plate as negative controls for PCR.

Isolation of Antibody Genes from Sorted Cells and Antibody Expression

Single sorted allergen-specific B cell lysates were directly subjected to reverse-transcription (SSIV, Invitrogen, according to manufacturer's instructions). Two sequential PCR reactions (2nd PCR nested) were performed to amplify heavy chain genes (Hotstart taq polymerase, NEB) and light chain genes (Kapa hot-start PCRF mix) using a mix of primers that cover the majority of known antibody gene alleles. PCR products were sequenced and aligned to the genome. Where a cell had reliable sequences for both heavy and light chains, sequences were cloned into mammalian expression plasmids (pSF) and expressed in HEK-293T cells as IgGs.

### Preparation of Yeast Surface Display Mutant Saturation Library and Flow-Cytometric Cell Sorting

A library consisting of Ara h 6 variants with single mutations in each residue was ordered from TWIST Bioscience (CA, USA) and cloned into a YSD vector which is similar to pCHA. To display the Ara h 6 library on the surface of the yeast denoted as S0, the library was grown in an SDCAA selective medium (2% dextrose, 0.67% Difco yeast nitrogen base, 0.5% Bacto casamino acids, 0.52% Na2HPO4, and 0.856% NaH2PO4·H2O) and induced for expression with a galactose medium (as for SDCAA, but with galactose 2%, instead of dextrose) according to an established protocol (Chao, G., Lau, W., Hackel, B. et al. Isolating and engineering human antibodies using yeast surface display. Nat Protoc 1, 755-768 (2006)). Ara h 6 expression was detected by an anti-Myc antibody conjugated to FITC (Miltenyi Biotec, Bergisch Gladbach, Germany) and anti-Ara h 6 IgG binding was detected by secondary affinipure donkey anti-human IgG (H+L) antibody conjugated with APC (Jackson ImmunoResearch Laboratories, PA, USA,). For pairwise selectivity screen, ~1×10⁶ yeast cells were incubated with different anti-Ara h 6 IgG in a binding buffer (100 mM Tris, pH=8.0, 1 mM CaCl2), 1% BSA) for 1 h at room temperature. Then, the cells were washed with the binding buffer and incubated for 30 min with anti-Myc-FITC and anti-human IgG-APC antibodies. Then, the cells were washed again with a binding buffer and sorted for the low-selective variants by conducting several independent sorts, using S3E cell sorter (Bio-Rad). Ara h 6 variants that showed a low binding affinity toward the anti-Ara h 6 IgG, i.e., top the lowest up to 3% of the entire population, were selected.

### High-Throughput Sequencing Library Preparation

A YSD vector containing the Ara h 6 gene was isolated from the naïve library and from the sorted libraries by using Zymoprep Yeast Plasmid Miniprep II (Zymo research, Irvine, CA) according to the manufacturer's protocol. Using this kit, ~200 ng of DNA was isolated from each yeast library. The extracted vectors were sent to the NGS laboratory of Hy Laboratories (Hylabs, Rehovot, Israel) for a first and secondary PCR of twenty and eight cycles (respectively), using the Fluidigm Access Array primers, to add the adaptors and barcodes. Then, the DNA library samples were purified with AmpureXP beads (Beckman Coulter, Brea, CA) and the concentrations of the samples were determined in a Qubit by using the DNA high sensitivity assay. The samples were pooled and then ran on a TapeStation (Agilent, Santa Clara, CA) to verify the size of the PCR product. As a final quality test, the pools were subjected to qRT-PCR to determine the concentration of the DNA that can be sequenced. The pools were then loaded for sequencing on an Illumina Miseq, using the 600v2 kit.

### Deep Sequencing Reads Analysis

Paired-end reads were analyzed and filtered for quality using the fastp command-line preprocessing tool (Chen, S., Zhou, Y., Chen, Y., & Gu, J. (2018). fastp: an ultra-fast all-in-one FASTQ preprocessor. Bioinformatics (Oxford, England), 34(17), i884-i890.). All sequences where over 10% or 20% of the sequence had a Phred quality score under 20, depending on whole library quality, were discarded from subsequent analysis. Reads were then aligned based on a probabilistic model of their overlapping region, implemented within the pandaseq assembler (Masella, A. P., Bartram, A. K., Truszkowski, J. M. et al. PANDAseq: paired-end assembler for illumina sequences. BMC Bioinformatics 13, 31 (2012).). Translated sequences were filtered for the appearance of expected mutations (single mutation per sequence, i.e., single mutation per variant) and analyzed for sequence enrichment:

$$Enrich X i = ( \frac{f {S}_{1} a {a}_{i}}{f {S}_{0} a {a}_{i}} )$$

Where aaᵢ is a specific amino acid at position i, fS₁ is the fraction of reads of the given amino acid at position i in the sorted library and fS₀ is the same fraction, in the input library. This calculation provides the enrichment of each specific Ara h 6-point mutant.

For convenience, the following can also be used to denote the increased index of a specific amino acid at position i.

$$IN {aa}_{i} = \frac{f {S}_{1} a {a}_{i}}{f {S}_{0} {aa}_{i}}$$

integration of this information over all mutations at a given position is performed by calculating the Shannon entropy of each position:

$${ENT}_{i} = {{\sum }_{z = 1}}^{20} \frac{{INaa}_{z}}{{{\sum }_{j = 1}}^{20} {INaa}_{j}} ln {{\sum }_{z = 1}}^{20} \frac{{INaa}_{z}}{{INaa}_{j}}$$

Where i is a given position, INaa_{z} represents the increase index for a given amino acid, normalized by the increase index of all amino acids.

### Ara h 6 Purification

Ara h 6 variant purification, Ara h 6 WT (SEQ ID NO: 2) and mutants were cloned into pET28 plasmid. Ara h 6 was fused to DNA encoding His-tagged at the N terminus (Ara h 6-His*6). All variants were expressed under the transcriptional control of the T7 promoter. Cells were grown at 37 °C until an OD of 0.5-0.8 was reached, induction was carried out for 3 h by addition of 0.5 mM IPTG at 37 °C. Cells were harvested (4800 g for 30 min) and cells pellet was resuspended with lysis buffer (50 mM Tris pH 8.0, 350 mM NaCl, 10% v/v glycerol, 0.2% Triton X-100, 250 U Benzonase, 0.2 mM PMSF and 1 mg/ml Lysozyme), lysis was done by sonication (35% amplitude, 10 sec on and 30 sec off for 2 min). Lysates were centrifuged (15000 g, 45 min) and supernatant was loaded on pre-washed with binding buffer (50 mM Tris pH 8.0, 350 mM NaCl and 10% v/v glycerol) Ni-NTA beads and incubated at 4 °C for 1 hr. The beads were washed with a binding buffer containing increased imidazole concentration. Then, Ara h 6 purity was improved, and imidazole concentration was diluted by size-exclusion chromatography (SEC). The fractions containing Ara h 6 were collected and concentrated by 3 kDa centricones (Amicon, Mercury), protein concentration was measured by the absorbance at 220 nm.

### Analysis of Binding to Monoclonal Antibodies by ELISA

The concentrations of Anti-Ara h 6 IgG required to give 50% of maximal binding to WT-Ara h 6 and Ara h 6 variants (EC50) were determined using an ELISA. Briefly, wells of 96-well microtiter plates (Thermo Fisher Scientific, Waltham, MA) were coated overnight at 4 °C with 200 ng of Ara h 6. Plates were blocked with 0.5% BSA in PBS (200 l/well) for 1 hour at room temperature. Anti-Ara h 6 IgGs were prepared by a serial dilution in PBS, added to the Ara h 6-coated wells and incubated for 1 hr at room temperature. Following washing steps, the amount of bound IgG was detected by incubation with the Goat-anti-human IgG conjugated with HRP polyclonal antibody (Jackson ImmunoResearch Laboratories, PA, USA) and then TMB substrate.

All incubation steps were performed in PBS containing 0.5% BSA and 0.05% Tween 20. The highest concentrations of Anti-Ara h 6 IgG are saturating, and the amount bound to Ara h 6 reaches a maximum at these levels.

Computational Design of Variants with Mutations at Multiple Sites

Based on experimental results which identified point mutations that reduce binding to mAbs and/or to patient plasma, computational protein design tools are used to generate variants with combinations of mutations that are predicted to maintain their stability. The NMR structure of Ara h 6 is energetically optimized (for each of the NMR states). Next, a combinatorial mutagenesis scanning tool is used to perform Monte-Carlo sampling of up to 5 simultaneous mutations, in cases where mutations are combined at the epitope level, or up to 25 simultaneous mutations, in cases where mutations are combined at the protein level. This allows minimization of the backbone upon side chain mutagenesis, generating up to 250 structures. Mutations are evaluated by the computed AG, the change in the free energy of the protein upon mutation. Sequences are ranked by their AG, eliminating any structure with a significant increase in AG and by their sequence diversity, to eliminate experimental testing of near identical protein sequences.

### RBL SX-38 Cell Degranulation Assays

RBL SX-38 cells were received from Prof. Stephen Dreskin in UC Denver, with permission from BIDMC in Boston. Cells were cultured at 37 °C, 5% CO₂ in maintenance media containing 80% MEM, 20% RPMI 1640, 5% FCS (not heat-inactivated), supplemented with L-glutamin, Penicillin-Streptomycin and G418 at 1 mg/ml (all from Gibco-Thermo fisher, USA). At least 48 hours before assay, cells were split and expanded in assay media (maintenance media without RPMI and G418). On day of assay, cells were detached using 0.05% Trypsin-EDTA (Gibco), centrifuged at 300 g for 10 minutes, and resuspended in assay media supplemented with 5-10% clinical sample (plasma/serum from peanut allergy patients, dilution varied from sample to sample) to a final concentration of 2.5×10⁶ cells/ml. If plasma was produced with any anticoagulant other than heparin, the sample was first supplemented with 30 U/ml Heparin (Sodium-Heparin, Sigma) and incubated at room temperature for 10 minutes before adding to cells. Cells were then seeded at 50 μl per well (final 125,000 cells/well) in 96-well flat-bottom tissue culture plates (Greiner bio-one, Austria) and cultured overnight. The next day, activation solutions were prepared by diluting allergens or un-related protein negative controls at varying concentrations in Tyrode's buffer (137 mM NaCl, 2.7 mM KCl, 0.4 mM NaH₂PO₄, 0.5 mM MgCl₂, 1.4 mM CaCl₂, 10 mM Hepes pH 7.3, 5.6 mM glucose, 0.1% BSA, pH adjusted to 7.4, prepared in a water composition of 80% ddw and 20% D20 heavy water, Merck-Sigma Aldrich, Israel). Cells were then washed 3 times with Tyrode's buffer prepared with ddw only, and 100 μl allergen activating solution was added to appropriate wells in duplicates. For each allergen, 5-6 concentrations at 10-fold dilutions were used. Each clinical sample was tested for WT allergen, variant allergens, and an unrelated protein as negative control (KLH, Sigma). Duplicate wells were also prepared with a lysis buffer (Tyrode's buffer with 1% Triton x-100, Fisher Scientific) for measuring total degranulation and with Tyrode's buffer alone for measuring background degranulation. Cells were then incubated for 1 hour at 37 °C, 5% CO₂. Immediately after incubation, 30 μl of each well were transferred to a corresponding well in a clear non-binding 96-well plate (Greiner Bio-one) and supplemented with 50 μl PNAG colorimetric substrate (4-Nitrophenyl N-acetyl-β-D-glucosaminide prepared in 0.1M citric acid to final concentration 1.368 mg/ml pH4.5). Reactions were incubated for 1 hour at 37 °C with gentle shaking in the dark and then 100 μl stop solution (0.2M glycine at pH 10.7) was added to halt reaction and develop color. Optical densities were read at 405 nm for signal and at 630 nm for background absorbance using the Synergy LX microplate spectrophotometer reader (Biotek, Vermont). After subtraction of background absorbance, net degranulation was calculated by dividing the OD of each cell by the OD in the corresponding lysis buffer wells (total degranulation) and subtracting the OD of buffer only wells (background degranulation).

BAT Assays Fresh whole blood samples in heparinized tubes (BD biosciences) were divided into 100 ul per tube. Allergens and controls were diluted in RPMI1640 (Biological Industries) to ×2 stocks, added 1:1 to tubes (final volume 200 ul) and incubated for 30 minutes in a 37 °C, 5% CO₂ humidified incubator. The dose range used was 0.1-10000 ng/ml. Crude peanut extract (CPE), fMLP and anti-human IgE antibodies were used as positive controls. KLH protein was used as a negative control. The reaction was stopped by incubation on ice for 5 min. A cocktail of fluorophore-conjugated antibodies was added directly to the samples to detect the following markers: CD203c, CD63, HLA-DR, CD45, CD123. Cells are incubated for 30 min on ice. RBC lysis was performed with a kit according to manufacturer's instructions (BD FACS lysing solution), and cells were washed and analyzed by flow cytometry. Cells were gated for basophil detection and activation rate (% CD63-positive basophils) was measured. At least 500 basophils were analyzed per tube.

### T Cell Activation Assay

PBMC were isolated from heparinized peanut allergy patient blood samples. Cells were washed with PBS, stained with Celltrace violet (Thermo-fisher) according to the manufacturer's instructions, and seeded in 96-well round bottom plates at 0.2-0.5×106 cells/well (according to available number of cells following purification and staining) in X-vivo15 media supplemented with 5% human AB serum (Biotag) and 1% penicillin-streptomycin solution (Biological industries). Recombinant WT and variant allergens were purified by Rapid Endotoxin Removal Kit (Abcam), tested for residual endotoxin contamination (LAL Chromogenic Endotoxin Quantitation Kit, Pierce), diluted in same media as cells, sterilized by 0.22 μM filtration and added to cells to a final concentration of 50 μg/ml in 200 μl per well. Inactivated wells (baseline, media only) and each allergen were tested per patient by 3 or more replicate wells. Each assay included healthy donor samples alongside patients as negative controls for assay quality assurance. Final endotoxin levels in wells for all allergens were <0.5 EU. Cells were incubated for 7 days in a 37 °C, 5% CO₂ humidified incubator. If media in any of the wells changed to yellow during the incubation period, half of the media was replaced with fresh media for all wells. After 7 days, cells were harvested, stained for viability (LIVE/DEAD stain, Thermo-fisher), stained with anti-CD3 and anti-CD4 fluorophore-conjugated antibodies (Biolegend; USA) and analyzed by flow cytometry. Live T helper cells were gated (LIVE/DEADlowCD4+CD3+) and the percent of proliferating cells (Celltracedim/Total T helper) was measured. A positive result (allergen causes activation of patient T cells) was determined where the mean of allergen-stimulated wells was greater than mean+3×SD of unstimulated wells.

### CD of Ara h 6 WT and Variants

Circular dichroism (CD) spectroscopy is a useful technique for analyzing purified protein secondary structure and folding properties in solution using very small amounts of protein. It is based on the differential absorbance of left- and right-hand circularly polarized light by a chromophore. The CD analysis of proteins is based on the amide chromophore in the far UV region (below 260 nm). For example, α-helical proteins have negative bands at 222 nm and 208 nm and a positive band at 193 nm, whereas proteins with well-defined antiparallel β-pleated sheets (β-sheet) have negative bands at 218 nm and positive bands at 195 nm. The circular dichroism spectra of the recombinant Ara h 6 proteins were measured on Chirascan CD spectrometer (Applied Photophysics) at Bar Ilan university. Far-UV CD spectra from 200-260 nm were acquired with a 10 mm path-length cuvette. The purified Ara h 6 recombinant WT and D12 variant were measured in a PBS buffer and concentrations were determined using SEC-HPLC compared to a reference natural protein. Spectra were acquired at 25 °C and at a range of temperatures from 20-90 °C to assess the stability of the proteins.

### Strains, Plasmid and Growth Conditions

Escherichia coli subcloning efficiency DH5alpha competent cells (Invitrogen) were routinely used for all cloning procedures, Escherichia coli OmniMAX™ (Thermo Fisher scientific) were used for phage display libraries screening, Escherichia coli BL21 (DE3) cells were used for Ara h 6 purification. All strains were grown on 2YT broth and LB agar plates at 37 °C. A phagemid was used for scFvs phage display libraries derived from peanut allergic patients. tPCR was used to insert a non-specific scFv that was derived from a healthy donor and designed with a non-structured GGGSx4 linker and to add restriction sites at either ends of the scFv segment—NcoI at the 5′ end and NotI at the 3′ end (the modified plasmid was marked internally as pLibGD). Plasmid pET28 (Invitrogen) was used for recombinant purification of Ara h 6 and mutants. Transformations for scFv display were performed using SS320 electrocompetent Escherichia coli (Lucigen).

### Example 2: Epitope Mapping and De-Epitoping of Ara h 6 Polypeptides

Objective: The overall objective is to develop a basis for defined targeted mutation of allergenic polypeptides that are stable, retain their T cell activation activity, but have reduced binding to IgE antibodies. For the purpose of immunotherapy, the functionality of these Ara h 6 variant polypeptides includes maintaining immunogenicity, e.g., by the ability to activate T-cells. This series of experiments was performed to identify and map conformational and linear epitopes on the peanut allergen Ara h 6, based on the binding of either patients' sera or specific monoclonal antibodies isolated from peanut allergic patient samples; and to identify amino acid residues within the Ara h 6 mAb binding epitopes that contribute to binding, and which when mutated are not predicted to destabilize the protein.

### Results

The pipeline for single epitope mapping and de-epitoping of the peanut allergens Ara h 6 included two stages—(1) discovery of Ara h 6-specific antibodies (i.e., sera or isolated mAb) from peanut allergic patient samples that exhibit specific IgE binding to Ara h 6, as measured by ELISA assay and peptide array, and (2) mapping of the epitope that each antibody binds. Stage 1, mAb discovery, was carried out using scFv phage display libraries by amplification of the variable genes and construction of scFv that are fused to pIII protein and displayed on phages, or by Ara h 6 specific B cells single cell sorting, followed by sequencing of the variable region and production of recombinant mAbs.

Briefly, scFv phage display library from PBMC of one peanut allergic patient were generated as described in Example 1 followed by a panning process of these libraries, identifying three Ara h 6 specific mAbs. Single sorted allergen-specific B cell lysates of 11 peanut allergic patients were generated as described in Example 1, identifying 15 Ara h 6 specific mAbs. All 18 mAbs were cloned into mammalian expression plasmids (pSF) and expressed in HEK-293T cells as IgGs. The epitope mapping procedure, as described below, was completed for 14 Ara h 6 IgG mAbs.

In the second stage, the anti-Ara h 6 specific purified mAbs were used for epitope mapping in two complementary approaches:

Approach A. Screening of site saturation variant library using yeast surface display (YSD) (Siloto and Weselake (2012) Site saturation mutagenesis: Methods and applications in protein engineering. Biocatalysis and Agricultural Biotechnology, Volume 1(3):181-189) (Cherf G M, Cochran J R. (2015) Applications of Yeast Surface Display for Protein Engineering. Methods Mol Biol. 1319:155-75.)

Epitope mapping using Ara h 6 YSD saturation library: For the purpose of epitope mapping, a two-step procedure was performed. First, the Ara h 6 point mutants library was sorted for expression only, collecting those variants that undergo successful YSD, resulting in a sorted library that will be referred to as S1. The threshold for expression was defined as the fluorescence value that is higher than the unstained cells (background). Each cell that had higher fluorescent signal than the background was collected (S1 lib). Next, S1 library binding to 14 mAbs was assessed. Yeast cells that displayed Ara h 6 variants and exhibited mAb binding signal (APC) in the lower 3% of the population were sorted.

Deep sequencing was performed to each mAb in order to identify the positions that affect binding to the specific mAb. As the library has undergone a selection for expression and lower mAb binding, sequencing results were analyzed by means of enrichment calculations. Each unique DNA sequence that encodes a point mutant was counted and the fold change in its relative abundance was calculated, to serve as an indirect estimate for the change in mAb binding.

At least two (2) conformational epitopes were identified in Ara h 6.

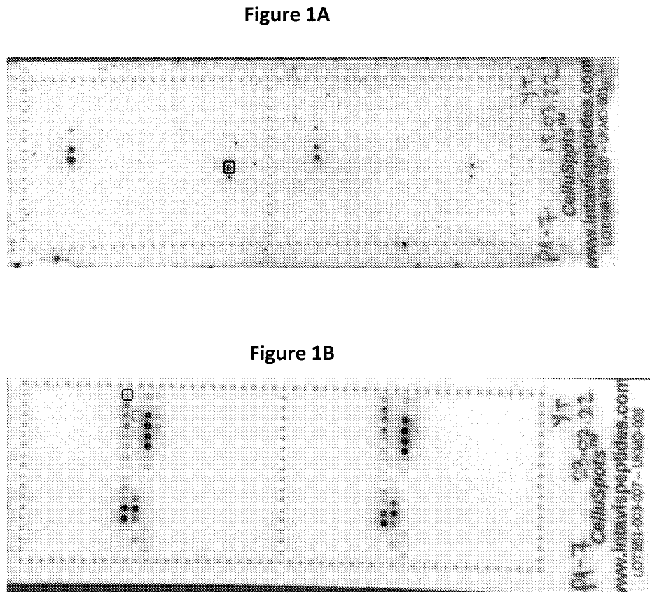

Approach B. Peptide microarray assay was performed as described in Example 1 with purified mAbs (commercial IgE or IgG) to map some of the consecutive epitopes on the allergen Ara h 6. This method was also used to validate the data from the YSD saturation for linear epitopes. In Ara h 6, five linear epitopes were identified, confirmed with 6 mAbs that were analyzed with the peptide array. Next, mAbs mapped to Ara h 6 were assayed with the mutated “de-epitoping” spots containing array to screen for those peptides showing the most significant decrease in binding. The results of representative arrays are shown for the linear epitope mapping (FIG. 1A) and de-epitoping (FIG. 1B) of Ara h 6 mAb.

IgE epitope mapping and de-epitoping of Ara h 6 based on plasma from allergic patients (See Example 3). Critical positions in 3 epitopes were identified using peptide microarray similar to the process in Approach B. However, instead of mapping isolated monoclonal antibodies, the IgE repertoire from allergic patient plasma was used as described in Example 3.

### Summary

Table 1 summarizes embodiments of the Ara h 6 variants with mutations at positions with respect to WT Ara h 6, amino acid mutations, and epitopes thereof. The mutation details presented in Table 1 were collated from the results of Example 2 and Example 3.

TABLE 1
Ara h 6 Variants*

| | Variant position | | | |
|---|---|---|---|---|
| | numbered | Uniprot | | |
| WT Ara h 6 | according to | numbering | | Part of |
| SEQ ID NO: 2 | SEQ ID NO: 2 | (A5Z1R0) | Mutations | epitope(s) |
| M | 1 | 22 | | |
| R | 2 | 23 | S | L1 |
| R | 3 | 24 | D, S | L1 |
| E | 4 | 25 | | L1 |
| R | 5 | 26 | D | L1 |
| G | 6 | 27 | | L1 |
| R | 7 | 28 | D | L1 |
| Q | 8 | 29 | A, S | L1 |
| G | 9 | 30 | | L1 |
| D | 10 | 31 | A, S, K | L1 |
| S | 11 | 32 | | L1 |
| S | 12 | 33 | R, D, N | L1 |
| S | 13 | 34 | | L1 |
| C | 14 | 35 | | L1 |
| E | 15 | 36 | R | |
| R | 16 | 37 | S, D | C1 |
| Q | 17 | 38 | R | |
| V | 18 | 39 | | |
| D | 19 | 40 | Q, L, R | C1, C2 |
| R | 20 | 41 | D | |
| V | 21 | 42 | | |
| N | 22 | 43 | F | C2 |
| L | 23 | 44 | | |
| K | 24 | 45 | D | C2 |
| P | 25 | 46 | | |
| C | 26 | 47 | | |
| E | 27 | 48 | | |
| Q | 28 | 49 | S | |
| H | 29 | 50 | | |
| I | 30 | 51 | | |
| M | 31 | 52 | | |
| Q | 32 | 53 | | |
| R | 33 | 54 | Q, K | C3 |
| I | 34 | 55 | | |
| M | 35 | 56 | A | |
| G | 36 | 57 | | L2 |
| E | 37 | 58 | A, T, S | L2 |
| Q | 38 | 59 | A, S | L2 |
| E | 39 | 60 | | L2 |
| Q | 40 | 61 | S | L2 |
| Y | 41 | 62 | D | L2 |
| D | 42 | 63 | K, E, G | L2 |
| S | 43 | 64 | | L2 |
| Y | 44 | 65 | | L2 |
| D | 45 | 66 | A, Q | L2 |
| I | 46 | 67 | S, G, R | L2 |
| R | 47 | 68 | S | L2 |
| S | 48 | 69 | | L2 |
| T | 49 | 70 | | L2 |
| R | 50 | 71 | | L2 |
| S | 51 | 72 | | L2 |
| S | 52 | 73 | | L2 |
| D | 53 | 74 | | L2 |
| Q | 54 | 75 | | L2 |
| Q | 55 | 76 | | |
| Q | 56 | 77 | | |
| R | 57 | 78 | D | |
| C | 58 | 79 | | |
| C | 59 | 80 | Y | |
| D | 60 | 81 | | |
| E | 61 | 82 | F | |
| L | 62 | 83 | | |
| N | 63 | 84 | | C4 |
| E | 64 | 85 | S | |
| M | 65 | 86 | | |
| E | 66 | 87 | | |
| N | 67 | 88 | | |
| T | 68 | 89 | | |
| Q | 69 | 90 | | |
| R | 70 | 91 | | |
| C | 71 | 92 | | |
| M | 72 | 93 | | |
| C | 73 | 94 | | |
| E | 74 | 95 | R | C1 |
| A | 75 | 96 | | |
| L | 76 | 97 | | L3 |
| Q | 77 | 98 | | L3 |
| Q | 78 | 99 | L | L3 |
| I | 79 | 100 | | L3 |
| M | 80 | 101 | | L3 |
| E | 81 | 102 | A, R | L3, C1 |
| N | 82 | 103 | T | L3 |
| Q | 83 | 104 | N, K | L3 |
| C | 84 | 105 | | L3 |
| D | 85 | 106 | | L3 |
| R | 86 | 107 | S, D | L3 |
| L | 87 | 108 | | L3 |
| Q | 88 | 109 | | L3 |
| D | 89 | 110 | N, R, G | L3 |
| R | 90 | 111 | D | L3 |
| Q | 91 | 112 | A, S | |
| M | 92 | 113 | | L4 |
| V | 93 | 114 | | L4 |
| Q | 94 | 115 | | L4 |
| Q | 95 | 116 | | L4 |
| F | 96 | 117 | | L4 |
| K | 97 | 118 | I | L4 |
| R | 98 | 119 | D, L | L4 |
| E | 99 | 120 | M | L4 |
| L | 100 | 121 | | L4 |
| M | 101 | 122 | | L4 |
| N | 102 | 123 | | L4 |
| L | 103 | 124 | | |
| P | 104 | 125 | | L5 |
| Q | 105 | 126 | | L5 |
| Q | 106 | 127 | K, H | L5, C3 |
| C | 107 | 128 | | L5 |
| N | 108 | 129 | P, E, D | L5, C4 |
| F | 109 | 130 | | L5, C4 |
| R | 110 | 131 | E, S | L5 |
| A | 111 | 132 | | L5 |
| P | 112 | 133 | | L5 |
| Q | 113 | 134 | D, I | L5 |
| R | 114 | 135 | D, H, A, G | L5, C5 |
| C | 115 | 136 | | L5 |
| D | 116 | 137 | K, M | L5, C5 |
| L | 117 | 138 | | L5 |
| D | 118 | 139 | R, T | L5 |
| V | 119 | 140 | | |
| S | 120 | 141 | | |
| G | 121 | 142 | | |
| G | 122 | 143 | | |
| R | 123 | 144 | D | |
| C | 124 | 145 | | |

*Highlighted positions refer to shared substitutions appearing in Ara h 6 variants D119, D154, D158, D160, D179 (SEQ ID NOs: 46, 76, 80, 82, and 101, respectively).

### Example 3: IgE Epitope Mapping and De-Epitoping Based on Allergic Patients' Plasma Samples

Objective: Following through with the overall objective of developing a basis for defined targeted mutation of allergenic polypeptides that are stable, retain their functional characteristics, but have reduced binding to IgE allergenic antibodies, the objective of these experiments was to identify the consecutive linear IgE epitopes for peanut patients' plasma and analyze mutant variants thereof.

### Results

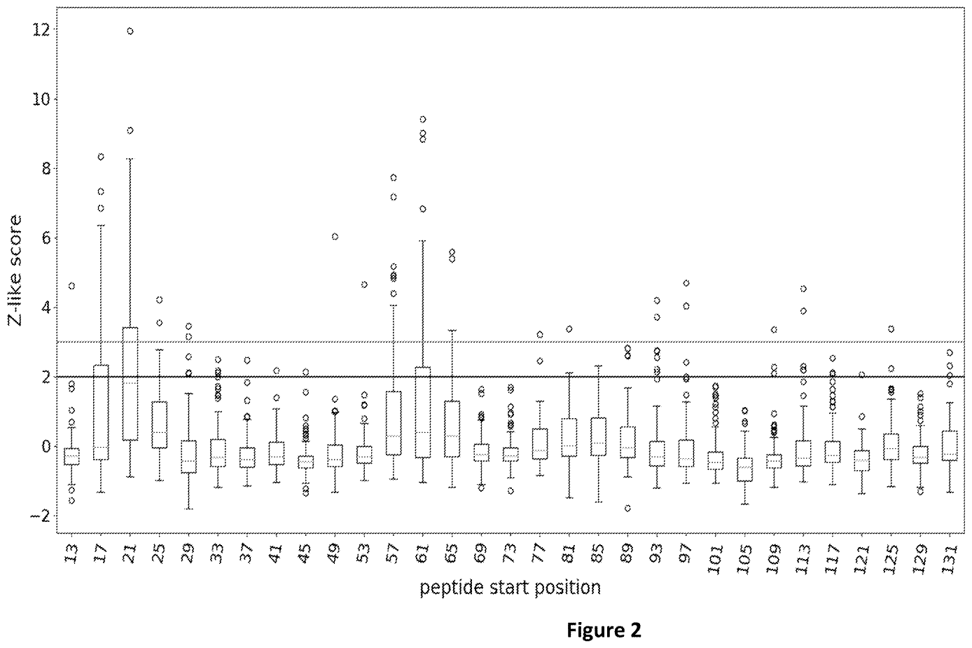

The same peptide arrays as in the purified mAbs analysis procedure were used to identify all linear epitopes on the allergen Ara h 6 of polyclonal IgE from allergic patient plasma. These arrays were assayed with the plasma of 216 peanut allergic patients, testing for plasma-derived IgE binding of Ara h 6-derived peptides. Of the tested plasma, 80 slides identified IgE binding to at least one peptide from Ara h 6. Analysis and clustering of peptide array results allowed for the mapping of all linear epitopes of the proteins (FIG. 2).

Based on the mapped epitopes, an additional array was synthesized, where for each Ara h 6 mapped epitope, the WT peptide was spotted along mutated peptides that were computationally designed to diminish IgE binding. Peptides were 15 amino acids long and included either point mutations or double substitution mutations. Next, plasma mapped to Ara h 6 were assayed with the mutated “de-epitoping” spots containing array to screen for those peptides showing the most significant decrease in binding (data not shown). Furthermore, the mutation/epitope details presented in Table 1 of Example 2 were collated from the results of both Example 2 and Example 3.

Two main linear epitope regions of IgE binding were mapped by peptide microarray using plasma of 80 allergic patients. Ara h 6 linear epitopes at the population level are calculated for each peptide by the relative deviation from slide median intensity (Z-like score). The distribution of all scores from all slides is plotted and shown as a box plot, where the x-axis corresponds to all overlapping peptides and the y-axis shows the distribution of Z-like scores. Black and gray lines signify 2 and 3 standard deviations, respectively, from the slide median intensities (FIG. 2).

### Example 4: Mutation of Single or Multiple Epitopes

Objective: Using the data collected in Examples 2 and 3, variants were designed with combinations of mutations.

Results: Mutations were combined based on computational prediction of the energetic effect of the mutations on protein stability. Calculations were performed starting from the solved structures of Ara h 6 (PDB accession 1W2Q). Several epitopes could be mutated within a single variant. Mutations included 1-7 substitution mutations within the epitope. The designed variants were produced in E. coli and tested to verify a reduction in binding to anti Ara h 6 mAbs by indirect Enzyme-Linked ImmunoSorbent Assay (ELISA).

TABLE 2
Amino Acid Sequences of Ara h 6 Variants

| SEQ | | |
|---|---|---|
| ID | | |
| NO: | Name (PLP) | SEQUENCE |
| 1 | A5Z1R0 | MAKSTILVALLALVLVAHASAMRRERGRQGDSSSCERQVDRVNLKPCEQHIM |
| | (natural) | QRIMGEQEQYDSYDIRSTRSSDQQQRCCDELNEMENTQRCMCEALQQIME |
| | | NQCDRLQDRQMVQQFKRELMNLPQQCNFRAPQRCDLDVSGGRC |
| 2 | Ara h 6 WT | MRRERGRQGDSSSCERQVDRVNLKPCEQHIMQRIMGEQEQYDSYDIRSTRS |
| | | SDQQQRCCDELNEMENTQRCMCEALQQIMENQCDRLQDRQMVQQFKREL |
| | | MNLPQQCNFRAPQRCDLDVSGGRC |
| 3 | Arah6_D6 | RDEDGDAGASRSCERQVDRVNLDPCESHIMQRIMGAAEQYKSYAIRSTRSSD |
| | | QQQRCCDELNEMENTQRCMCEALQQIMENQCDRLQNDAMVQQFKRELM |
| | | NLPQQCPFRAPQDCKLRVSGGRC |
| 4 | Arah6_D7 | RDEDGDSGSSDSCERQVDRVNLDPCESHIMQRIMGTSEQYESYQIRSTRSSD |
| | | QQQRCCDELNEMENTQRCMCEALQQIMENQCDRLQNDSMVQQFKRELM |
| | | NLPQQCEFRAPQHCKLTVSGGRC |
| 5 | Arah6_D8 | RRERGRQGDSNSCRRRVDRVFLDPCESHIMQRIMGEQEQYDSYDIRSTRSSD |
| | | QQQDCYDFLNSMENTQRCMCEALQLIMATNCDRLQRRQMVQQFIDMLM |
| | | NLPQQCNFRAPQACMLDVSGGRC |
| 6 | Arah6_D9 | RDEDGRSGDSSSCERQVDRVNLKPCEQHIMQRIMGEQEQYDSYAIRSTRSSD |
| | | QQQDCYDFLNSMENTQRCMCEALQLIMANKCDRLQRRQMVQQFIDMLM |
| | | NLPQQCNFRAPQACMLDVSGGRC |
| 7 | Arah6_D10 | RDEDGDSGKSSSCERQVDRVNLKPCEQHIMQRIMGEQEQYGSYASRSTRSSD |
| | | QQQRCCDELNEMENTQRCMCEALQQIMENQCDRLQGDQMVQQFKRELM |
| | | NLPQQCDFEAPDDCKLRVSGGRC |
| 8 | Arah6_D11 | SSEDGRSGDSSSCESQVDDVNLKPCEQHIMQRIMGSQESYDSYDGSSTRSSD |
| | | QQQRCCDELNEMENTQRCMCEALQQIMENQCDSLQDDQMVQQFKDELM |
| | | NLPQQCNFSAPIGCDLDVSGGDC |
| 9 | Arah6_D12 | RDEDGRAGDSSSCERQVDRVNLDPCESHIMQRIAGAAEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 10 | Arah6_D13 | RDEDGRAGDSSSCERQVDRVNLDPCESHIMQRIAGAAEQYDSYARRSTRSSD |
| | | QQQDCYDFLNSMENTQRCMCEALQLIMATNCDRLQRDQMVQQFIDMLM |
| | | NLPQQCDFEAPQACMLRVSGGRC |
| 11 | Arah6_D71 | RDEDGRAGDSSSCEDQVQRVNLDPCESHIMQRIAGAAEQDDSYARRSTRSS |
| | | DQQQRCCDELNEMENTQRCMCRALQQIMRNQCDRLQGDQMVQQFKLEL |
| | | MNLPQQCDFEAPQDCKLRVSGGRC |
| 12 | Arah6_D72 | RDEDGRAGDSSSCEDQVLRVNLDPCESHIMQRIAGAAEQDDSYARRSTRSSD |
| | | QQQRCCDELNEMENTQRCMCRALQQIMRNQCDRLQGDQMVQQFKLELM |
| | | NLPQQCDFEAPQDCKLRVSGGRC |
| 13 | Arah6_D73 | RDEDGRAGDSSSCEDQVQRVNLDPCESHIMQRIAGAAEQDDSYDRRSTRSS |
| | | DQQQRCCDELNEMENTQRCMCRALQQIMRNQCDRLQGDQMVQQFKLEL |
| | | MNLPQQCDFEAPQDCKLDVSGGRC |
| 14 | Arah6_D74 | RDEDGRAGDSSSCEDQVQRVNLKPCEQHIMQRIMGEQEQDDSYARRSTRSS |
| | | DQQQRCCDELNEMENTQRCMCRALQQIMRNQCDRLQGDQMVQQFKLEL |
| | | MNLPQQCDFEAPQDCKLRVSGGRC |
| 15 | Arah6_D75 | RDEDGRAGDSSSCEDQVQRVNLDPCESHIMQRIAGAAEQDDSYDRRSTRSS |
| | | DQQQRCCDELNEMENTQRCMCEALQQIMENQCDRLQGDQMVQQFKLEL |
| | | MNLPQQCDFEAPQDCKLDVSGGRC |
| 16 | Arah6_D76 | RDEDGRAGDSSSCEDQVQRVNLKPCEQHIMQRIMGEQEQDDSYDRRSTRSS |
| | | DQQQRCCDELNEMENTQRCMCEALQQIMENQCDRLQGDQMVQQFKLEL |
| | | MNLPQQCDFEAPQDCKLDVSGGRC |
| 17 | Arah6_D77 | RDEDGRAGDSSSCEDQVQRVNLKPCEQHIMQRIMGEQEQDDSYARRSTRSS |
| | | DQQQRCCDELNEMENTQRCMCEALQQIMENQCDRLQGDQMVQQFKLEL |
| | | MNLPQQCDFEAPQDCKLRVSGGRC |
| 18 | Arah6_D78 | RDEDGRAGDSSSCEDQVLRVNLKPCEQHIMQRIMGEQEQDDSYARRSTRSS |
| | | DQQQRCCDELNEMENTQRCMCRALQQIMRNQCDRLQGDQMVQQFKLEL |
| | | MNLPQQCDFEAPQDCKLRVSGGRC |
| 19 | Arah6_D79 | RDEDGRAGDSSSCEDQVLRVNLKPCEQHIMQRIMGEQEQDDSYARRSTRSS |
| | | DQQQRCCDELNEMENTQRCMCEALQQIMENQCDRLQGDQMVQQFKLEL |
| | | MNLPQQCDFEAPQDCKLRVSGGRC |
| 20 | Arah6_D80 | RDEDGRAGDSSSCEDQVLRVNLKPCEQHIMQRIMGEQEQDDSYDRRSTRSS |
| | | DQQQRCCDELNEMENTQRCMCEALQQIMENQCDRLQGDQMVQQFKLEL |
| | | MNLPQQCDFEAPQDCKLDVSGGRC |
| 21 | Arah6_D81 | RDEDGRAGDSSSCEDQVDRVNLKPCEQHIMQRIMGEQEQDDSYARRSTRSS |
| | | DQQQRCCDELNEMENTQRCMCEALQQIMENQCDRLQGDQMVQQFKLEL |
| | | MNLPQQCDFEAPQDCKLRVSGGRC |
| 22 | Ara h 6-e | MXXEXGXXGXSXSCXXXVXXVXLXPCEXHIMQRIXGXXEXXXSYXXXSTRSSD |
| | variant | QQQXCXDXLNXMENTQRCMCXALQXIMXXXCDXLQXXXMVQQFXXXLMN |
| | positions | LPQQCXFXAPXXCXLXVSGGXC |
| 23 | | MXXEXGXXGXSXSCXXXVXXVXLXPCEXHIMQRIXGXXEXXXSYXXXSTRSSD |
| | | QQQXCXDXLNXMENTQRCMCXALQXIMXXXCDXLQXXXMVQQFXXXLMN |
| | | LPQQCXFXAPXXCXLXVSGGXC |
| 24 | Arah6_D82 | RDEDGRAGDSSSCEDQVRRVNLDPCESHIMQRIAGAAEQDDSYDRRSTRSSD |
| | | QQQRCCDELNEMENTQRCMCEALQQIMENQCDRLQGDQMVQQFKDELM |
| | | NLPQQCDFEAPQDCKLDVSGGRC |
| 25 | Arah6_D83 | RDEDGRAGDSSSCEDQVRRVNLKPCEQHIMQRIMGEQEQDDSYDRRSTRSS |
| | | DQQQRCCDELNEMENTQRCMCEALQQIMENQCDRLQGDQMVQQFKDEL |
| | | MNLPQQCDFEAPQDCKLDVSGGRC |
| 26 | Arah6_D84 | RDEDGRAGDSSSCEDQVQRVNLDPCESHIMQRIAGAAEQYDSYDRRSTRSSD |
| | | QQQRCCDELNEMENTQRCMCEALQQIMENQCDRLQGDQMVQQFKDELM |
| | | NLPQQCDFEAPQDCKLDVSGGRC |
| 27 | Arah6_D85 | RDEDGRAGDSSSCEDQVQRVNLKPCEQHIMQRIMGEQEQYDSYDRRSTRSS |
| | | DQQQRCCDELNEMENTQRCMCEALQQIMENQCDRLQGDQMVQQFKDEL |
| | | MNLPQQCDFEAPQDCKLDVSGGRC |
| 28 | Arah6_D86 | RDEDGRAGDSSSCEDQVRRVNLDPCESHIMQRIAGAAEQDDSYDRRSTRSSD |
| | | QQQRCCDELNEMENTQRCMCEALQQIMENQCDRLQGDQMVQQFKLELM |
| | | NLPQQCDFEAPQDCKLRVSGGRC |
| 29 | Arah6_D87 | RDEDGRAGDSSSCEDQVRRVNLKPCEQHIMQRIMGEQEQDDSYDRRSTRSS |
| | | DQQQRCCDELNEMENTQRCMCEALQQIMENQCDRLQGDQMVQQFKLEL |
| | | MNLPQQCDFEAPQDCKLRVSGGRC |
| 30 | Arah6_D88 | RDEDGRAGDSSSCEDQVQRVNLDPCESHIMQRIAGAAEQYDSYDRRSTRSSD |
| | | QQQRCCDELNEMENTQRCMCEALQQIMENQCDRLQGDQMVQQFKLELM |
| | | NLPQQCDFEAPQDCKLRVSGGRC |
| 31 | Arah6_D89 | RDEDGRAGDSSSCEDQVQRVNLKPCEQHIMQRIMGEQEQYDSYDRRSTRSS |
| | | DQQQRCCDELNEMENTQRCMCEALQQIMENQCDRLQGDQMVQQFKLEL |
| | | MNLPQQCDFEAPQDCKLRVSGGRC |
| 32 | Arah6_D90 | RDEDGRAGDSSSCEDQVQRVNLDPCESHIMQRIAGAAEQDDSYDRRSTRSS |
| | | DQQQRCCDELNEMENTQRCMCEALQQIMENQCDRLQGDQMVQQFKLEL |
| | | MNLPQQCDFEAPQDCKLRVSGGRC |
| 33 | Arah6_D91 | RDEDGRAGDSSSCEDQVQRVNLKPCEQHIMQRIMGEQEQDDSYDRRSTRSS |
| | | DQQQRCCDELNEMENTQRCMCEALQQIMENQCDRLQGDQMVQQFKLEL |
| | | MNLPQQCDFEAPQDCKLRVSGGRC |
| 34 | Arah6_D92 | RDEDGRAGDSSSCEDQVRRVNLDPCESHIMQRIAGAAEQYDSYDRRSTRSSD |
| | | QQQRCCDELNEMENTQRCMCEALQQIMENQCDRLQGDQMVQQFKDELM |
| | | NLPQQCDFEAPQDCKLDVSGGRC |
| 35 | Arah6_D93 | RDEDGRAGDSSSCEDQVRRVNLKPCEQHIMQRIMGEQEQYDSYDRRSTRSS |
| | | DQQQRCCDELNEMENTQRCMCEALQQIMENQCDRLQGDQMVQQFKDEL |
| | | MNLPQQCDFEAPQDCKLDVSGGRC |
| 36 | Arah6_D94 | RDEDGRAGDSSSCEDQVRRVNLDPCESHIMQRIAGAAEQYDSYDRRSTRSSD |
| | | QQQRCCDELNEMENTQRCMCEALQQIMENQCDRLQGDQMVQQFKDELM |
| | | NLPQQCDFEAPQDCKLRVSGGRC |
| 37 | Arah6_D95 | RDEDGRAGDSSSCEDQVRRVNLKPCEQHIMQRIMGEQEQYDSYDRRSTRSS |
| | | DQQQRCCDELNEMENTQRCMCEALQQIMENQCDRLQGDQMVQQFKDEL |
| | | MNLPQQCDFEAPQDCKLRVSGGRC |
| 38 | Arah6_D96 | RDEDGRAGDSSSCEDQVQRVNLDPCESHIMQRIAGAAEQYDSYDRRSTRSSD |
| | | QQQRCCDELNEMENTQRCMCEALQQIMENQCDRLQGDQMVQQFKDELM |
| | | NLPQQCDFEAPQDCKLRVSGGRC |
| 39 | Arah6_D97 | RDEDGRAGDSSSCEDQVQRVNLKPCEQHIMQRIMGEQEQYDSYDRRSTRSS |
| | | DQQQRCCDELNEMENTQRCMCEALQQIMENQCDRLQGDQMVQQFKDEL |
| | | MNLPQQCDFEAPQDCKLRVSGGRC |
| 40 | Arah6_D98 | RDEDGRAGDSSSCEDQVRRVNLDPCESHIMQRIAGAAEQYDSYDRRSTRSSD |
| | | QQQRCCDELNEMENTQRCMCEALQQIMENQCDRLQGDQMVQQFKLELM |
| | | NLPQQCDFEAPQDCKLRVSGGRC |
| 41 | Arah6_D99 | RDEDGRAGDSSSCEDQVRRVNLKPCEQHIMQRIMGEQEQYDSYDRRSTRSS |
| | | DQQQRCCDELNEMENTQRCMCEALQQIMENQCDRLQGDQMVQQFKLEL |
| | | MNLPQQCDFEAPQDCKLRVSGGRC |
| 42 | Arah6_D100 | RDEDGRAGDSSSCEDQVQRVNLDPCESHIMQRIAGAAEQYDSYDRRSTRSSD |
| | | QQQRCCDELNEMENTQRCMCEALQQIMENQCDRLQGDQMVQQFKLELM |
| | | NLPQQCDFEAPQDCKLDVSGGRC |
| 43 | Arah6_D101 | RDEDGRAGDSSSCEDQVQRVNLKPCEQHIMQRIMGEQEQYDSYDRRSTRSS |
| | | DQQQRCCDELNEMENTQRCMCEALQQIMENQCDRLQGDQMVQQFKLEL |
| | | MNLPQQCDFEAPQDCKLDVSGGRC |
| 44 | Arah6_D113 | RDEDGRAGDSSSCEDQVQRVNLDPCESHIMQRIAGAAEQDDSYDRRSTRSS |
| | | DQQQRCCDELNEMENTQRCMCEALQQIMENQCDRLQGDQMVQQFKLEL |
| | | MNLPQQCDFEAPQDCKLDVSGGRC |
| 45 | Arah6_D114 | RDEDGRAGDSSSCEDQVQRVNLKPCEQHIMQRIMGEQEQDDSYDRRSTRSS |
| | | DQQQRCCDELNEMENTQRCMCEALQQIMENQCDRLQGDQMVQQFKLEL |
| | | MNLPQQCDFEAPQDCKLDVSGGRC |
| 46 | Arah6_D119 | RDEDGRAGDSSSCERQVRRVNLKPCEQHIMQRIAGAAEQYDSYARRSTRSSD |
| | | QQQRCCDELNEMENTQRCMCEALQQIMENQCDRLQGDQMVQQFKDELM |
| | | NLPQQCDFEAPQDCKLRVSGGRC |
| 47 | Arah6_D120 | RDEDGRAGDSSSCERQVRRVNLDPCESHIMQRIMGEQEQYDSYARRSTRSSD |
| | | QQQRCCDELNEMENTQRCMCEALQQIMENQCDRLQGDQMVQQFKDELM |
| | | NLPQQCDFEAPQDCKLRVSGGRC |
| 48 | Arah6_D121 | RDEDGRAGDSSSCERQVRRVNLKPCEQHIMQRIMGEQEQYDSYARRSTRSS |
| | | DQQQRCCDELNEMENTQRCMCEALQQIMENQCDRLQGDQMVQQFKDEL |
| | | MNLPQQCDFEAPQDCKLRVSGGRC |
| 49 | Arah6_D122 | RDEDGRAGDSSSCERQVRRVNLKPCEQHIMQRIAGAAEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 50 | Arah6_D123 | RDEDGRAGDSSSCERQVRRVNLDPCESHIMQRIMGEQEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 51 | Arah6_D124 | RDEDGRAGDSSSCERQVRRVNLKPCEQHIMQRIMGEQEQYDSYARRSTRSS |
| | | DQQQRCCDELNEMENTQRCMCEALQQIMENQCDRLQGDQMVQQFKREL |
| | | MNLPQQCDFEAPQDCKLRVSGGRC |
| 52 | Arah6_D125 | RDEDGRAGDSSSCERQVDRVNLKPCEQHIMQRIAGAAEQYDSYARRSTRSSD |
| | | QQQRCCDELNEMENTQRCMCEALQQIMENQCDRLQGDQMVQQFKDELM |
| | | NLPQQCDFEAPQDCKLRVSGGRC |
| 53 | Arah6_D126 | RDEDGRAGDSSSCERQVDRVNLDPCESHIMQRIMGEQEQYDSYARRSTRSS |
| | | DQQQRCCDELNEMENTQRCMCEALQQIMENQCDRLQGDQMVQQFKDEL |
| | | MNLPQQCDFEAPQDCKLRVSGGRC |
| 54 | Arah6_D127 | RDEDGRAGDSSSCERQVDRVNLKPCEQHIMQRIMGEQEQYDSYARRSTRSS |
| | | DQQQRCCDELNEMENTQRCMCEALQQIMENQCDRLQGDQMVQQFKDEL |
| | | MNLPQQCDFEAPQDCKLRVSGGRC |
| 55 | Arah6_D128 | RDEDGRAGDSSSCERQVDRVNLKPCEQHIMQRIAGAAEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 56 | Arah6_D129 | RDEDGRAGDSSSCERQVDRVNLDPCESHIMQRIMGEQEQYDSYARRSTRSS |
| | | DQQQRCCDELNEMENTQRCMCEALQQIMENQCDRLQGDQMVQQFKREL |
| | | MNLPQQCDFEAPQDCKLRVSGGRC |
| 57 | Arah6_D130 | RDEDGRAGDSSSCERQVDRVNLKPCEQHIMQRIMGEQEQYDSYARRSTRSS |
| | | DQQQRCCDELNEMENTQRCMCEALQQIMENQCDRLQGDQMVQQFKREL |
| | | MNLPQQCDFEAPQDCKLRVSGGRC |
| 58 | Arah6_D131 | RDEDGRAGDSSSCERQVRRVNLDPCESHIMQRIAGAAEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 59 | Arah6_D132 | RDEDGRAGDSSSCERQVDRVNLDPCESHIMQRIAGAAEQYDSYARRSTRSSD |
| | | QQQRCCDELNEMENTQRCMCEALQQIMENQCDRLQGDQMVQQFKDELM |
| | | NLPQQCDFEAPQDCKLRVSGGRC |
| 60 | Arah6_D133 | RDEDGRAGDSSSCERQVRRVNLDPCESHIMQRIAGAAEQYDSYARRSTRSSD |
| | | QQQRCCDELNEMENTQRCMCEALQQIMENQCDRLQGDQMVQQFKDELM |
| | | NLPQQCDFEAPQDCKLRVSGGRC |
| 61 | Arah6_D134 | RDEDGRAGDSSSCERQVDRVNLKPCESHIMQRIAGAAEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 62 | Arah6_D135 | RDEDGRAGDSSSCERQVDRVNLDPCEQHIMQRIAGAAEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 63 | Arah6_D136 | RDEDGRAGDSSSCERQVDRVNLDPCESHIMQRIMGAAEQYDSYARRSTRSS |
| | | DQQQRCCDELNEMENTQRCMCEALQQIMENQCDRLQGDQMVQQFKREL |
| | | MNLPQQCDFEAPQDCKLRVSGGRC |
| 64 | Arah6_D137 | RDEDGRAGDSSSCERQVDRVNLDPCESHIMQRIAGEAEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 65 | Arah6_D138 | RDEDGRAGDSSSCERQVDRVNLDPCESHIMQRIAGAQEQYDSYARRSTRSSD |
| | | QQQRCCDELNEMENTQRCMCEALQQIMENQCDRLQGDQMVQQFKRELM |
| | | NLPQQCDFEAPQDCKLRVSGGRC |
| 66 | Arah6_D144 | RDEDGRAGDSSSCERQVRRVNLKPCEQHIMQRIAGAAEQYDSYARRSTRSSD |
| | | QQQRCCDELNEMENTQRCMCEALQQIMENQCDDLQGDQMVQQFKDELM |
| | | NLPQQCDFEAPQDCKLRVSGGRC |
| 67 | Arah6_D145 | RDEDGRAGDSSSCERQVRRVNLKPCEQHIMQKIAGAAEQYDSYARRSTRSSD |
| | | QQQRCCDELNEMENTQRCMCEALQQIMENQCDRLQGDQMVQQFKDELM |
| | | NLPQKCDFEAPQDCKLRVSGGRC |
| 68 | Arah6_D146 | RDEDGRAGDSSSCERQVRRVNLKPCEQHIMQKIAGAAEQYDSYARRSTRSSD |
| | | QQQRCCDELNEMENTQRCMCEALQQIMENQCDDLQGDQMVQQFKDELM |
| | | NLPQKCDFEAPQDCKLRVSGGRC |
| 69 | Arah6_D147 | RDEDGRAGDSSSCERQVRRVNLKPCEQHIMQQIAGAAEQYDSYARRSTRSSD |
| | | QQQRCCDELNEMENTQRCMCEALQQIMENQCDRLQGDQMVQQFKDELM |
| | | NLPQKCDFEAPQDCKLRVSGGRC |
| 70 | Arah6_D148 | RDEDGRAGDSSSCERQVRRVNLKPCEQHIMQQIAGAAEQYDSYARRSTRSSD |
| | | QQQRCCDELNEMENTQRCMCEALQQIMENQCDDLQGDQMVQQFKDELM |
| | | NLPQKCDFEAPQDCKLRVSGGRC |
| 71 | Arah6_D149 | RDEDGRAGDSSSCERQVRRVNLKPCEQHIMQKIAGAAEQYDSYARRSTRSSD |
| | | QQQRCCDELNEMENTQRCMCEALQQIMENQCDRLQGDQMVQQFKDELM |
| | | NLPQHCDFEAPQDCKLRVSGGRC |
| 72 | Arah6_D150 | RDEDGRAGDSSSCERQVRRVNLKPCEQHIMQKIAGAAEQYDSYARRSTRSSD |
| | | QQQRCCDELNEMENTQRCMCEALQQIMENQCDDLQGDQMVQQFKDELM |
| | | NLPQHCDFEAPQDCKLRVSGGRC |
| 73 | Arah6_D151 | RDEDGRAGDSSSCERQVRRVNLKPCEQHIMQQIAGAAEQYDSYARRSTRSSD |
| | | QQQRCCDELNEMENTQRCMCEALQQIMENQCDRLQGDQMVQQFKDELM |
| | | NLPQHCDFEAPQDCKLRVSGGRC |
| 74 | Arah6_D152 | RDEDGRAGDSSSCERQVRRVNLKPCEQHIMQQIAGAAEQYDSYARRSTRSSD |
| | | QQQRCCDELNEMENTQRCMCEALQQIMENQCDDLQGDQMVQQFKDELM |
| | | NLPQHCDFEAPQDCKLRVSGGRC |
| 75 | Arah6_D153 | RDEDGRAGDSSSCERQVRRVNLKPCEQHIMQRIAGAAEQYDSYARRSTRSSD |
| | | QQQRCCDELNEMENTQRCMCEALQQIMENQCDRLQGDQMVQQFKDELM |
| | | NLPQKCDFEAPQDCKLRVSGGRC |
| 76 | Arah6_D154 | RDEDGRAGDSSSCERQVRRVNLKPCEQHIMQRIAGAAEQYDSYARRSTRSSD |
| | | QQQRCCDELNEMENTQRCMCEALQQIMENQCDDLQGDQMVQQFKDELM |
| | | NLPQKCDFEAPQDCKLRVSGGRC |
| 77 | Arah6_D155 | RDEDGRAGDSSSCERQVRRVNLKPCEQHIMQRIMGAAEQYDSYARRSTRSS |
| | | DQQQRCCDELNEMENTQRCMCEALQQIMENQCDRLQGDQMVQQFKDEL |
| | | MNLPQQCDFEAPQDCKLRVSGGRC |
| 78 | Arah6_D156 | RDEDGRAGDSSSCERQVRRVNLKPCEQHIMQRIAGEAEQYDSYARRSTRSSD |
| | | QQQRCCDELNEMENTQRCMCEALQQIMENQCDRLQGDQMVQQFKDELM |
| | | NLPQQCDFEAPQDCKLRVSGGRC |
| 79 | Arah6_D157 | RDEDGRAGDSSSCERQVRRVNLKPCEQHIMQRIAGAQEQYDSYARRSTRSSD |
| | | QQQRCCDELNEMENTQRCMCEALQQIMENQCDRLQGDQMVQQFKDELM |
| | | NLPQQCDFEAPQDCKLRVSGGRC |
| 80 | Arah6_D158 | RDEDGRAGDSSSCERQVRRVNLKPCEQHIMQRIMGEQEQYDSYARRSTRSS |
| | | DQQQRCCDELNEMENTQRCMCEALQQIMENQCDDLQGDQMVQQFKDEL |
| | | MNLPQKCDFEAPQDCKLRVSGGRC |
| 81 | Arah6_D159 | RDEDGRAGDSSSCERQVRRVNLKPCEQHIMQRIAGEQEQYDSYARRSTRSSD |
| | | QQQRCCDELNEMENTQRCMCEALQQIMENQCDDLQGDQMVQQFKDELM |
| | | NLPQKCDFEAPQDCKLRVSGGRC |
| 82 | Arah6_D160 | RDEDGRAGDSSSCERQVRRVNLKPCEQHIMQRIMGAQEQYDSYARRSTRSS |
| | | DQQQRCCDELNEMENTQRCMCEALQQIMENQCDDLQGDQMVQQFKDEL |
| | | MNLPQKCDFEAPQDCKLRVSGGRC |
| 83 | Arah6_D161 | RDEDGRAGDSSSCERQVRRVNLKPCEQHIMQRIMGEAEQYDSYARRSTRSSD |
| | | QQQRCCDELNEMENTQRCMCEALQQIMENQCDDLQGDQMVQQFKDELM |
| | | NLPQKCDFEAPQDCKLRVSGGRC |
| 84 | Arah6_D162 | RDEDGRAGDSSSCERQVRRVNLKPCEQHIMQRIMGAAEQYDSYARRSTRSS |
| | | DQQQRCCDELNEMENTQRCMCEALQQIMENQCDDLQGDQMVQQFKDEL |
| | | MNLPQKCDFEAPQDCKLRVSGGRC |
| 85 | Arah6_D163 | RDEDGRAGDSSSCERQVRRVNLKPCEQHIMQRIAGEAEQYDSYARRSTRSSD |
| | | QQQRCCDELNEMENTQRCMCEALQQIMENQCDDLQGDQMVQQFKDELM |
| | | NLPQKCDFEAPQDCKLRVSGGRC |
| 86 | Arah6_D164 | RDEDGRAGDSSSCERQVRRVNLKPCEQHIMQRIAGAQEQYDSYARRSTRSSD |
| | | QQQRCCDELNEMENTQRCMCEALQQIMENQCDDLQGDQMVQQFKDELM |
| | | NLPQKCDFEAPQDCKLRVSGGRC |
| 87 | Arah6_D165 | RDEDGRAGDSSSCERQVRRVNLKPCEQHIMQRIMGEQEQYDSYARRSTRSS |
| | | DQQQRCCDELNEMENTQRCMCEALQQIMENQCDDLQGRQMVQQFKDEL |
| | | MNLPQKCDFEAPQDCKLRVSGGRC |
| 88 | Arah6_D166 | RDEDGRAGDSSSCERQVRRVNLKPCEQHIMQRIAGEQEQYDSYARRSTRSSD |
| | | QQQRCCDELNEMENTQRCMCEALQQIMENQCDDLQGRQMVQQFKDELM |
| | | NLPQKCDFEAPQDCKLRVSGGRC |
| 89 | Arah6_D167 | RDEDGRAGDSSSCERQVRRVNLKPCEQHIMQRIMGAQEQYDSYARRSTRSS |
| | | DQQQRCCDELNEMENTQRCMCEALQQIMENQCDDLQGRQMVQQFKDEL |
| | | MNLPQKCDFEAPQDCKLRVSGGRC |
| 90 | Arah6_D168 | RDEDGRAGDSSSCERQVRRVNLKPCEQHIMQRIMGEAEQYDSYARRSTRSSD |
| | | QQQRCCDELNEMENTQRCMCEALQQIMENQCDDLQGRQMVQQFKDELM |
| | | NLPQKCDFEAPQDCKLRVSGGRC |
| 91 | Arah6_D169 | RDEDGRAGDSSSCERQVRRVNLKPCEQHIMQRIMGAAEQYDSYARRSTRSS |
| | | DQQQRCCDELNEMENTQRCMCEALQQIMENQCDDLQGRQMVQQFKDEL |
| | | MNLPQKCDFEAPQDCKLRVSGGRC |
| 92 | Arah6_D170 | RDEDGRAGDSSSCERQVRRVNLKPCEQHIMQRIAGEAEQYDSYARRSTRSSD |
| | | QQQRCCDELNEMENTQRCMCEALQQIMENQCDDLQGRQMVQQFKDELM |
| | | NLPQKCDFEAPQDCKLRVSGGRC |
| 93 | Arah6_D171 | RDEDGRAGDSSSCERQVRRVNLKPCEQHIMQRIAGAQEQYDSYARRSTRSSD |
| | | QQQRCCDELNEMENTQRCMCEALQQIMENQCDDLQGRQMVQQFKDELM |
| | | NLPQKCDFEAPQDCKLRVSGGRC |
| 94 | Arah6_D172 | RDEDGRAGDSSSCERQVRRVNLKPCEQHIMQRIMGEQEQYDSYARRSTRSS |
| | | DQQQRCCDELNEMENTQRCMCEALQQIMENQCDDLQGDQMVQQFKDEL |
| | | MNLPQKCNFEAPQDCKLRVSGGRC |
| 95 | Arah6_D173 | RDEDGRAGDSSSCERQVRRVNLKPCEQHIMQRIAGEQEQYDSYARRSTRSSD |
| | | QQQRCCDELNEMENTQRCMCEALQQIMENQCDDLQGDQMVQQFKDELM |
| | | NLPQKCNFEAPQDCKLRVSGGRC |
| 96 | Arah6_D174 | RDEDGRAGDSSSCERQVRRVNLKPCEQHIMQRIMGAQEQYDSYARRSTRSS |
| | | DQQQRCCDELNEMENTQRCMCEALQQIMENQCDDLQGDQMVQQFKDEL |
| | | MNLPQKCNFEAPQDCKLRVSGGRC |
| 97 | Arah6_D175 | RDEDGRAGDSSSCERQVRRVNLKPCEQHIMQRIMGEAEQYDSYARRSTRSSD |
| | | QQQRCCDELNEMENTQRCMCEALQQIMENQCDDLQGDQMVQQFKDELM |
| | | NLPQKCNFEAPQDCKLRVSGGRC |
| 98 | Arah6_D176 | RDEDGRAGDSSSCERQVRRVNLKPCEQHIMQRIMGAAEQYDSYARRSTRSS |
| | | DQQQRCCDELNEMENTQRCMCEALQQIMENQCDDLQGDQMVQQFKDEL |
| | | MNLPQKCNFEAPQDCKLRVSGGRC |
| 99 | Arah6_D177 | RDEDGRAGDSSSCERQVRRVNLKPCEQHIMQRIAGEAEQYDSYARRSTRSSD |
| | | QQQRCCDELNEMENTQRCMCEALQQIMENQCDDLQGDQMVQQFKDELM |
| | | NLPQKCNFEAPQDCKLRVSGGRC |
| 100 | Arah6_D178 | RDEDGRAGDSSSCERQVRRVNLKPCEQHIMQRIAGAQEQYDSYARRSTRSSD |
| | | QQQRCCDELNEMENTQRCMCEALQQIMENQCDDLQGDQMVQQFKDELM |
| | | NLPQKCNFEAPQDCKLRVSGGRC |
| 101 | Arah6_D179 | RDEDGRAGDSSSCERQVRRVNLKPCEQHIMQRIMGEQEQYDSYARRSTRSS |
| | | DQQQRCCDELNEMENTQRCMCEALQQIMENQCDDLQGRQMVQQFKDEL |
| | | MNLPQKCNFEAPQDCKLRVSGGRC |
| 102 | Arah6_D180 | RDEDGRAGDSSSCERQVRRVNLKPCEQHIMQRIAGEQEQYDSYARRSTRSSD |
| | | QQQRCCDELNEMENTQRCMCEALQQIMENQCDDLQGRQMVQQFKDELM |
| | | NLPQKCNFEAPQDCKLRVSGGRC |
| 103 | Arah6_D181 | RDEDGRAGDSSSCERQVRRVNLKPCEQHIMQRIMGAQEQYDSYARRSTRSS |
| | | DQQQRCCDELNEMENTQRCMCEALQQIMENQCDDLQGRQMVQQFKDEL |
| | | MNLPQKCNFEAPQDCKLRVSGGRC |
| 104 | Arah6_D182 | RDEDGRAGDSSSCERQVRRVNLKPCEQHIMQRIMGEAEQYDSYARRSTRSSD |
| | | QQQRCCDELNEMENTQRCMCEALQQIMENQCDDLQGRQMVQQFKDELM |
| | | NLPQKCNFEAPQDCKLRVSGGRC |
| 105 | Arah6_D183 | RDEDGRAGDSSSCERQVRRVNLKPCEQHIMQRIMGAAEQYDSYARRSTRSS |
| | | DQQQRCCDELNEMENTQRCMCEALQQIMENQCDDLQGRQMVQQFKDEL |
| | | MNLPQKCNFEAPQDCKLRVSGGRC |
| 106 | Arah6_D184 | RDEDGRAGDSSSCERQVRRVNLKPCEQHIMQRIAGEAEQYDSYARRSTRSSD |
| | | QQQRCCDELNEMENTQRCMCEALQQIMENQCDDLQGRQMVQQFKDELM |
| | | NLPQKCNFEAPQDCKLRVSGGRC |
| 107 | Arah6_D185 | RDEDGRAGDSSSCERQVRRVNLKPCEQHIMQRIAGAQEQYDSYARRSTRSSD |
| | | QQQRCCDELNEMENTQRCMCEALQQIMENQCDDLQGRQMVQQFKDELM |
| | | NLPQKCNFEAPQDCKLRVSGGRC |
| 108 | Arah6_D186 | RDEDGRAGDSSSCERQVRRVNLKPCEQHIMQRIAGAAEQYDSYARRSTRSSD |
| | | QQQRCCDELNEMENTQRCMCEALQQIMENQCDDLQGRQMVQQFKDELM |
| | | NLPQQCDFEAPQRCKLRVSGGRC |
| 109 | | MXXEXGXXGXSXSCXXXVXXVXLXPCEXHIMQXIXGXXEXXXSYXXXSTRSS |
| | | DQQQXCXDXLXXMENTQRCMCXALQXIMXXXCDXLQXXXMVQQFXXXLMNLP |
| | | QXCXXXAPXXCXLXVSGGXC |
| 110 | | MXXEXGXXGXSXSCXXXVXXVXLXPCEXHIMQXIXGXXEXXXSYXXXSTRSS |
| | | DQQQXCXDXLNXMENTQRCMCXALQXIMXXXCDXLQXXXMVQQFXXXLMNLP |
| | | QXCXFXAPXXCXLXVSGGXC |

### Summary

Following the above procedures, 7 Ara h 6 epitopes were found.

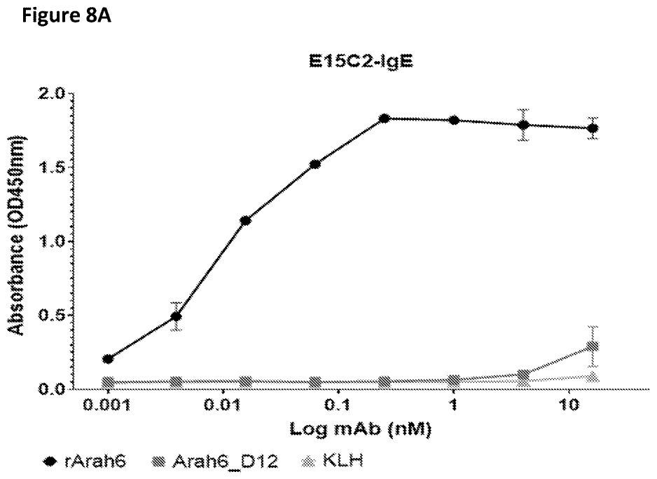

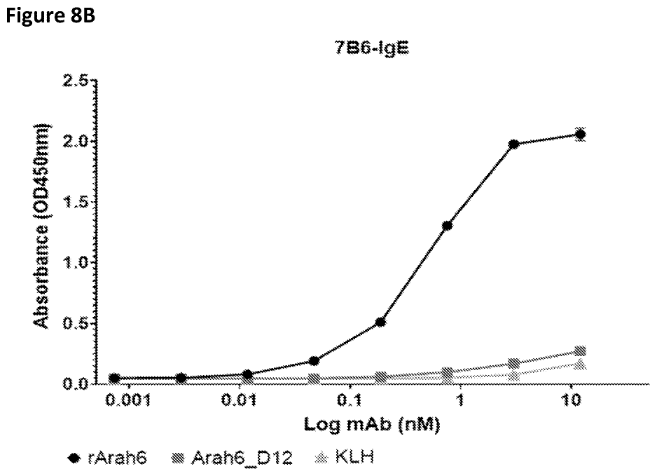

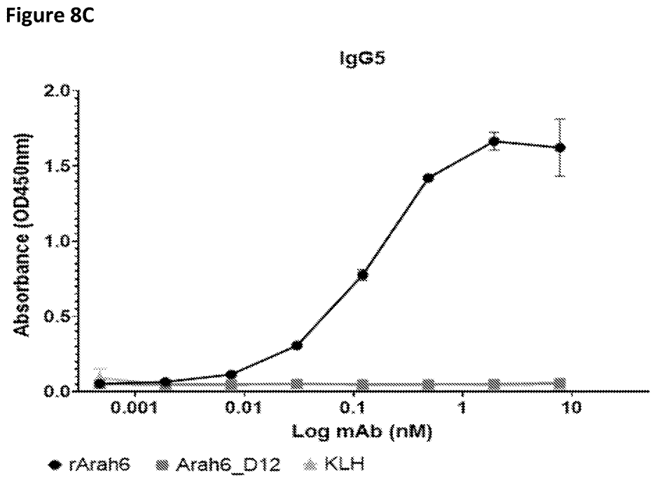

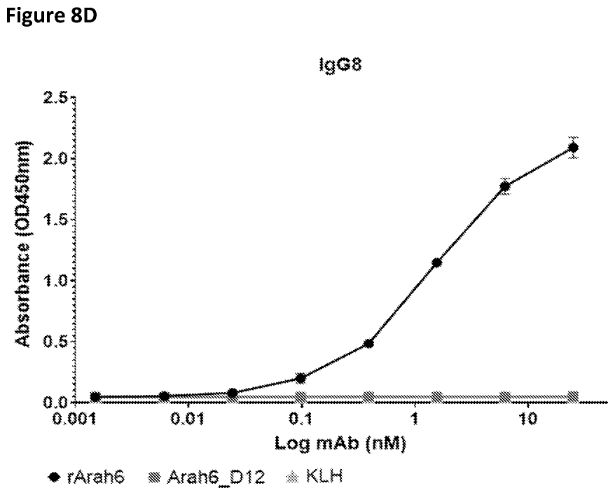

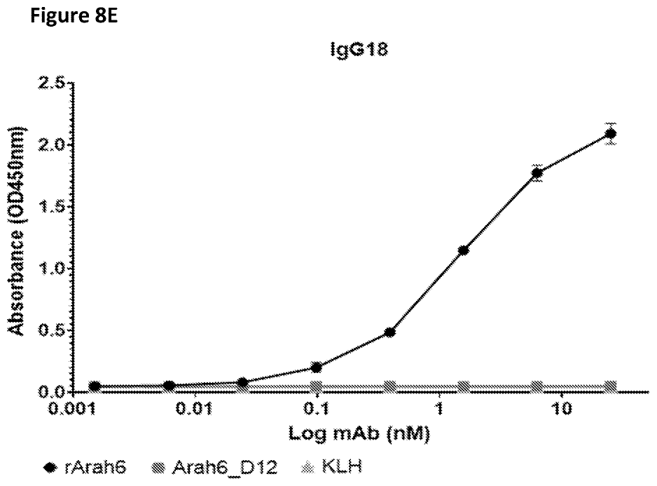

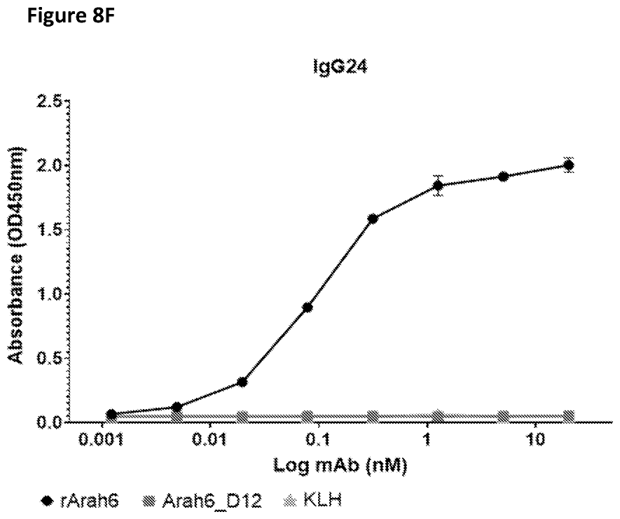

Ara h 6 D12 variant shows reduced binding to anti-Ara h 6 mAbs, 10 IgGs and 2 IgEs. Indirect ELISA titration with increasing concentrations of the anti Ara h 6 mAb was used to test binding to WT recombinant Ara h 6 (SEQ ID NO: 2) or modified Ara h 6 D12 variant (SEQ ID NO: 9), keyhole limpet hemocyanin (KLH) was used as a negative control. The data presented demonstrates that modified Ara h 6 D12 variant shows dramatically reduced binding to 2 anti-Ara h 6 IgEs (E15C2 and 7B6) and 4 anti-Ara h 6 IgGs (IgG5, IgG8, IgG18 and IgG24) (FIGS. 8A-8F).

### Example 5: Allergenicity Assessment of Engineered Proteins by Ex-Vivo Basophil Degranulation Assays

Objective: To assess the allergenicity of the engineered Ara h 6 variants relative to wild-type proteins.

### Results

Based on the results from the single-site linear and conformational de-epitoping seen in Examples 2-4, mutations that abolish the binding to each epitope were combined to construct Ara h 6 variants (SEQ ID NOs: 3-21 and SEQ ID NOs: 24-108; detailed in Table 2) mutated at multiple binding sites. Alternatively, additional sequences have been computationally combined by a Monte-Carlo procedure, starting from residue level data and yielding protein variants mutated at multiple sites. The mutations listed in Table 1 above summarize the individual mutation sites.

In some embodiments of the Ara h 6 variants, the amino acid sequence set forth in any of SEQ ID NOs: 3-21 further comprises a methionine located upstream of the N terminus of the amino acid sequence set forth in any of SEQ ID NOs: 3-21. In some embodiments of the Ara h 6 variants, the amino acid sequence set forth in any of SEQ ID NOs: 24-108 further comprises a methionine located upstream of the N terminus of the amino acid sequence set forth in any of SEQ ID NOs: 24-108.

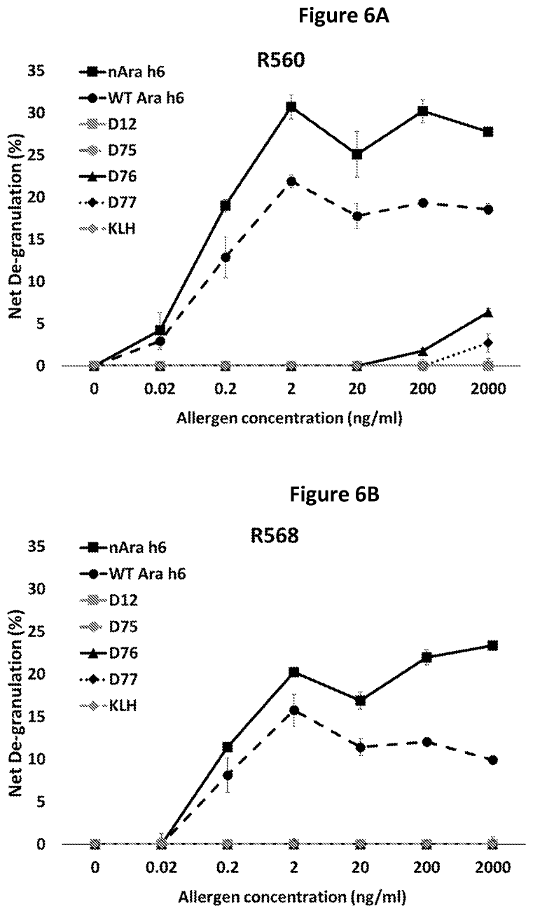

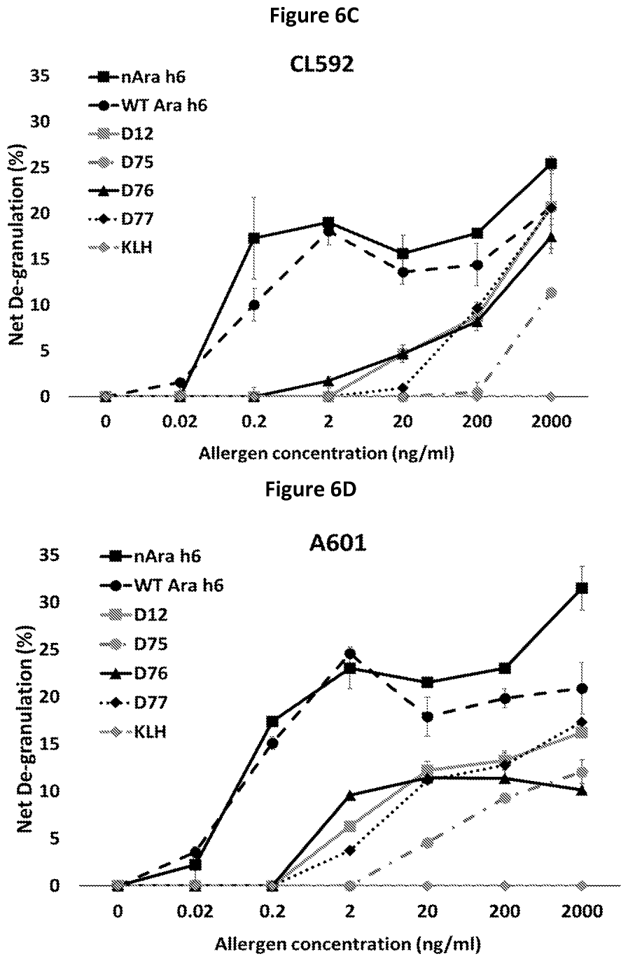

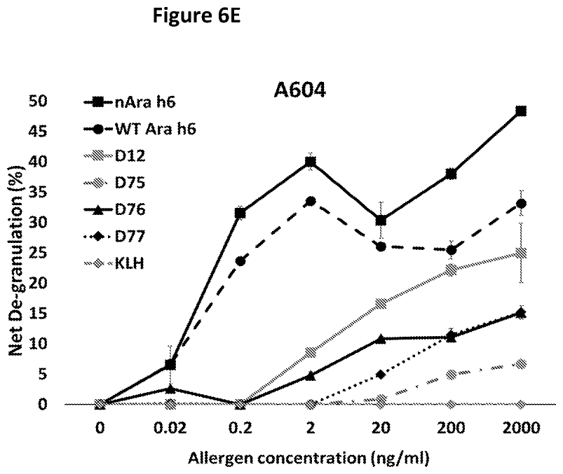

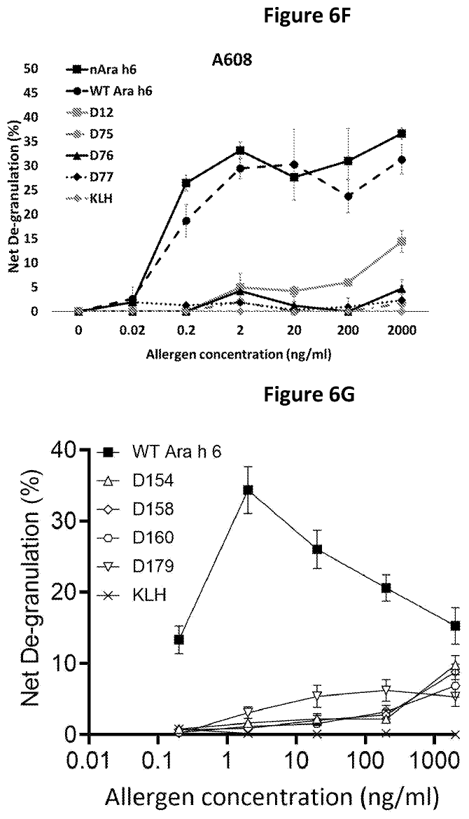

This process yielded variants that showed reduced allergenic potential compared to the WT protein. These engineered recombinant variants were expressed in E. coli, purified and tested for allergenicity. Testing was first performed on a wide ensemble of variants with a cell degranulation assay using a humanized Rat Basophil Leukemia cell line (RBL SX-38) that was sensitized with peanut allergy patient plasma. Representative results from RBL assays for Ara h 6 variants are shown in FIGS. 6A-6G. The variant allergens elicit clearly reduced cellular degranulation compared to the WT and natural allergens.

### Summary

Based on RBL ex-vivo assays, potential abrogation of allergenicity was observed for multiple Ara h 6 mutated variants that harbor combinations of mutations at more than one epitope.

### Example 6: Immunogenicity Assessment

Objective: To assess the immunogenicity of representative Ara h 6 variants.

In order to guarantee immunotherapeutic efficacy, the recombinant engineered hypoallergenic variants should substantially retain immunogenicity that would enable reprogramming of the immune response. To assess the immunogenicity level of the Ara h 6 variants, various known in the art techniques can be used such as T-cell assay, animal models, IgE/IgG binding ratios.

### Example 7: Biophysical Characteristics of the Variants

Objective: It is important to maintain the same oligomerization level of the natural proteins (i.e., monomer for Ara h 6) to ensure the correct 3D folding in the mutated variants. In order to validate the oligomerization state of the proteins, size-exclusion chromatography (SEC) HPLC was performed on each variant and only variants with the correct oligomerization state were considered valid candidates for hypoallergenic variant development (data not shown).

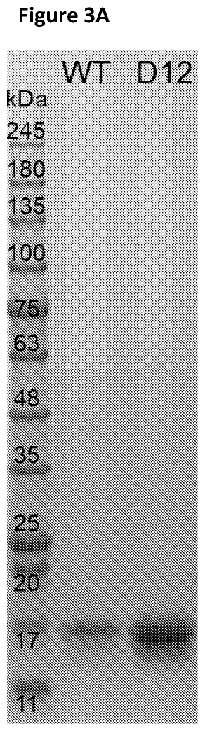

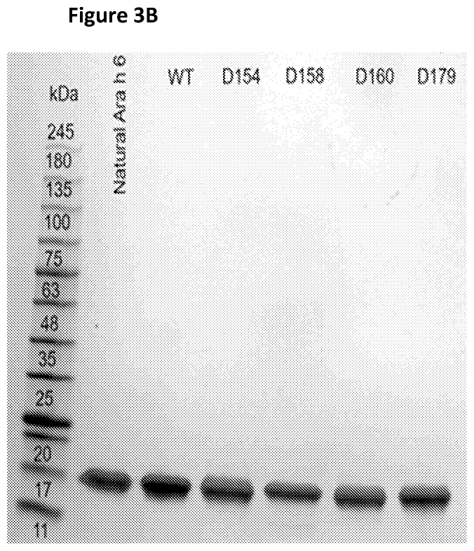

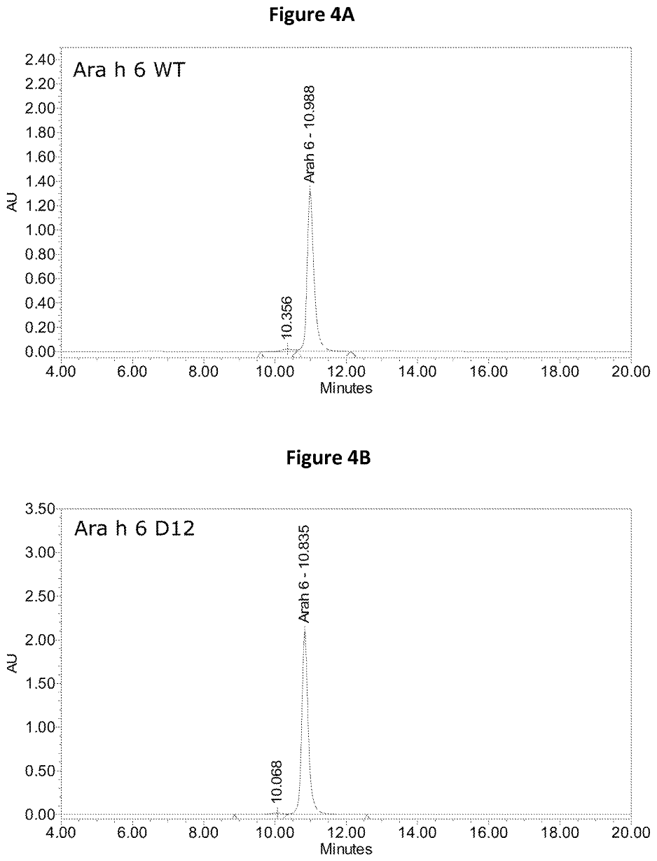

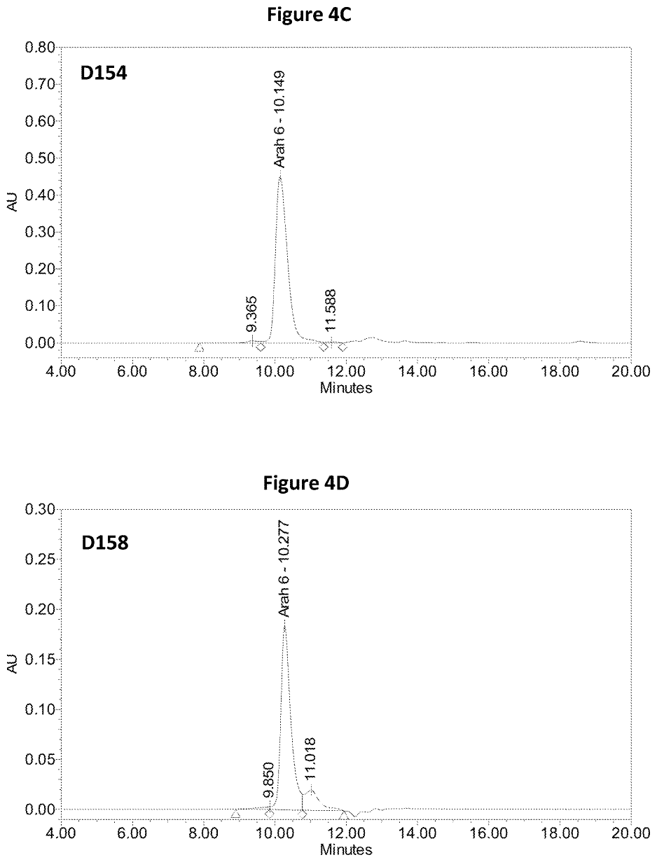

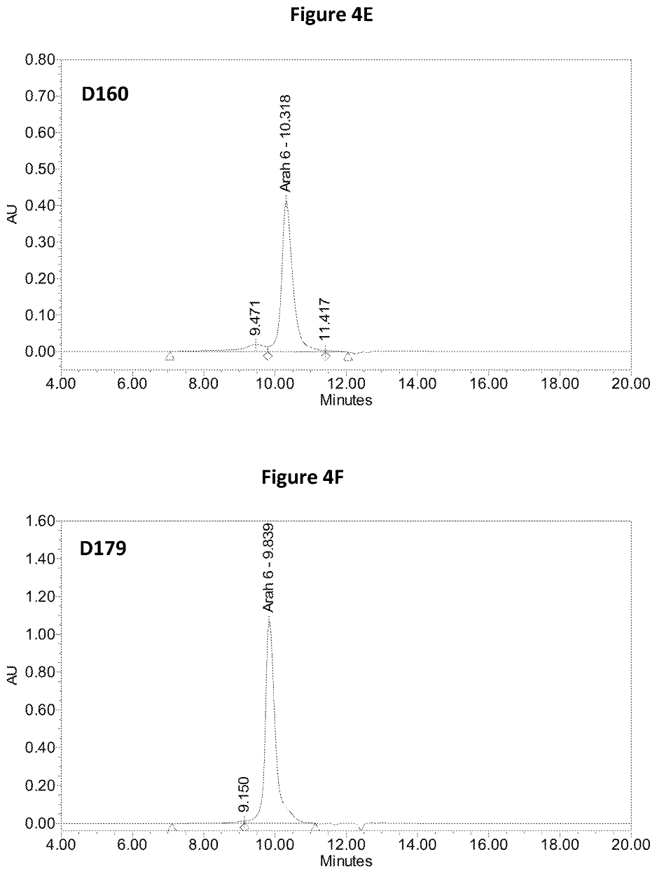

Both WT and D12 variant of Ara h 6 with a 6×Histidine tag, expressed in E. coli BL21 (DE3), were purified using standard immobilized metal affinity chromatography (IMAC) and SEC. The two purified Ara h 6 proteins with a 6×Histidine tag, WT and D12 variant, which run as ~17 kDa in size on SDS-PAGE under reducing conditions, (FIG. 3A), and Ara h 6 variants D154, D158, D160 and D179 (FIG. 3B), and D119 (data not shown) are stable and run in the monomeric form under the standard conditions tested as seen in SEC-HPLC analysis (FIGS. 4A-4F).

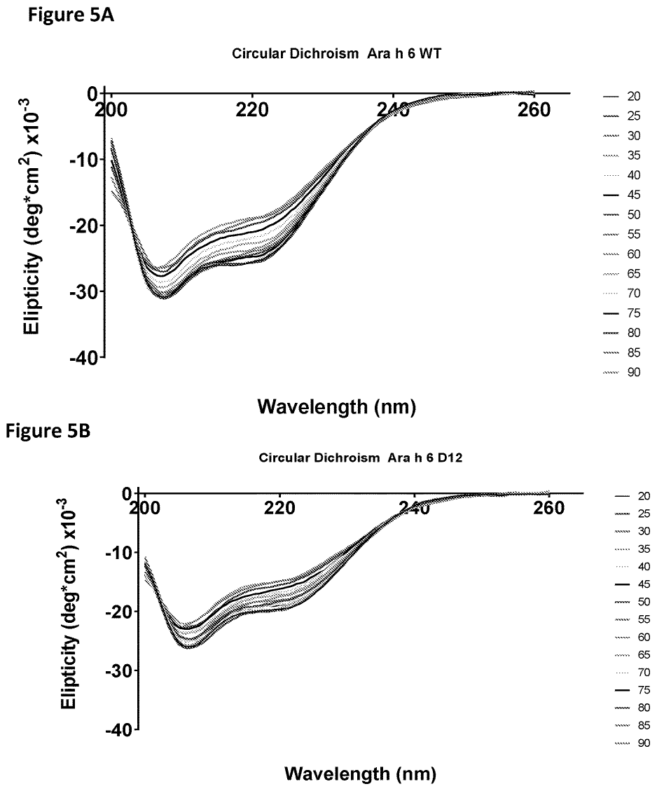

Some of the leading Ara h 6 variants were further analyzed for thermal stability using Circular Dichroism. The thermal melting Mid-Point™ of both D12 variant and the WT were >90 °C (FIGS. 5A-5B). The spectral minima of the intact constructs at ~205-210 nm and 220 nm indicate that both Ara h 6 WT and D12 are largely alpha-helical and share similar thermal stability at temperatures up to 90 °C suggesting high stability and correct fold.

### Summary

The leading Ara h 6 variants exhibit a high melting point in CD, suggesting thermal stability that is similar to the WT allergen. The combination variants of Ara h 6 were tested in SEC HPLC and present monomeric mass (~17 kDa) suggesting correct fold.

While certain features of the variant hypoallergenic peanut allergens Ara h 6 have been illustrated and described herein, many modifications, substitutions, changes, and equivalents will now occur to those of ordinary skill in the art. It is, therefore, to be understood that the appended claims are intended to cover all such modifications and changes as fall within the true spirit of these variants and uses thereof.

### Example 8: Expression and Secretion of Allergen Variants from Mammalian Cells

Objective: To demonstrate that Ara h 6 peanut proteins and de-epitoped (DE) allergen can be expressed, folded and secreted from mammalian cells.

### Methods

20 ml of Expi293F (ThermoFisher Scientific) cells were transfected with 20 micrograms of plasmid encoding for the either Ara h 6 WT, or Ara h 6 D12, both constructs expressing downstream of a human osteonectin leader sequence and containing a C terminal 6× his tag, using Expifectamine293 transfection reagent according to the manufacturer's instructions. The cells were left to express the protein for 5 days in Expi293 medium at 37 degrees, 8% CO₂. The secreted protein was purified from the expression medium using Ni-NTA superflow beads, washed and eluted with the addition 0f 350 mM imidazole. The eluted fractions were analyzed by SDS PAGE, either reduced by β-mercaptoethanol (β-ME) or non-reduced.

### Results

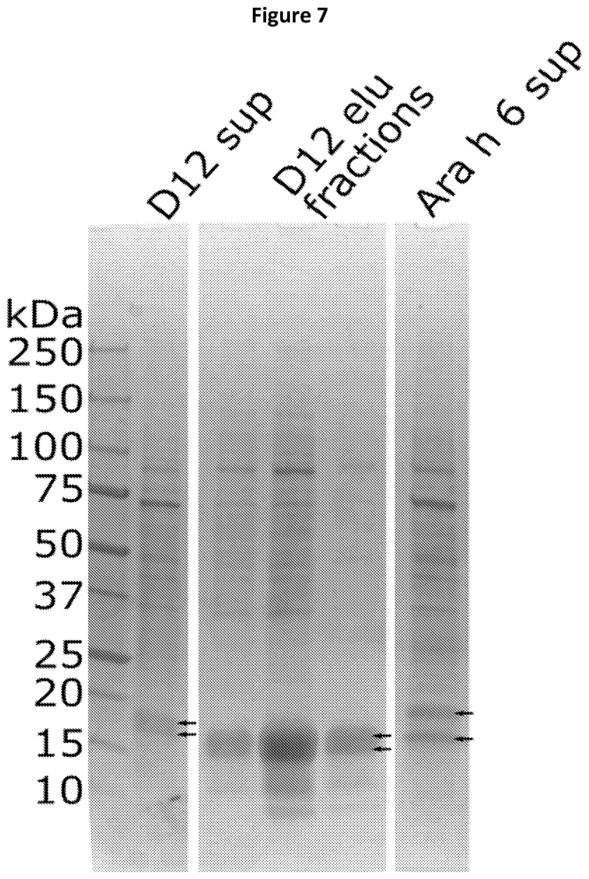

The results demonstrate that the peanut allergens Arah 6, and their de-epitoped variants can be expressed at high levels. FIG. 7 shows that wild-type or de-epitoped peanut allergen Ara h 6 were expressed and secreted from transfected mammalian cells. Purified Ara h 6 from transfected mammalian cells was found to have the correct size.

1. A recombinant Ara h 6 variant polypeptide comprising one or more amino acid substitutions, deletions, insertions, or any combination thereof, that are located at one or more of positions 2, 3, 5, 7, 8, 10, 12, 16, 19, 22, 24, 33, 37, 38, 40, 41, 42, 45, 46, 47, 63, 74, 78, 81, 82, 83, 86, 89, 90, 97, 98, 99, 106, 108, 109, 110, 113, 114, 116, or 118 of SEQ ID NO: 109, as compared with the amino acid residues at those same positions in SEQ ID NO: 2.

2. The recombinant Ara h 6 variant of claim 1, wherein the substitution comprises one or more of:
(a) S at position 2;
(b) D, or S at position 3;
(c) D at position 5;
(d) D at position 7;
(e) A, or S at position 8;
(f) A, S, or K at position 10;
(g) R, D, or N at position 12;
(h) S, or D at position 16;
(i) Q, L, or R at position 19;
(j) F at position 22;
(k) D at position 24;
(l) Q, or K at position 33;
(m) A, T, or S at position 37;
(n) A, or S at position 38;
(o) S at position 40;
(p) D at position 41;
(q) K, E, or G at position 42;
(r) A, or Q at position 45;
(s) S, G, or R at position 46;
(t) S at position 47;
(u) R at position 74;
(v) L at position 78;
(w) A, or R at position 81;
(x) T at position 82;
(y) N, or K at position 83;
(z) D, or S at position 86;
(aa) N, R, or G at position 89;
(bb) D at position 90;
(cc) I at position 97;
(dd) D, or L at position 98;
(ee) M at position 99;
(ff) K, or H at position 106;
(gg) P, E, or D at position 108;
(hh) E, or S at position 110;
(ii) D, or I at position 113;
(jj) D, H, A, or G at position 114;
(kk) K, or M at position 116; or
(ll) R, or T at position 118.

3. The recombinant Ara h 6 variant of claim 1, wherein said variant comprises amino acid substitutions, deletions, insertions, or any combination thereof, that are located at positions 3, 5, 8, 19, 45, 46, 89, 98, 110, 114, 116, and 118 of SEQ ID NO: 109, as compared with the amino acid residues at those same positions in SEQ ID NO: 2.

4. (canceled)

5. The recombinant Ara h 6 variant of claim 3, wherein said variant further comprises amino acid substitutions, deletions, insertions, or any combination thereof, at one or more of positions 2, 7, 10, 12, 15, 16, 17, 20, 22, 24, 28, 33, 35, 37, 38, 40, 41, 42, 47, 57, 59, 61, 63, 64, 74, 78, 81, 82, 83, 86, 90, 91, 97, 99, 106, 108, 109, 113, or 123 of SEQ ID NO: 109, as compared with the amino acid residues at those same positions in SEQ ID NO: 2.

6. The recombinant Ara h 6 variant of claim 5, wherein the substitutions comprise one or more of:
(a) S at position 2;
(b) D at position 7;
(c) A, S, or K at position 10;
(d) R, D, or N at position 12;
(e) R at position 15;
(f) S, or D at position 16;
(g) R at position 17;
(h) D at position 20;
(i) F at position 22;
(j) D at position 24;
(k) S at position 28;
(l) Q, or K at position 33;
(m) A at position 35;
(n) A, T, or S at position 37;
(o) A, or S at position 38;
(p) S at position 40;
(q) D at position 41;
(r) K, E, or G at position 42;
(s) S at position 47;
(t) D at position 57;
(u) Y at position 59;
(v) F at position 61;
(w) S at position 64;
(x) R at position 74;
(y) L at position 78;
(z) A, or R at position 81;
(aa) T at position 82;
(bb) N, or K at position 83;
(cc) S, or D at position 86;
(dd) D at position 90;
(ee) A, or S at position 91;
(ff) I at position 97;
(gg) M at position 99;
(hh) K, or H at position 106;
(ii) P, E, or D at position 108;
(jj) D, or I at position 113; and
(kk) D at position 123.

7. The recombinant Ara h 6 variant of claim 1, wherein said variant comprises one or more amino acid substitutions, deletions, insertions, or any combination thereof that are located within at least a single epitope recognized by an anti-Ara h 6 antibody.

8. The recombinant Ara h 6 variant of claim 1, wherein said variant comprises an amino acid sequence that is at least 80% identical to the amino acid sequence as set forth in any of SEQ ID NOs: 3-21 or SEQ ID NOs: 24-108.

9. The recombinant Ara h 6 variant of claim 1, wherein said variant comprises the amino acid sequence as set forth in any of SEQ ID NOs: 3-21 or SEQ ID NOs: 24-108.

10. An isolated nucleotide or modified nucleotide sequence encoding the recombinant Ara h 6 variant of claim 1, wherein the nucleotide or modified nucleotide sequence is DNA or mRNA.

11. The nucleotide or modified nucleotide sequence of claim 10, wherein said mRNA comprises LNP-formulated mRNA.

12. An expression vector comprising the isolated nucleotide or modified nucleotide sequence of claim 10.

13. A prokaryotic cell or a eukaryotic cell comprising the expression vector of claim 12, wherein said eukaryotic cell is a yeast cell, a fungi cell, a plant cell, or a mammalian cell.

14. A composition comprising the recombinant Ara h 6 variant polypeptide of claim 1 or the isolated nucleotide or modified nucleotide sequence of claim 10.

15. A method of inducing desensitization to peanuts and/or immunomodulation of a response to peanuts in a subject allergic to peanuts comprising administering to the subject the composition of claim 14.

16. (canceled)

17. (canceled)

18. A genetically modified peanut plant expressing the recombinant Ara h 6 variant polypeptide of claim 1.

19. The genetically modified peanut plant of claim 18, wherein said Ara h 6 variant is expressed from a heterologous nucleic acid.

20. The genetically modified peanut plant of claim 18, wherein expression of endogenous wild-type Ara h 6 allergen is reduced compared with a non-genetically modified peanut.

21. A processed food product comprising the Ara h 6 variant of claim 1.

22. The processed food product of claim 21, comprising a reduced amount of endogenous wild-type peanut Ara h 6 allergen.

23. The processed food product of claim 21, comprising a peanut harvested from the genetically modified plant of claim 18.
